(19) United States
(12) Patent Application Publication
(10) Pub. No.: US 2026/0098310 A1
(43) Pub. Date: Apr. 9, 2026

(54) METHODS AND DEVICES FOR ANALYZING SOIL MICROBIOTA

(71) Applicant: RhizeBio, Inc., Mount Holly, NC (US)

(72) Inventors: Douglas TOAL, Mount Holly, NC (US); Joshua TOAL, Belmont, NC (US)

(21) Appl. No.: 19/410,312

(22) Filed: Dec. 5, 2025

Related U.S. Application Data
(63) Continuation of application No. 19/348,874, filed on Oct. 3, 2025, now abandoned.
Application No. 19/348,874 is a continuation of application No. PCT/US2024/023862, filed on Apr. 10, 2024.
(60) Provisional application No. 63/458,279, filed on Apr. 10, 2023.
(60) Provisional application No. 63/588,498, filed on Oct. 6, 2023.

(51) Int. Cl. C12Q 1/689; C12Q 1/6869; G16B 25/10

(52) U.S. Cl. CPC C12Q 1/689; C12Q 1/6869; G16B 25/10; C12Q 2600/13

(57) ABSTRACT

Disclosed herein are systems, methods, and devices for collecting, processing, and analyzing a rhizobiome. Additionally, the systems, methods, and devices are used to generate recommendations based on a rhizobiome. The systems, methods, and devices disclosed herein can also be used to monitor a rhizobiome.

### CROSS-REFERENCE

This application is a continuation of U.S. application Ser. No. 19/348,874, filed on Oct. 3, 2025, entitled “METHODS AND DEVICES FOR ANALYZING SOIL MICROBIOTA,” which is a continuation of International Application No. PCT/US2024/023862, filed on Apr. 10, 2024, entitled “METHODS AND DEVICES FOR ANALYZING SOIL MICROBIOTA,” which claims the benefit of U.S. Provisional Application No. 63/458,279 filed Apr. 10, 2023, entitled “SOIL MICROBIOTA TRAP AND METHOD OF SOIL TESTING” and U.S. Provisional Application No. 63/588,498 filed Oct. 6, 2023, entitled “METHODS AND DEVICES FOR ANALYZING SOIL MICROBIOTA,” each of which is incorporated by reference herein in its entirety.

### BACKGROUND OF THE INVENTION

Soil microorganisms mediate many key ecosystem services that affect crop health, including nutrient cycling and pathogen suppression. Changes in the rhizobiome, microorganisms surrounding the root of a plant, are closely associated with plant developmental stages, genotypes, and can be predictive of pathogen presence. Biological inputs may stimulate or augment microbiological communities surrounding plant roots and promote crop health. Therefore, it is important to develop methods to characterize the rhizobiome and modulate the same using biological inputs.

### SUMMARY OF THE INVENTION

Disclosed herein are methods for characterizing one or more microorganisms in a sample. In some embodiments, the method comprises contacting an attractant to the sample, obtaining a rhizosphere of the sample and sequencing genetic material obtained from the rhizosphere 1-21 or 1-365 days after the contacting, thereby characterizing the one or more microorganisms of the sample. In some embodiments, contacting comprises planting, incubating, soaking, submerging, burying, suspending, or encapsulating the attractant within the sample. In some embodiments, the attractant comprises a seed, an ovule, a nut, a kernel, a pit, a pip, a bulb, a grain, or portions thereof. In some embodiments, the attractant comprises a microbe. In some embodiments, the microbe comprises a bacterium, an archaebacterium, an actinomycete, a fungus, an algal cell, a protozoan, a virus, a viroid, a nematode, or portions thereof. In some embodiments, the characterizing comprises aligning results of the sequencing. In some embodiments, the characterizing further comprises quantifying gene expression. In some embodiments, characterizing further comprises identifying one or more of the following: a species, genus, family, order, class, phylum, kingdom, or domain corresponding to the genetic material. In some embodiments, the characterizing further comprises use of machine learning. In some embodiments, obtaining the rhizosphere is carried out one or more times. In some embodiments, genetic material is obtained from the rhizosphere 1-5, 5-10, 10-15, 15-21, 22-30, 31-50, 51-100, 101-200, 201-300, or 301-365 days after the contacting. In some embodiments, characterizing identifies rhizosphere health, soil health from which the sample is obtained, plant health, relative abundance of genes important for plant health, or a farming characteristic in a location wherein from which the sample is obtained. In some embodiments, the sample comprises a microorganism. In some embodiments, the microorganism comprises a bacterium, an archaebacterium, an actinomycete, a fungus, an algal cell, a protozoan, a virus, a viroid, or a nematode. In some embodiments, the sample comprises a portion of a plant. In some embodiments, the plant comprises a root, stem, leaf, seed, or flower. In some embodiments, the sample comprises soil. In some embodiments, the soil comprises sand, clay, silt, chalk, peat, loam, or a combination thereof. In some embodiments, the sample comprises germination paper. In some embodiments, the sample comprises a liquid. In some embodiments, the rhizosphere comprises at least a portion of a plant root. In some embodiments, the root comprises a radicle, a taproot, a fibrous root, an adventitious root, an aerial root, a lateral root, or a modified root. In some embodiments, the genetic material comprises deoxyribonucleic acid (DNA) or ribonucleic acid (RNA). In some embodiments, the DNA comprises genomic DNA (gDNA), mitochondrial DNA (mtDNA), or circular DNA. In some embodiments, RNA comprises ribosomal RNA (rRNA), small nuclear RNA (snRNA), messenger RNA (mRNA), transfer RNA (tRNA), small nucleolar RNA (snoRNA), micro RNA (miRNA), or long noncoding RNA (lncRNA). In some embodiments, the genetic material is from a seed, a plant, a microorganism, a fungus, an insect, or an animal. In some embodiments, the sequencing comprises DNA sequencing, RNA sequencing, metagenomics sequencing, whole genome sequencing, whole exome sequencing, whole transcriptome sequencing, methylation sequencing, or chromatin studies. In some embodiments, the sequencing comprises next generation sequencing (NGS). In some embodiments, the sequencing comprises shotgun sequencing. In some embodiments, disclosed herein are devices for performing the method. In some embodiments, the device is cylindrical, permeable, and environmentally-resistant. In some embodiments, the device is reusable. In some embodiments, the device is disposable. In some embodiments, the device is a container for obtaining the sample. In some embodiments, disclosed herein is a system for performing the method. In some embodiments, the system comprises a controller, at least one processor, and an internet connection. In some embodiments, the system further comprises a sequencer. In some embodiments, the system comprises the device disclosed herein.

Disclosed herein are methods for generating and analyzing a sample of a rhizosphere. In some embodiments, the method comprises germinating a seed in conditions for the seed to release exudates, recruiting one or more microorganisms to the exudates to generate the rhizosphere, sequencing genetic material of the one or more microorganisms, thereby generating and analyzing a sample of a rhizosphere. In some embodiments, the seed comprises an open pollinated, non-hybrid, heirloom, hybrid, genetically modified organism (GMO) seed, or portions thereof. The seed may be obtained from a farmer. In some embodiments, the conditions comprise one or more of the following: soil composition, water availability, oxygen concentration, percent humidity, light, air composition, a greenhouse, a laboratory, a controlled setting, a field, a temperature below about 5° C., a temperature below about 10° C., a temperature below about 15° C., a temperature below about 20° C., or a temperature above about 20° C. In some embodiments, exudates comprise sugars, fatty acids, amino acids, small peptides, polypeptides, organic acids, growth factors, enzymes, nucleotides, hormones, vitamins, alcohols, phenolics, volatiles, or stimulants. In some embodiments, one or more microorganisms comprises a bacterium, an archaebacterium, an actinomycete, a fungus, an algal cell, a protozoan, a virus, a viroid, or a nematode. In some embodiments, sequencing is performed 1-21 or 1-365 days after germinating. In some embodiments, the sequencing is performed 1-5, 5-10, 10-15, 15-21, 22-30, 31-50, 51-100, 101-200, 201-300, or 301-365 days after germinating. In some embodiments, the sample comprises a microorganism. In some embodiments, the microorganism comprises a bacterium, an archaebacterium, an actinomycete, a fungus, an algal cell, a protozoan, a virus, a viroid, or a nematode. In some embodiments, the sample comprises a portion of a plant. In some embodiments, the plant comprises a root, stem, leaf, seed, or flower. In some embodiments, the sample comprises soil. In some embodiments, the soil comprises sand, clay, silt, chalk, peat, loam, or a combination thereof. In some embodiments, the sample comprises germination paper. In some embodiments, the sample comprises a liquid. In some embodiments, the rhizosphere comprises at least a portion of a plant root. In some embodiments, the root comprises a radicle, a taproot, a fibrous root, an adventitious root, an aerial root, a lateral root, or a modified root. In some embodiments, the genetic material comprises deoxyribonucleic acid (DNA) or ribonucleic acid (RNA). In some embodiments, the DNA comprises genomic DNA (gDNA), mitochondrial DNA (mtDNA), or circular DNA. In some embodiments, RNA comprises ribosomal RNA (rRNA), small nuclear RNA (snRNA), messenger RNA (mRNA), transfer RNA (tRNA), small nucleolar RNA (snoRNA), micro RNA (miRNA), or long noncoding RNA (lncRNA). In some embodiments, the genetic material is from a seed, a plant, a microorganism, a fungus, an insect, or an animal. In some embodiments, the sequencing comprises DNA sequencing, RNA sequencing, metagenomics sequencing, whole genome sequencing, whole exome sequencing, whole transcriptome sequencing, methylation sequencing, or chromatin studies. DNA quality may be evaluated using a Qubit 4 Fluorometer 1X High Sensitivity dsDNA assay. Acceptable concentrations of DNA for downstream analysis may be 10 ng/uL or more. DNA samples may be diluted with molecular biology-grade water in order to continue analysis. In some embodiments, the sequencing comprises next generation sequencing (NGS). In some embodiments, the sequencing comprises shotgun sequencing. In some embodiments, disclosed herein are devices for performing the method. In some embodiments, the device is cylindrical, permeable, and environmentally-resistant. In some embodiments, the device is reusable. In some embodiments, the device is disposable. In some embodiments, the device is a container for obtaining the sample. In some embodiments, disclosed herein is a system for performing the method. In some embodiments, the system comprises a controller, at least one processor, and an internet connection. In some embodiments, the system further comprises a sequencer. In some embodiments, the system comprises the device disclosed herein.

Disclosed herein are methods for analyzing a rhizosphere. In some embodiments, the method comprises obtaining a sample from the rhizosphere; sequencing genetic material obtained from the sample; performing bioinformatic analysis on results of the sequencing; and identifying beneficial microorganisms in said rhizosphere, wherein the beneficial microorganisms confer benefit to a plant. In some embodiments, beneficial microorganisms comprise one of more of the genera Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, or Streptomyces. In some embodiments, the plant comprises a crop plant, a non-crop plant, a cultivated plant, or a non-cultivated plant. In some embodiments, a benefit comprises, resistance to an environmental stressor, resistance to a disease, pest, or pesticide, reduced susceptibility to a disease, pest, or pesticide; or improved nutrient uptake. In some embodiments, environmental stressor comprises drought, flood, salinity, heat, cold, ozone, UV radiation, heavy metals, or nutrient deficiency. In some embodiments, the disease comprises stress, root rot, damping-off, vascular wilt, nutritional deficiency, salt injury, or infection caused by fungi, oomycetes, bacteria, viruses, viroids, virus-like organisms, phytoplasmas, protozoa, nematodes, or parasitic plants. In some embodiments, the pest comprises an aphid, a thrip, a mite, a leaf miner, a fly, an earwig, a gnat, a mealybug, a worm, a beetle, a caterpillar, a cicada, a slug, a snail, a moth, a cricket, an ant, or a nematode. In some embodiments, the pesticide comprises one or more of an organochlorine, an organophosphate, an organosulfur, a carbamate, a formamidine, a dinitrophenol, an organotin, a pyrethroid, a nicotinoid, a spinosyn, a pyrazole, a pyridazinone, a quinazoline, a botanical, a synergist, an antibiotic, a fumigant, an inorganic, a biorational, a benzoylurea, an herbicide, an insecticide, or a fungicide. In some embodiments, the nutrient comprises nitrogen, phosphorus, potassium, calcium, magnesium, sulfur, iron, zinc, manganese, copper, boron, molybdenum, or chlorine. In some embodiments, the sample is obtained one or more times. In some embodiments, the sample is obtained over multiple days. In some embodiments, the sample is obtained 1-21 or 1-365 days after germination. In some embodiments, the sample is obtained 1-5, 5-10, 10-15, 15-21, 22-30, 31-50, 51-100, 101-200, 201-300, or 301-365 days after germination. In some embodiments, the method further comprise implementing the beneficial plant management technique on the plant. In some embodiments, following the implementing, the plant exhibits an improved phenotype of agronomic interest comprising disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increased yield, increased yield under water-limited conditions, health enhancement, vigor improvement, growth improvement, photosynthetic capability improvement, nutrition enhancement, altered protein content, altered oil content, increased biomass, increased shoot length, increased root length, improved root architecture, increased seed weight, altered seed carbohydrate composition, altered seed oil composition, number of pods, delayed senescence, stay-green, or altered seed protein composition. In some embodiments, the bioinformatic analysis comprises sequence alignment. In some embodiments, the bioinformatic analysis comprises the use of machine learning. In some embodiments, the bioinformatic analysis comprises identifying based on an organism in said sample: a gene function, wherein said gene function is associated with nitrogen cycling, phosphorous cycling, carbon cycling, drought resistance, polymer degradation, or oxygen availability; a metabolic pathway, wherein said metabolic pathway comprises secreted proteases or urea mineralization and is determined by measuring a constituent gene; or an agronomic attribute, wherein said agronomic attribute comprises nitrogen mineralization. In some embodiments, the sample comprises a microorganism. In some embodiments, the microorganism comprises a bacterium, an archaebacterium, an actinomycete, a fungus, an algal cell, a protozoan, a virus, a viroid, or a nematode. In some embodiments, the sample comprises a portion of a plant. In some embodiments, the plant comprises a root, stem, leaf, seed, or flower. In some embodiments, the sample comprises soil. In some embodiments, the soil comprises sand, clay, silt, chalk, peat, loam, or a combination thereof. In some embodiments, the sample comprises germination paper. In some embodiments, the sample comprises a liquid. In some embodiments, the rhizosphere comprises at least a portion of a plant root. In some embodiments, the root comprises a radicle, a taproot, a fibrous root, an adventitious root, an aerial root, a lateral root, or a modified root. In some embodiments, the genetic material comprises deoxyribonucleic acid (DNA) or ribonucleic acid (RNA). In some embodiments, the DNA comprises genomic DNA (gDNA), mitochondrial DNA (mtDNA), or circular DNA. In some embodiments, RNA comprises ribosomal RNA (rRNA), small nuclear RNA (snRNA), messenger RNA (mRNA), transfer RNA (tRNA), small nucleolar RNA (snoRNA), micro RNA (miRNA), or long noncoding RNA (lncRNA). In some embodiments, the genetic material is from a seed, a plant, a microorganism, a fungus, an insect, or an animal. In some embodiments, the sequencing comprises DNA sequencing, RNA sequencing, metagenomics sequencing, whole genome sequencing, whole exome sequencing, whole transcriptome sequencing, methylation sequencing, or chromatin studies. In some embodiments, the sequencing comprises next generation sequencing (NGS). In some embodiments, the sequencing comprises shotgun sequencing. In some embodiments, disclosed herein are devices for performing the method. In some embodiments, the device is cylindrical, permeable, and environmentally-resistant. In some embodiments, the device is reusable. In some embodiments, the device is disposable. In some embodiments, the device is a container for obtaining the sample. In some embodiments, disclosed herein is a system for performing the method. In some embodiments, the system comprises a controller, at least one processor, and an internet connection. In some embodiments, the system further comprises a sequencer. In some embodiments, the system comprises the device disclosed herein.

Disclosed herein are methods for bioinformatically analyzing a sample of a rhizosphere. In some embodiments, the method comprises obtaining the sample from within 100 millimeters of a root of a plant, sequencing genetic material obtained from the sample; and performing bioinformatic analysis on results of said sequencing. In some embodiments, the sample is obtained from within less than about 5, 10, 15, 20, 25, 30, 40, 50, 60, 70, 80, 90, or 100 millimeters of a root of a plant. In some embodiments, a sample is obtained from less than about 0.1, 0.2, 0.3, 0.4, 0.5, 0.6, 0.7, 0.8, 0.9, 1.0, 1.5, 2.0, 2.5, or 3.0 millimeters of the root of the plant. In some embodiments, the sample comprises a portion of said root. In some embodiments, the root comprises a radicle, a taproot, a fibrous root, an adventitious root, an aerial root, a lateral root, or a modified root. In some embodiments, the plant comprises a crop plant, a non-crop plant, a cultivated plant, or a non-cultivated plant. Crop plants may include but are not limited to peanut, chickpea, potato, almond, sweet potato, grape, hemp, corn, cotton, tomato, carrot, cherry, soybean, onion, barley, sunflower, date, canola, pea, blueberry, peach, hazelnut, brassica, wheat, strawberry, sorghum, bean, citrus, cucumber, pear, artichoke, pepper, alfalfa, lettuce, melon, plum, walnut, apple, sugarcane, sugarbeet, beet, cereal, rye cereal, lentil, watermelon, pecan, hops, flax, turf, oat, pistachio, avocado, rice, celery, blackberry, garlic, squash, spinach, raspberry, asparagus, olive, and cilantro. In some embodiments, the sample is obtained one or more times. In some embodiments, the sample is obtained over multiple days. In some embodiments, the sample is obtained 1-21 or 1-365 days after germination. In some embodiments, the sample is obtained 1-5, 5-10, 10-15, 15-21, 22-30, 31-50, 51-100, 101-200, 201-300, or 301-365 days after germination. In some embodiments, the method further comprises implementing the beneficial plant management technique on the plant. In some embodiments, following the implementing, the plant exhibits an improved phenotype of agronomic interest comprising disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increased yield, increased yield under water-limited conditions, health enhancement, vigor improvement, growth improvement, photosynthetic capability improvement, nutrition enhancement, altered protein content, altered oil content, increased biomass, increased shoot length, increased root length, improved root architecture, increased seed weight, altered seed carbohydrate composition, altered seed oil composition, number of pods, delayed senescence, stay-green, or altered seed protein composition. In some embodiments, the bioinformatic analysis comprises sequence alignment. In some embodiments, the bioinformatic analysis comprises the use of machine learning. In some embodiments, the bioinformatic analysis comprises identifying based on an organism in said sample: a gene function, wherein said gene function is associated with one or more of nitrogen cycling, phosphorous cycling, carbon cycling, drought resistance, polymer degradation, or oxygen availability; a metabolic pathway, wherein said metabolic pathway comprises secreted proteases or urea mineralization and is determined by measuring a constituent gene; or an agronomic attribute, wherein said agronomic attribute comprises nitrogen mineralization. In some embodiments, the sample comprises a microorganism. In some embodiments, the microorganism comprises a bacterium, an archaebacterium, an actinomycete, a fungus, an algal cell, a protozoan, a virus, a viroid, or a nematode. In some embodiments, the sample comprises a portion of a plant. In some embodiments, the plant comprises a root, stem, leaf, seed, or flower. In some embodiments, the sample comprises soil. In some embodiments, the soil comprises sand, clay, silt, chalk, peat, loam, or a combination thereof. In some embodiments, the sample comprises germination paper. In some embodiments, the sample comprises a liquid. In some embodiments, the rhizosphere comprises at least a portion of a plant root. In some embodiments, the root comprises a radicle, a taproot, a fibrous root, an adventitious root, an aerial root, a lateral root, or a modified root. In some embodiments, the genetic material comprises deoxyribonucleic acid (DNA) or ribonucleic acid (RNA). In some embodiments, the DNA comprises genomic DNA (gDNA), mitochondrial DNA (mtDNA), or circular DNA. In some embodiments, RNA comprises ribosomal RNA (rRNA), small nuclear RNA (snRNA), messenger RNA (mRNA), transfer RNA (tRNA), small nucleolar RNA (snoRNA), micro RNA (miRNA), or long noncoding RNA (lncRNA). In some embodiments, the genetic material is from a seed, a plant, a microorganism, a fungus, an insect, or an animal. In some embodiments, the sequencing comprises DNA sequencing, RNA sequencing, metagenomics sequencing, whole genome sequencing, whole exome sequencing, whole transcriptome sequencing, methylation sequencing, or chromatin studies. In some embodiments, the sequencing comprises next generation sequencing (NGS). In some embodiments, the sequencing comprises shotgun sequencing. In some embodiments, disclosed herein are devices for performing the method. In some embodiments, the device is cylindrical, permeable, and environmentally-resistant. In some embodiments, the device is reusable. In some embodiments, the device is disposable. In some embodiments, the device is a container for obtaining the sample. In some embodiments, disclosed herein is a system for performing the method. In some embodiments, the system comprises a controller, at least one processor, and an internet connection. In some embodiments, the system further comprises a sequencer. In some embodiments, the system comprises the device disclosed herein.

In some embodiments, disclosed herein are methods for improving a plant characteristic. In some embodiments, the method comprises, obtaining a sample from a rhizosphere of the plant, sequencing genetic material obtained from the sample, performing bioinformatic analysis on results of the sequencing; and determining a beneficial plant management technique for the plant to improve a plant characteristic based at least on said bioinformatic analysis, wherein the plant characteristic comprises plant yield or plant health. The beneficial plant management technique may comprise identifying a beneficial growth temperature, humidity range, soil composition, soil treatment composition, nutrient composition, plant food, biofertilizer or a biostimulant. In some embodiments, the soil treatment composition comprises one or more of Trichoderma harzianum, Bacillus amyloliquefaciens, Bacillus subtilis, Myxococcus xanthus, Wickerhamomyces anomalus, Azotobacter vinelandii, Frateuria aurantia, Pseudomonas chlororaphis, Starmerella bombicola, Saccharomyces boulardii, Pichia occidentalis, Pichia kudriavzevii, or Meyerozyma guilliermondii. In some embodiments, the biostimulant provides a benefit to a plant. In some embodiments, the benefit comprises one or more of increased growth, increased disease resistance, increased nutrition efficiency, increased stress tolerance, or improved crop quality. In some embodiments, the biofertilizer comprises microbes, wherein the microbes comprise one or more of the following genera: Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, or Streptomyces. The beneficial plant management technique may comprise a foliar biological. A foliar biological may comprise a fertilizer or biostimulant which is sprayed directly onto a plant.

In some embodiments, the methods disclosed herein may further comprise generating a score based on analysis of a sample. Said score may take into account one or more factors selected from observed genera, community evenness, mycorrhizae, fungal/bacteria ratio (F/B ratio), rare biosphere, nitrification, denitrification, nitrogen fixation, phosphorous cycling, polymer degradation, drought resistance, disturbance, total score, and any combination thereof. In some embodiments, analysis of genes disclosed in Tables 1-32 may be used to generate said score.

In some embodiments, the plant comprises a crop plant, a non-crop plant, a cultivated plant, or a non-cultivated plant. In some embodiments, the sample is obtained one or more times. In some embodiments, the sample is obtained over multiple days. In some embodiments, the sample is obtained 1-21 or 1-365 days after germination. In some embodiments, the sample is obtained 1-5, 5-10, 10-15, 15-21, 22-30, 31-50, 51-100, 101-200, 201-300, or 301-365 days after germination. In some embodiments, the method further comprise implementing the beneficial plant management technique on the plant. In some embodiments, following the implementing, the plant exhibits an improved phenotype of agronomic interest comprising disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increased yield, increased yield under water-limited conditions, health enhancement, vigor improvement, growth improvement, photosynthetic capability improvement, nutrition enhancement, altered protein content, altered oil content, increased biomass, increased shoot length, increased root length, improved root architecture, increased seed weight, altered seed carbohydrate composition, altered seed oil composition, number of pods, delayed senescence, stay-green, or altered seed protein composition. In some embodiments, the bioinformatic analysis comprises sequence alignment. In some embodiments, the bioinformatic analysis comprises the use of machine learning. In some embodiments, the bioinformatic analysis comprises identifying based on an organism in said sample: a gene function, wherein said gene function is associated with nitrogen cycling, phosphorous cycling, carbon cycling, drought resistance, polymer degradation, or oxygen availability; a metabolic pathway, wherein said metabolic pathway comprises secreted proteases or urea mineralization and is determined by measuring a constituent gene; or an agronomic attribute, wherein said agronomic attribute comprises nitrogen mineralization. The sample may comprise a microorganism. The microorganism may comprise a bacterium, an archaebacterium, an actinomycete, a fungus, an algal cell, a protozoan, a virus, a viroid, or a nematode. The sample may comprise a portion of a plant. In some embodiments, the plant comprises a root, stem, leaf, seed, or flower. The sample may comprise soil. In some embodiments, the soil comprises sand, clay, silt, chalk, sandy loam, clay loam, peat, loam, or a combination thereof. In some embodiments, the sample comprises germination paper. In some embodiments, the sample comprises a liquid. In some embodiments, the rhizosphere comprises at least a portion of a plant root. In some embodiments, the root comprises a radicle, a taproot, a fibrous root, an adventitious root, an aerial root, a lateral root, or a modified root. In some embodiments, the genetic material comprises deoxyribonucleic acid (DNA) or ribonucleic acid (RNA). In some embodiments, the DNA comprises genomic DNA (gDNA), mitochondrial DNA (mtDNA), or circular DNA. In some embodiments, RNA comprises ribosomal RNA (rRNA), small nuclear RNA (snRNA), messenger RNA (mRNA), transfer RNA (tRNA), small nucleolar RNA (snoRNA), micro RNA (miRNA), or long noncoding RNA (lncRNA). In some embodiments, the genetic material is from a seed, a plant, a microorganism, a fungus, an insect, or an animal. In some embodiments, the sequencing comprises DNA sequencing, RNA sequencing, metagenomics sequencing, whole genome sequencing, whole exome sequencing, whole transcriptome sequencing, methylation sequencing, or chromatin studies. In some embodiments, the sequencing comprises next generation sequencing (NGS). In some embodiments, the sequencing comprises shotgun sequencing. In some embodiments, disclosed herein are devices for performing the method. In some embodiments, the device is cylindrical, permeable, and environmentally-resistant. In some embodiments, the device is reusable. In some embodiments, the device is disposable. In some embodiments, the device is a container for obtaining the sample. In some embodiments, disclosed herein is a system for performing the method. In some embodiments, the system comprises a controller, at least one processor, and an internet connection. In some embodiments, the system further comprises a sequencer. In some embodiments, the system comprises the device disclosed herein.

Further disclosed herein are kits comprising materials for performing the methods disclosed herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1D depicts a non-limiting example of a device for collecting a soil sample for analysis.

FIG. 2 depicts a non-limiting workflow for analysis of sequence data.

FIG. 3 depicts a non-limiting workflow for organization and characterization of the output generated in FIG. 2.

FIG. 4 depicts an exemplary computer system for use.

FIG. 5 depicts an application provision system.

FIG. 6 depicts an application provision system.

FIG. 7 depicts a non-limiting exemplary report generated from analysis of samples.

FIG. 8 depicts a graph of taxonomic dissimilarity using Kraken2.

FIG. 9A-FIG. 9B depict graphs of temperature enriched genera.

FIG. 10A-FIG. 10B depict graphs of site enriched genera.

FIG. 11 depicts a graph of gene function dissimilarity using KEGG.

FIG. 12A-FIG. 12B depict temperature enriched KEGG orthologs.

FIG. 13 depicts gene function dissimilarity using COG.

FIG. 14A-FIG. 14B depict graphs of serine/threonine protein kinase quantification.

FIG. 15A-FIG. 15B depict graphs of tricarboxylic transport membrane protein quantification.

FIG. 16A-FIG. 16C depict quantification of alpha,alpha-trehalase (FIG. 16A), multiple sugar transport system (FIG. 16B), and Streptomyces (FIG. 16C).

FIG. 17A-FIG. 17B depict graphs of quantification of nitrification and denitrification.

FIG. 18A-FIG. 18B depict graphs of quantification of nitrifiers.

FIG. 19 depicts a graph of effect of several treatments on microbiome characteristics.

FIG. 20 depicts a graph of effect of a treatment on microbiome characteristics.

FIG. 21 depicts a graph of a comparison of Rhize score with crop yield.

### DETAILED DESCRIPTION OF THE INVENTION

The soil microbiome includes thousands of organisms, including bacteria, fungi, and nematodes, among other microorganisms. These organisms uniquely impacts the soil and the plants grown therein. Identifying the microorganisms in soil or around the root of the plant can provide insight into plant health, soil health, and how certain treatment can impact the same. Metagenomics (also referred to as environmental genomics or community genomics) is the science of describing the profile of microbiomes detected in a biological sample such as soil, including the functional genes underlying microbial metabolism. In some embodiments, metagenomic analysis is used to identify and characterize the microbiome in or a sample.

Although genetic analysis of bulk field soil is available to provide details on a soil microbiome, this analysis fails to consider the more important biological role played by the soil microorganisms within the root rhizosphere, or rhizomicrobiome. Moreover, the analysis of bulk soil leads to challenges in distinguishing between active microbes, dormant microbes, and relic DNA in soil. By measuring the microbial composition and genetic potential of the rhizomicrobiome before and after implementation of a treatment to the soil and/or plants, it is possible to assess plant health, soil health and the effect of the inputs on the same.

Metagenomic analysis of the microbiome within rhizosphere samples may reveal functionally relevant genes as opposed to bulk soil sampling, which may reveal mostly irrelevant genes. In this regard, it is important to develop sample collection and DNA extraction strategies that target plant-microbiome interactions in the rhizosphere.

Disclosed herein are methods, devices, and systems for sampling and analyzing the rhizomicrobiome.

### Sample Collection

Disclosed herein are methods for collection of one or more samples. In some embodiments, the sample is a composite sample. The sample may be collected from 1, 2, 3, 4, 5, 6, 7, 8, 9, 10, 11, 12, 13, 14, 15, 16, 17, 18, 19, 20, or more locations. The locations may be indoors, outdoors, in a greenhouse, in a laboratory, in a controlled setting, in a field, in a planter, in a garden, or any combination thereof. The sample may be collected from the surface of an area of soil. The sample may be collected from 1 inch, 2 inches, 3 inches, 4 inches, 5 inches, 6 inches, 7 inches, 8 inches, 9 inches, 10 inches, 11 inches, 12 inches or more, or any distance in between, beneath the soil surface. Samples may be collected from 13, 14, 15, 16, 17, 18, 19, 20, 21, 22, 23, 24, or more inches below the soil surface. One or more samples may be combined to create a composite sample. The composite sample may comprise samples obtained from 15 locations in an area of soil. The composite sample may be mixed and homogenized. Approximately 1 cup, 2 cups, 3 cups, 4 cups, 5 cups, 6 cups, 7 cups, 8 cups, 9 cups, 10 cups of soil or more, or any amount in between, may be collected for a composite sample. Approximately 0.1 gallon, 0.2 gallons, 0.3 gallons, 0.4 gallons, 0.5 gallons, 0.6 gallons, 0.7 gallons, 0.8 gallons, 0.9 gallons, 1 gallon of soil or more, or any amount in between, may be collected for a composite sample. Metadata may be collected along with a soil sample. Metadata may comprise soil type, irrigation methods, crop identity, location, latitude, longitude, collection date, tillage, management techniques, sample collection depth, intended use, and/or type of farm. Type of soil may comprise clay, silt, sand, loam, sandy loam, clay loam, peat, or any combination thereof. Tillage may comprise the use of a moldboard plow, chisel plow, straight point, disk, or any other tool which may possibly be used for tillage. Management techniques may be biodynamic, conservation-oriented, regenerative, soil health-oriented, organic, or other. Plant management techniques may be determined based on calculation of a total score as described herein.

In some embodiments, samples are refrigerated for storage. Samples may be stored long-term at approximately −20° C. (−4° F.). Samples may be stored in a refrigerator or refrigerated space for up to 1, 2, 3, 4, 5, 6, or 7 days at from 0° F. to 6° F. (32-48° C.). Samples may be held for up to 1, 2, 3, 4 or 5 days at room temperature. Samples may be shipped with ice packs or dry ice. Samples may be stored in DNA/RNA Shield nucleic acid neutralization buffer.

In some embodiments, the tools used to collect or handle samples are disinfected in between uses. Standard sterile techniques may be used to disinfect tools or equipment disclosed herein. Surfaces, tools, and equipment disclosed herein may be cleaned using 70% isopropanol. Sterile techniques may comprise 70% EtOH, 10% bleach, water, heat, UV radiation, autoclaving, baking, filtration, or any combination thereof. Disinfection or sterilizing of tools and equipment disclosed herein may be used to avoid cross-contamination. Disposable gloves may be used for sample collection. A core sampler may be used to collected samples. A spade or shovel may be used to collected samples. A gloved hand, clean scoopula, or pouring from a bag may be used to transfer soil.

In some embodiments, the rhizosphere may be collected. Rhizosphere collection may comprise uprooting of a plant or a crop. An entire plant or crop may be uprooted. From 1 to 8 crops may be uprooted per zone. A zone may comprise a homogenous or similarly performing region of soil. A crop may be uprooted including its entire root system. Loose soil may be removed from the root system. The entire root system along with the crop may be placed in a bag for analysis. In some embodiments, the root mass and stalk are not needed. In some embodiments, only the root mass and leaves are bagged. The root mass and leaves may be bagged separately.

Samples may be collected early in the growing season. Samples may be collected 1 week after foliar application of a biological. Treated and untreated plants may be compared for analysis. Treated plants may be obtained from the same zone or replicated strip. Untreated plants may be obtained from the same zone or replicated strip. Treated and untreated plants may be obtained from the same zone or replicated strip.

### Seed Growing

Disclosed herein are techniques for growing seeds. In some embodiments, seeds are grown in a greenhouse. Environmental factors may be controlled in a greenhouse. Environmental factors may comprise temperature, humidity, and light. The greenhouse may comprise a humidifier. The humidifier may be filled with distilled water.

Seeds may receive a variable amount of water. Seeds may receive a constant amount of water. Initial watering of a seed may comprise from 1, 2, 3, 4, 5, 6, 7, 8, 9, or 10 mL of water, or to soil saturation. Water may comprise molecular grade or distilled water. Water may be applied to a seed or soil using a serological pipette.

### Rhizosphere

Disclosed herein are methods, devices, and systems for sampling and analyzing microorganisms present in the rhizosphere. The rhizosphere is the zone of soil wherein a plant's root system can grow and can absorb water and nutrients. There are diverse communities of microorganisms comprising a myriad of species within the rhizosphere that can coexist with each other and with plant roots. This coexistence can form a complex network of relationships, commonly referred to as the rhizomicrobiome or rhizobiome. This community of microorganisms, or microbiome, performs key ecosystem functions. These functions can aid plant growth and health. Non-limiting examples of these functions include but are not limited to fixing and cycling of nutrients, immune modulation, pest and disease control, and water retention. The rhizosphere may play an important role in plant nutrition and pathogenesis. In some embodiments, the rhizosphere can control the persistence, mobility, and bioavailability of nutrients as well as contaminants in soils. In some embodiments, communities of microorganisms also exist in and on above-ground plant parts, in water sources, and in the air. Composition of the community may be influenced by factors including but not limited to soil characteristics, soil type, humidity, water content, pH, climate, temperature, organic carbon availability, oxygen availability, and concentrations of chemicals in soil. Nutrients may comprise but are not limited to one or more of nitrogen, oxygen, phosphorous, calcium, iron, sodium, potassium, sulfur, magnesium, chlorine, hydrogen, iron, boron, manganese, zinc, copper, nickel, and molybdenum. Cycling of nutrients may comprise but is not limited to one or more of the following processes: sorption, desorption, precipitation, dissolution, plant uptake, tile flow, sedimentation, resuspension, erosion, runoff, leaching, mineralization, immobilization, fixation, assimilation, evaporation, respiration, recycling, lysis, transpiration, combustion, and deposition.

In some embodiments, the rhizosphere can refer to the environment adjacent to the roots of one or more plants. The rhizosphere may comprise soil. The rhizosphere may comprise exudates, secretions, or both. In some embodiments, these exudates, secretions, or both can be released from a seed during the process of germination. In some embodiments, these exudates, secretions, or both can be released by microorganisms inhabiting the rhizosphere. In some embodiments, the rhizosphere may extend approximately 100 millimeters away from the surface of a given plant root. In some embodiments, the rhizosphere may refer to the area less than about 100 millimeters, less than about 90 millimeters, less than about 80 millimeters, less than about 70 millimeters, less than about 60 millimeters, less than about 50 millimeters, less than about 40 millimeters, less than about 30 millimeters, less than about 25 millimeters, less than about 20 millimeters, less than about 15 millimeters, less than about 10 millimeters, less than about 5 millimeters, less than about 3 millimeters, less than about 1 millimeter, less than about 0.9 millimeters, less than about 0.8 millimeters, less than about 0.7 millimeters, less than about 0.6 millimeters, less than about 0.5 millimeters, less than about 0.4 millimeters, less than about 0.3 millimeters, less than about 0.2 millimeters, or less than about 0.1 millimeters from a surface of a plant root. The rhizosphere may comprise up to 0.5 cups of soil remaining on a plant's roots after removal from soil.

In some embodiments, a plant or seed may be grown in or on an environment other than soil. The environment of a seed or plant may comprise but is not limited to one or more of soil, water, air, germination paper, light, humidity, microorganisms, fertilizer, nutrients, or any combination thereof. The soil may comprise one or more of sand, clay, silt, chalk, peat, sandy loam, clay loam, loam, or any combination thereof.

In some embodiments, a plant or seed may be grown inside of a device. Roots growing outside of the device may be removed. Plants harvested from the device may be cut at the root-shoot interface. A cotton-tipped swab may be used to gently brush roots and remove rhizospheric soil. Rhizospheric soil may surround and/or adhere to a root. Phosphate buffered saline (PBS) may be used to remove soil from a root. Roots may be sonicated to remove the rhizosphere. Roots may be washed one, two, or three or more times. DNA extraction from the rhizosphere may be done using the Qiagen PowerSoil Pro protocol or a variation thereof.

In some embodiments, the plant may be grown inside the device in a field or in a pot. The plant may be removed from the device and placed on a clean, flat surface. The roots that have grown outside of the device or outside of the pot may be cut. The plant may be cut at the root-shoot interface. A cotton-tipped swab may be used to gently brush the roots, leaving 1 mm of rhizospheric soil immediately surrounding the root. The roots with the rhizospheric soil may be placed in a 50 mL conical tube with about 25 mL of molecular-grade water and shaken gently to remove the soil from the roots and create a rhizospheric soil slurry. Roots may be retained and stored at about 4° C. about 4 mL aliquots of the rhizospheric soil slurry each may be added to 2 mL centrifuge about tubes. The aliquots may be maintained and stored at about 4° C. The aliquots may be centrifuged at 10,000 g for about 30 seconds to form a soil pellet. The clear supernatant may be discarded, leaving only the soil pellet. The pellet may be stored at about 4° C. if it is not immediately used for DNA extraction. DNA extraction may be performed on the soil pellet using the Qiagen PowerSoil Pro Protocol or a variation thereof. DNA can be quantified using a Qubit 4 Fluorometer 1× High Sensitivity dsDNA assay. Acceptable concentrations of rhizospheric DNA for downstream sequencing can be 10 ng/uL or more. If the DNA is too concentrated, it may be diluted in order to proceed with analysis.

In some embodiments, soil may be collected from a field, a garden, a greenhouse, a laboratory, a controlled setting, a pot, a planter, or a vessel for growing plants. The soil may be analyzed. A volumetric quart of desired bulk soil may be collected into a clean plastic bag. The soil may be sieved using a sieve with aperture openings appropriate for the desired crop type. In some cases, the aperture openings may be 5 mm. Subsamples of the soil may be aliquot into 2 mL microcentrifuge tubes. Each tube may contain approximately 825 milligrams of soil. Aliquots may be stored at about −20° C. The remaining soil may be stored at about 4° C. Aliquots may be thawed at room temperature before proceeding. 1.5 mL of PBS may be added to each aliquot. The tubes containing the soil and the PBS may be placed on a Hula mixer for 1 hour under the following conditions: 360° rotation for one hour, with about a 10 second shake step every 45 seconds. The tubes may then be centrifuged at about 600 g for about 4 minutes to separate larger soil particles from the PBS-microbiome suspension. The supernatant slurry may be transferred to fresh 2 mL microcentrifuge tubes. Seeds may be inoculated with the soil slurry on germination paper. Seeds on germination paper may be inoculated with either the inoculant containing the soil slurry or control PBS buffer. Seeds may be pressed between layers of germination paper and maintained in a greenhouse for germination. Germination may occur over from 1 to 10 days. Seedlings may be removed from the greenhouse. Seeds and seed casings may be removed from the plant tissue. Plant tissue may be retained and transferred to a piece of parchment paper and placed inside of a drying bag to dry. Drying may occur over about 48 hours at about 45° C. Plant tissue may be stored at about −20° C. until ready for DNA extraction. Seedling tissue may be thawed for DNA extraction. DNA extraction may be performed using a Qiagen PowerSoil Pro Protocol or a variation thereof. DNA can be quantified using a Qubit 4 Fluorometer 1× High Sensitivity dsDNA assay. Acceptable concentrations of rhizospheric DNA for downstream sequencing can be 10 ng/uL or more. If the DNA is too concentrated, it may be diluted in order to proceed with analysis.

In some embodiments, bulk soil may be subjected to DNA collection and DNA sequencing for comparison with rhizobiome soil. DNA collected from bulk soil may be extracted using a Qiagen PowerSoil Pro Protocol or a variation thereof.

In some embodiments, a plant or seed may be grown in a pot. Size of the pot may be selected based on the amount of soil needed as input for analysis. Pots may be autoclaved before use. Pots may be filled with soil. A gloved finger or a cotton swab may be used to create a divot in the soil for the seed. The divot may be about ¼ to about ½ inch deep, or more. One seed may be grown per pot. Seeds may be selected to be representative of a batch of seeds. Seeds used in a given trial may be the same weight, size, and/or color. Seeds may be covered with soil.

In some embodiments, on-seed treatment may be applied. On-seed treatment may comprise biological treatment. On-seed treatment may comprise fertilizer. On-seed treatment may be in liquid or powder form. One or more seeds may be placed in a bag containing a biological treatment and shaken. Seeds or seed coats may be coated with the biological treatment. The on-seed or biological treatment may be applied to both the seed and the soil. Biological treatment may be diluted before being applied to a seed or soil. The biological or on-seed treatment may comprise algae. For purposes of the present disclosure, the terms biological input, biological treatment, and on-seed treatment may be interchangeable. Biological or on-seed treatment may comprise fulvic, molasses, humic, yucca, kelp, compost, nitrogen, phosphorous, potassium, a combination of nitrogen and phosphorous and potassium (NPK), fertilizer, or any combination thereof.

In some embodiments, change in the core rhizobiome of a given plant can be closely associated with plant developmental stages and/or genotypes. In some embodiments, the core rhizobiome can be predictive of pathogen presence. In some embodiments, early soil rhizosphere formation can reveal clues to plant health and disease susceptibility. In some embodiments, the initial plant rhizobiome composition and/or functioning can predetermine future plant health. In some embodiments, the rhizosphere can be sampled before contacting a plant environment with a plant or seed. In some embodiments, the rhizosphere can be sampled within 1 day, within 2 days, within 3 days, within 4 days, within 5 days, within 6 days, within 7 days, within 8 days, within 9 days, within 10 days, within 11 days, within 12 days, within 13 days, within 14 days, within 15 days, within 16 days, within 17 days, within 18, within 19 days, within 20 days, within 21 days, within 30 days, within 50 days, within 100 days, within 200 days, within 300 days, or within 365 days of the contacting of a plant or seed environment with a plant or seed, respectively. In some embodiments, a rhizosphere is sampled at multiple points of a plant/seed development. In some embodiments, a rhizosphere sample is analyzed after collection of the sample by a method disclosed herein. In some embodiments, analysis of a rhizosphere sample can identify microorganisms of the rhizosphere, determine soil health, determine plant health, or inform how to treat plant/soil to improve plant/soil health.

In some embodiments, microorganisms of the rhizosphere perform many functions. These functions include but are not limited to improve nitrogen fixation, release phosphate from the soil organic matter, release phosphate from the inorganic forms of phosphate (e.g. rock phosphate), “fix carbon” in the root microsphere, live in the rhizosphere of the plant, increase the number of nodules on the plant roots, elicit plant defensive responses such as ISR (induced systemic resistance), elicit plant defensive responses such as SAR (systemic acquired resistance), compete with microorganisms deleterious to plant growth or health, change the color of one or more part of the plant, or change the chemical profile of the plant, its smell, taste or one or more other quality.

In some embodiments, the rhizosphere of a plant can assist the plant in absorbing nutrients from the surrounding soil. The rhizosphere of the plant can provide nutrients from the surrounding soil more readily to the plant. The rhizosphere of the plant can increase the number of nodules on the plant roots thereby increasing the number of symbiotic nitrogen fixing bacteria (e.g. Rhizobium species) per plant. The rhizosphere of the plant can increase the number of nodules on the plant roots thereby increasing the amount of nitrogen fixed by the plant. ISR can help the plant resist the invasion and spread of pathogenic microorganisms. SAR can help the plant resist the invasion and spread of pathogenic microorganisms. The rhizosphere of the plant can compete with deleterious microorganisms by antagonism. The rhizosphere of the plant can compete with deleterious microorganism's competitive utilization of resources such as nutrients and/or space.

In some embodiments, microorganisms of the rhizosphere can comprise one or more of a bacterium, an archaebacterium, an actinomycete, a fungus, an algal cell, a protozoan, a virus, a viroid or a nematode. Microorganisms of the rhizosphere may comprise but are not limited to one or more of the genera Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, or Streptomyces. Microorganisms of the rhizosphere may comprise but are not limited to one or more of the following: Alternaria altemata, Alternaria brassicicola, Alternaria longipes, Alternaria solani, Ascochyta rabiei, Ashbya gossypii (Eremothecium gossypii), Aspergillus carbonarius, Aspergillus flavus, Aspergillus ruber, Bipolaris maydis, Bipolaris oryzae, Bipolaris sorokiniana, Bipolaris victoriae, Bipolaris zeicola, Blumeria graminis, Botrytis cinerea, Bremia lactucae, Ceraceosorus bombacis, Ceratocystis fimbriata, Ceratocystis harringtonii, Cercospora apii, Cercospora beticola, Cercospora fijiensis (Paracercospora fijiensis), Cercospora kikuchii, Cercospora nicotianae, Choanephora cucurbitarum, Claviceps purpurea, Colletotrichum acutatum, Colletotrichum coccodes, Colletotrichum fioriniae, Colletotrichum graminicola, Colletotrichum higginsianum, Colletotrichum incanum, Colletotrichum lentis, Colletotrichum lindemuthianum, Colletotrichum nymphaeae, Colletotrichum orbiculare, Colletotrichum salicis, Colletotrichum simmondsii, Colletotrichum sublineola, Colletotrichum trifolii, Colletrotrichum gloeosporioides, Cryphonectria parasitica, Diaporthe helianthi, Dothistroma septosporum, Erysiphe necator, Eutypa lata, Fulvia fulva, Fusarium culmorum, Fusarium fujikuroi, Fusarium graminearum, Fusarium langsethiae, Fusarium oxysporum, Fusarium poae, Fusarium pseudograminearum, Fusarium sambucinum, Fusarium solani, Fusarium verticillioides, Fusarium virguliforme, Geotrichum candidum, Gloeophyllum trabeum, Grosmannia clavigera, Heterobasidion annosum, Heterobasidion irregulare, Heterobasidion parviporum, Hyaloperonospora arabidopsidis, Kabatiella zeae (Aureobasidium zeae), Leptosphaeria biglobosa, Leptosphaeria maculans, Macrophomina phaseolina, Magnaporthe oryzae (Pyricularia oryzae), Magnaporthe poae, Marssonina brunnea (Drepanopeziza brunnea), Melampsora larici-populina, Microbotryum lychnidis-dioicae, Microbotryum silenes-dioicae, Microbotryum violaceum, Microdochium bolleyi, Mixia osmundae, Monilinia fructicola, Moniliophthora pemiciosa, Moniliophthora roreri, Mycosphaerella eumusae (Pseudocercospora eumusae), Neofusicoccum parvum, Neonectria ditissima, Ophiostoma novo-ulmi, Penicillium digitatum, Penicillium expansum, Peronospora tabacina, Phaeosphaeria nodorum (Parastagonospora nodorun), Phanerochaete camosa, Phyllachora maydis, Phytophthora cactorum, Phytophthora capsici, Phytophthora infestans, Phytophthora parasitica, Phytophthora sojae, Pseudocercospora fijiensis, Pseudocercospora fuligena, Pseudocercospora musae, Pseudopyrenochaeta lycopersici, Puccinia graminis f. sp. tritici, Puccinia polysora, Puccinia sorghi, Puccinia striiformis, Puccinia triticina, Pyrenophora teres, Pyrenophora tritici-repentis, Rhizoctonia solani, Rosellinia necatrix, Sclerotinia borealis, Sclerotinia sclerotiorum, Sclerotinia sclerotiorum, Setosphaeria turcica (Exserohilum turcicum), Sphaerulina musiva, Sporisorium reilianum, Sporisorium scitamineum, Stemphylium lycopersici, Stereum hirsutum, Synchytrium endobioticum, Thielaviopsis punctulata, Togninia minima (Phaeoacremonium minimum), Trametes cinnabarina, Ustilaginoidea virens, Ustilago hordei, Ustilago maydis, Valsa mali, Verticillium alfalfae, Verticillium dahliae, Verticillium longisporum, Zymoseptoria ardabiliae, Zymoseptoria brevis, Zymoseptoria tritici, Ophiosphaerella herpotricha, Ophiosphaerella korrae, Ophiosphaerella narmari, Pythium volutum, Pythium aristosporum, Pythium arrhenomanes, Pythium ultimum, Pythium irregulare, Pythium aphanidermatum, Pythium vanterpoolii, Pythium phragmitis, Pythium plurisporium, Pythium pyrilobum, Pythium graminicola, Pythium myriotylum, Pythium torulosum, Pythium scleroteichum, Pythium dissotocum, Pythium diclinum, Pythium inflatum, and Pythium catenulatum.

In some embodiments, pathogenic species may include but are not limited to Agrobacterium tumefaciens, Agrobacterium vitis, Burkholderia glumae, Clavibacter michiganensis, Dickeya dadantii, Dickeya solani, Erwinia amylovora, Listeria monocytogenes, Lonsdalea quercina, Pantoea stewartii, Pectobacterium atrosepticum, Pectobacterium brasiliense, Pectobacterium carotovorum, Pectobacterium odoriferum, Pectobacterium versatile, Pectobacterium wasabiae, Pseudomonas amygdali, Pseudomonas cannabina, Pseudomonas cichorii, Pseudomonas savastanoi, Pseudomonas syringae, Pseudomonas tolaasii, Ralstonia solanacearum, Salmonella enterica, Streptomyces scabiei, Xanthomonas axonopodis Â pv. manihotis, Xanthomonas axonopodis, Xanthomonas citri, Xanthomonas campestris, Xanthomonas campestris, Xanthomonas euvesicatoria, Xanthomonas hortorum, Xanthomonas oryzae, Xanthomonas translucens, and Xylella fastidiosa.

In some embodiments, based on analysis of the rhizosphere, the rhizosphere can be supplemented with a treatment, for example a specific microorganism disclosed herein that provides for a specific function.

### Characterizing Microorganism

In some embodiments, the method of the present disclosure comprises characterizing microorganisms of the rhizosphere. In various embodiments, the method of the present disclosure includes determining nucleic acid sequence reads of genetic material in a sample of a rhizosphere. In some embodiments, the sample may comprise parts or a portion of a plant. In some embodiments, the portion of the plant may comprise one or more of the following: a root, a stem, a leaf, a seed, a flower, a bud, a fruit, a petiole, a bulb, a cone, a tendril, a rhizome, or a bract. In some embodiments, the root may comprise a radicle, a taproot, a fibrous root, an adventitious root, an aerial root, a lateral root, or a modified root. In some embodiments, the sample may comprise one or more microorganisms. In some embodiments, the one or more microorganisms may comprise one or more of a bacterium, an archaebacterium, an actinomycete, a fungus, an algal cell, a protozoan, a virus, a viroid, or a nematode. In some embodiments, the sample may comprise beneficial microorganisms. In some embodiments, beneficial microorganisms of the present disclosure may comprise but are not limited to one of more of the genera Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, or Streptomyces. Beneficial microorganisms may include but are not limited to Mycorrhizal Fungi, Rhizobium bacteria, Azotobacter bacteria, Azospirillum bacteria, Bacillus spp., Pseudomonas spp., Trichoderma spp, Actinomycetes, Lactic Acid Bacteria (LAB), Streptomyces spp., Entomopathogenic fungi, Rhizobium, Azospirillum, Azotobacter, Frankia, Mycorrhizal fungi (e.g., Glomus spp.), Trichoderma, Bacillus subtilis, Bacillus thuringiensis, Pseudomonas fluorescens, Pseudomonas putida, Streptomyces, Saccharomyces cerevisiae (brewer's yeast), Lactobacillus, Clostridium, Agrobacterium, Enterobacter, Nitrosomonas, Nitrobacter, Actinomycetes, Penicillium, Aspergillus, Lysobacter, Arbuscular mycorrhizal fungi (AMF), Burkholderia, Bradyrhizobium, Clavibacter, Methylobacterium, Comamonas, Rhodospirillum, Streptomyces griseus, Streptomyces avermitilis, Streptomyces coelicolor, Streptomyces hygroscopicus, Streptomyces venezuelae, Streptomyces antibioticus, Streptomyces albus, Streptomyces noursei, Streptomyces aureofaciens, Streptomyces roseosporus, Streptomyces lividans, Streptomyces parvulus, Streptomyces chattanoogensis, Streptomyces griseoluteus, Streptomyces nogalater, Streptomyces fradiae, Streptomyces erythraeus, Streptomyces tanashiensis, Streptomyces kanamyceticus, Streptomyces lydicus, Bacillus licheniformis, Bacillus amyloliquefaciens, Bacillus megaterium, Bacillus coagulans, Bacillus pumilus, Bacillus cereus, Bacillus polymyxa, Bacillus circulans, Bacillus firmus, Bacillus popilliae, Bacillus pasteurii, Bacillus macerans, Bacillus mucilaginosus, Bacillus azotofixans, Bacillus brevis, Bacillus stearothermophilus, Bacillus sphaericus, Bacillus acidocaldarius, Bacillus halodurans, Bacillus smithii, Bacillus marinus, Bacillus infemus, Bacillus alcalophilus, Bacillus psychrophilus, Bacillus halodenitrificans, Bacillus thermoglucosidasius, Bacillus thermoleovorans, Bacillus boroniphilus, Bacillus schlegelii, Bacillus proteolyticus, Bacillus thermocloacae, Bacillus thermoamylovorans, Bacillus amylolyticus, Bacillus lautus, Bacillus cohnii, Bacillus phycophilus, Bacillus chroococcidiopsidis, Bacillus methanolicus, Bacillus psychrotolerans, Bacillus azotoformans, Bacillus fluvialis, Bacillus lautus, Bacillus conterminus, Bacillus fusiformis, Bacillus korlensis, Bacillus tropicus, Bacillus arsenicoselenatis, Bacillus siralis, Bacillus ehimensis, Bacillus toyonensis, Bacillus nakamurai, and any combination thereof.

In some embodiments, the sample may comprise one or more of parts or a portion of a plant or seed, air, water, soil, paper, or a portion of the environment around a plant or seed. In some embodiments, the paper may comprise germination paper. A kit may be used to collect the sample. Components of a kit may comprise but are not limited to one or more of each of the following: soil microbiota traps, soil microbiota trap containers, field markers, coolers, cold packs, water, gloves, scoopulas, pipettes, dry ice, liquid nitrogen, refrigerators, soil, fertilizer, disinfectant, bottles, thermometers, pH strips, soil testing kits, water testing kits, and/or notepads.

In some embodiments, the sample can be processed to obtain genetic material. In some embodiments, the processed genetic material can be and sequenced. In some embodiments, the results of the sequencing can be analyzed to characterize the genetic material. In some embodiments, characterizing the genetic material may identify the species, genus, family, order, class, phylum, kingdom, or domain corresponding to said genetic material. In some embodiments, genetic material can be obtained from a seed, a plant, a microorganism, a fungus, an insect, or an animal. In some embodiments, genetic material can be deoxyribonucleic acid (DNA) or ribonucleic acid (RNA). The DNA may comprise genomic DNA (gDNA), mitochondrial DNA (mtDNA), or circular DNA. The RNA may comprise ribosomal RNA (rRNA), small nuclear RNA (snRNA), messenger RNA (mRNA), transfer RNA (tRNA), small nucleolar RNA (snoRNA), micro RNA (miRNA), or long noncoding RNA (lncRNA). In some embodiments, the genetic material comprises a denatured DNA molecule or fragment thereof. In some embodiments, the genetic material comprises DNA selected from: genomic DNA, viral DNA, mitochondrial DNA, plasmid DNA, amplified DNA, circular DNA, circulating DNA, cell-free DNA, or exosomal DNA. In some embodiments, the DNA is single-stranded DNA (ssDNA), double-stranded DNA, denaturing double-stranded DNA, synthetic DNA, and combinations thereof. In some embodiments, the circular DNA may be cleaved or fragmented.

In some embodiments, the RNA comprises fragmented RNA. In some embodiments, the RNA comprises partially degraded RNA. In some embodiments, the RNA comprises a microRNA or portion thereof. In some embodiments, the RNA comprises an RNA molecule or a fragmented RNA molecule (RNA fragments) selected from: a microRNA (miRNA or miR), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, or combinations thereof.

In some embodiments, the sequencing performed on the genetic material can be DNA sequencing, RNA sequencing, metagenomics sequencing, whole genome sequencing, whole exome sequencing, whole transcriptome sequencing, methylation sequencing, skim sequencing, or chromatin sequencing. In some embodiments, the sequencing can comprise next generation sequencing (NGS). In some embodiments, the sequencing can comprise Sanger sequencing, Solexa-Illumina sequencing, Ligation-based sequencing (SOLiD™), pyrosequencing, strobe sequencing (SMRT™) or semiconductor array sequencing (Ion Torrent™). In some embodiments, the sequencing is metagenomic sequencing. In some embodiments, the sequencing can comprise shotgun sequencing.

In some embodiments, the exact concentrations of nucleic acids collected using procedures described herein may be quantified. The quantification of the nucleic acids may allow for more accurate normalization of DNA libraries generated during sequencing. A fluorometer may be used to quantify the concentrations of nucleic acids according to standard protocols or variations thereof.

In some embodiments, the method further includes determining, by analyzing the nucleic acid sequence reads, a first set of measures of a plurality of gene functions represented in the nucleic acid sequence reads. In some embodiments, the method comprises determining, by further analyzing the plurality of gene functions, a second set of measures of a plurality of metabolic pathways of microorganisms present in soil or in an environment at a geographical location of a sample collection. In some embodiments, the geographical location can be a farm, ranch, greenhouse, laboratory, controlled setting, golf course or any other soil environment. In some embodiments, the method comprises determining, by processing the plurality of metabolic pathways, a third set of measures of a plurality of soil health indicators of the soil or environment at the geographical location of sample collection. In some embodiments, the plurality of soil or environment health indicators can include but are not limited to a plurality of levels of granularity. In some embodiments, the method comprises determining a measure of an agronomic attribute of the soil or environment at the geographical location of the sample collection as a function of the first, second, or third sets of measures. In some embodiments, the measure of an agronomic attribute of the soil is transmitted to a client device for display.

In some embodiments, the plurality of gene functions may include one or more of nitrogen cycling, phosphorous cycling, carbon cycling, drought resistance, polymer degradation, or oxygen availability. In some embodiments, the agronomic attribute comprises nitrogen mineralization, plant height, tillering capacity, root length and mass, grain size and weight, ability to resists pests, diseases and environmental stressors. In some embodiments, the plurality of metabolic pathways comprises secreted proteases. In some embodiments, the plurality of metabolic pathways comprises urea mineralization, the TCA cycle, glyoxylate, dicarboxylate metabolism, aromatic amino acid (phenylalanine, tryptophan, and tyrosine) biosynthesis or the phenylpropanoid biosynthesis pathway. In some embodiments, a metabolic pathway can be determined by measuring a constituent gene. Metabolic pathways may include but are not limited to drought resistance pathways, exopolysaccharide (EPS) biosynthesis pathways, trehalose biosynthesis pathways, osmoprotectant biosynthesis pathways, ethylene breakdown pathways, auxin biosynthesis pathways, pyruvate metabolism pathways, phosphorous cycling pathways, pentose phosphate pathways, phosphotransferase pathways, oxidative phosphorylation pathways, phosphonate and phosphinate metabolism pathways, phosphotransferase system pathways, two-component system pathways, transporter pathways, organic phosphoester hydrolysis pathways, purine metabolism pathways, pyrimidine metabolism pathways, nitrification pathways, nitrogen cycling pathways, denitrification pathways, assimilatory nitrate reduction pathways, dissimilatory nitrate reduction pathways, nitrogen fixation pathways, anammox pathways, organic degradation and synthesis pathways, carbohydrate degradation pathways, carbohydrate binding molecule pathways, carbohydrate esterase pathways, cellulosome pathways, glycoside hydrolase pathways, glycosyltransferase pathways, polysaccharide lyase pathways, cellulosome pathways, pentose phosphate pathways, organic phosphoester hydrolysis pathways, assimilatory nitrate reduction pathways, dissimilatory nitrate reduction pathways, organic degradation and synthesis pathways, carbohydrate esterase pathways, glycoside hydrolase pathways, polysaccharide lyase pathways, immobilization gene pathways, mineralization gene pathways, sugar pathways, polymer pathways, aromatic pathways, sulfur metabolism pathways, methanogen pathways, ammonification pathways, ethylene breakdown pathways, scavenging pathways, manganese-dependent inorganic phosphatase pathways, exopolyphosphatase pathways, and any combination thereof. Constituent genes of metabolic pathways of interest disclosed herein include but are not limited to cysE, wecA, wecG, wcaI, wcaJ, wcaF, wcaB, pgaC, pgaD, wecF, gumD, gumH, gumI, gumK, gumM, gumF, gumG, gumL, wcaE, wcaC, exoO, exoM, exoA, exoL, exoH, exoW, exoV, exoU, exoY, exoZ, amsB, amsD, amsE, wcaL, amsG, wcaK, alg8, alg44, algX, algI, algJ, algF, vpsD, vpsI, pslA, pslF, pslH, pslI, pssM, icaD, pslC, wcaA, vpsK, aceA, aceB, aceQ, aceP, aceR, aceI, pssA, pssD, pssE, pssC, pssS, pssF, pssI, pssG, pssH, pssJ, pssR, pssK, cpsG, cpsF, otsA, otsB, treM, treP, treT, treY, treZ, betA, ectA, ectB, ectC, proA, proB, accD, iaaM, iaaH, pps, ppdK, pyk, pckG, ppc, pckA, gdh, gcd, gnl, gntK, gnd, rpiA, prsA, deoB, ptsI, ptsH, ppk, ppa, pepM, pphA, ppd, phnX, fomC, phpC, mpnS, phnG, phnH, phnI, phnK, phnL, phnM, phnJ, phnP, phnN, phnPP, phny, phnA, phnW, phnO, pbfA, phnY, phnZ, phoU, phoR, phoB, phoP, SenX3, RegX3, pgtC, pgtB, pgtA, pgtP, pstS, pstC, pstA, pstB, pit, htxB, ptxA, ptxB, ptxC, phnD_phosphite, phnD, phnE, phnC, ugpB, ugpA, ugpE, ugpC, phnS, phnV, phnU, phnT, glpT, aepX, aepV, aepW, aepP, aepS, opd, pafA, phoA, phoD, phoX, phoN, aphA, phoC, olpA, phy, appA, ugpQ, glpQ, htxA, ptxD, lysR, phnR, phnF, phoH, purF, purD, purN, purT, purL, purS, purQ, purM, purK, purE, ADE2, purC, purB, purH, purP, purO, guaB, guaA, gmk, ushA, ndk, spoT, ppx, purA, adk, pyrE, pyrF, ushA, cmk, pyrH, ndk, pyrG, rtpR, nrdD, nrdA, nrdE, nrdB, nrdF, nrdJ, dcd, dut, thyA, tmk, amoA_A, amoB_A, amoC_A, amoA_B, amoB_B, amoC_B, hao, nxrA, nxrB, napA, napB, napC, narG, narH, narJ, narI, nirK, nirS, norB, norC, nosZ, narZ, narY, narV, narW, nasA, nasB, nirA, NR, narB, narC, napA, napB, napC, narG, narH, narJ, nar, narZ, narY, narV, narW, nirB, nirD, nrfA, nrfB, nrfC, nrfD, anfG, nifD, nifH, nifK, nifW, hzo, hzsA, hzsB, hzsC, hdh, ureA, ureB, ureC, nao, nmo, gdh_K00260, gdh_K00261, gdh_K00262, gdh_K15371, gs_K00264, gs_K00265, gs_K00266, gs_K00284, glsA, glnA, asnB, ansB, hcp, pmoA, pmoB, pmoC, AA0, AA1, AA10, AA2, AA3, AA4, AA5, AA6, AA7, AA8, AA9, CBM0, CBM1, CBM10, CBM11, CBM12, CBM13, CBM14, CBM15, CBM16, CBM17, CBM18, CBM19, CBM2, CBM20, CBM21, CBM22, CBM23, CBM24, CBM25, CBM26, CBM27, CBM28, CBM29, CBM3, CBM30, CBM31, CBM32, CBM33, CBM34, CBM35, CBM36, CBM37, CBM38, CBM39, CBM4, CBM40, CBM41, CBM42, CBM43, CBM44, CBM45, CBM46, CBM47, CBM48, CBM49, CBM5, CBM50, CBM51, CBM52, CBM53, CBM54, CBM55, CBM56, CBM57, CBM58, CBM59, CBM6, CBM60, CBM61, CBM62, CBM63, CBM64, CBM65, CBM66, CBM67, CBM7, CBM8, CBM9, CE0, CE1, CE10, CE11, CE12, CE13, CE14, CE15, CE16, CE2, CE3, CE4, CE5, CE6, CE7, CE8, CE9, cohesin, dockerin, GH0, GH1, GH10, GH100, GH101, GH102, GH103, GH104, GH105, GH106, GH107, GH108, GH109, GH11, GH110, GH111, GH112, GH113, GH114, GH115, GH116, GH117, GH118, GH119, GH12, GH120, GH121, GH122, GH123, GH124, GH125, GH126, GH127, GH128, GH129, GH13, GH130, GH131, GH132, GH14, GH15, GH16, GH17, GH18, GH19, GH2, GH20, GH21, GH22, GH23, GH24, GH25, GH26, GH27, GH28, GH29, GH3, GH30, GH31, GH32, GH33, GH34, GH35, GH36, GH37, GH38, GH39, GH4, GH40, GH41, GH42, GH43, GH44, GH45, GH46, GH47, GH48, GH49, GH5, GH50, GH51, GH52, GH53, GH54, GH55, GH56, GH57, GH58, GH59, GH6, GH60, GH61, GH62, GH63, GH64, GH65, GH66, GH67, GH68, GH69, GH7, GH70, GH71, GH72, GH73, GH74, GH75, GH76, GH77, GH78, GH79, GH8, GH80, GH81, GH82, GH83, GH84, GH85, GH86, GH87, GH88, GH89, GH9, GH90, GH91, GH92, GH93, GH94, GH95, GH96, GH97, GH98, GH99, GT0, GT1, GT10, GT11, GT12, GT13, GT14, GT15, GT16, GT17, GT18, GT19, GT2, GT20, GT21, GT22, GT23, GT24, GT25, GT26, GT27, GT28, GT29, GT3, GT30, GT31, GT32, GT33, GT34, GT35, GT36, GT37, GT38, GT39, GT4, GT40, GT41, GT42, GT43, GT44, GT45, GT46, GT47, GT48, GT49, GT5, GT50, GT51, GT52, GT53, GT54, GT55, GT56, GT57, GT58, GT59, GT6, GT60, GT61, GT62, GT63, GT64, GT65, GT66, GT67, GT68, GT69, GT7, GT70, GT71, GT72, GT73, GT74, GT75, GT76, GT77, GT78, GT79, GT8, GT80, GT81, GT82, GT83, GT84, GT85, GT86, GT87, GT88, GT89, GT9, GT90, GT91, GT92, GT93, GT94, PL0, PL1, PL10, PL11, PL12, PL13, PL14, PL15, PL16, PL17, PL18, PL19, PL2, PL20, PL21, PL22, PL3, PL4, PL5, PL6, PL7, PL8, PL9, SLH, manganese-dependent inorganic pyrophosphatase, exopolyphosphatase, hypophosphite dioxygenase, phosphonate dehydrogenase, diaminopimelate decarboxylase, transcriptional regulator of 2-aminoethylphosphonate degradation operons, GntR family transcriptional regulator, phosphonate transport system regulatory protein, phosphate starvation-inducible protein, hydroxylamine reductase, methane/ammonia monooxygenase subunit A, methane/ammonia monooxygenase subunit B, and methane/ammonia monooxygenase subunit C.

In some embodiment, genes of interest identified through the methods disclosed herein include but are not limited to nitrification genes, denitrification genes, nitrogen fixation genes, phosphorous solubilization genes, iron acquisition genes, carbon fixation genes, organic nitrogen breakdown genes, organic carbon breakdown genes, low oxygen environment genes, anoxic environment genes, high oxygen environment genes, stress adaptation genes, methanogenesis genes, sulfur oxidation genes (sulfide, thiosulfate, sulfite, etc.), sulfur reduction genes (APS, sulfite, sulfur, etc.), calcium transport genes, nodulating bacteria genes, potassium solubilization genes, and any combination thereof. Genes of interest may include deaminase genes, ligninase genes, glucoamylase genes, pectinase genes, endoglucanase genes, cellulase genes, cellobiose-phosphorylase genes, beta-agarase genes, lysozyme genes, chitinase genes, phytase genes, and any combination thereof.

In some embodiments, the method comprises determining a recommendation for treatment of the soil or environment at the geographical location of sample collection. In some embodiments, the recommendation for treatment of the soil or environment at the geographical location of sample collection can use at least the measure of the agronomic attribute. In some embodiments, the soil or environment at the geographical location of sample collection can be treated to improve nitrogen mineralization, plant height, tillering capacity, root length and mass, grain size and weight, ability to resists pests, diseases and environmental stressors. In some embodiments, the method comprises filtering out at least one metabolic pathway of the plurality of metabolic pathways. In some embodiments, the soil or environment at the geographical location of sample collection can be treated to improve a metabolic pathway.

In some embodiments, the method of the present disclosure comprises determining a beneficial plant management technique. In some embodiments, the beneficial plant management technique can be determined based on the results of the sequencing of genetic material obtained from a sample of a rhizosphere. In some embodiments, the beneficial plant management technique for a plant improves a plant characteristic. One or more beneficial plant management techniques may be recommended. One or more beneficial plant management techniques may be utilized at the same time. Beneficial plant management techniques may be applied to different areas of a field. Beneficial plant management techniques may be applied at different times. In some embodiments, the technique is based at least on bioinformatic analysis of the results of said sequencing. In some embodiments, the plant characteristic comprises plant yield. In some embodiments, the plant characteristic comprises plant health. In some embodiments, the plant characteristic comprises root length and mass, root system architecture, grain size and weight, or ability to resists pests, diseases and environmental stressors. In some embodiments, the beneficial plant management technique may comprise identifying a beneficial growth temperature, humidity range, soil composition, soil treatment composition, nutrient composition, plant food, biofertilizer, biostimulant, or any combination thereof. In some embodiments, the soil treatment composition may comprises one or more of Trichoderma harzianum, Bacillus amyloliquefaciens, Bacillus subtilis, Myxococcus xanthus, Wickerhamomyces anomalus, Azotobacter vinelandii, Frateuria aurantia, Pseudomonas chlororaphis, Starmerella bombicola, Saccharomyces boulardii, Pichia occidentalis, Pichia kudriavzevii, or Meyerozyma guilliermondii. In some embodiments, the biostimulant can provide a benefit to a plant. In some embodiments, the benefit may comprise but is not limited to one or more of increase growth, increase disease resistance, increase nutrition efficiency, increase stress tolerance, or improved crop quality. In some embodiments, the biofertilizer may comprise microbes. In some embodiments, the microbes may comprise but are not limited to one or more of the genera: Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, or Streptomyces.

In some embodiments, a beneficial plant management technique may be determined by calculation of a score or total score as disclosed herein. In some embodiments, a beneficial plant management technique may cause a change in a score or a total score as disclosed herein. Said score may take into account one or more factors selected from observed genera, community evenness, mycorrhizae, fungal/bacteria ratio (F/B ratio), rare biosphere, nitrification, denitrification, nitrogen fixation, phosphorous cycling, polymer degradation, drought resistance, disturbance, total score, and any combination thereof. In some embodiments, analysis of genes disclosed in Tables 1-32 may be used to generate said score.

In some embodiments, disease may comprise but is not limited to Damping-Off, Verticillium Wilt, Root-Knot Nematode, Anthracnose, Crown Gall, Fusarium Wilt, Charcoal Rot, Root Rot, Armillaria Root Rot, Phytophthora Root Rot, Phytophthora Crown and Root Rot, White Mold, Pythium Root Rot, Pythium, Fusarium Root Rot, Rhizoctonia Root Rot, Black Root Rot, Phytophthora Root and Crown Rot, Bacterial Leaf Spot, Seedling Blight, Bacterial Blight, Dry Rot, Rhizoctonia, Alternaria Rot, Sudden Wilt, Kernel Mold, Bacterial Canker, Fusarium Root and Stem Rot, Rhizoctonia Solani Anastomosis Group 3, Fusarium Rot, Leaf Spot, Bacterial Soft Rot, Ripe Fruit Rot, Botrytis Gray Mold, Wood-Decay Fungi, Brown Spot, Phytophthora Crown Rot, Clubroot, Seed Rot, Fusarium Blight, Armillaria Root Rot (Oak Root Fungus), Take-All, Alternaria Blight, Black Dot, Aphanomyces Root Rot, Black Foot Disease, Crown Rot, Bottom Rot, Downy Mildew, Bacterial Black Rot, Dry Root Rot, Rootlet Rot, Esca and Petri Disease, Soybean Cyst Nematode, Eutypa Dieback, Head Blight, Fusarium Basal Rot, Melon Vine Decline, Black Rot, Phytophthora Fruit and Crown Rot, Fusarium Head Blight, Pythium Seed and Seedling Rot, Botrytis Fruit Rot, Root Dieback, Ascochyta Blight, Sclerotinia Stem Rot, Bacterial Wilt, Seedling Blight and Root Rot, Fusarium Yellows, Stem Canker, Gray Mold, Common Root Rot, Gray Mold Rot, White Rot, Covered Smut, Bending Head, Red Stele, Crater Rot, Southern Root-Knot Nematode, Crown Blight, Powdery Scab, Black Mold Rot/Black Shoulder, Rhizopus Soft Rot, Black Point, Common Bunt (Stinking Smut), Damping Off, Sulfur Fungus, Apical Chlorosis, Pink Rot, Damping-Off and Fruit Rot, Pythium Leak, Dothiorella Gummosis, Bacterial Head Rot, Alternata Blight, Root and Stem Rot, Basal Glume Rot, Sclerotinia Wilt (White Mold), Alternaria Late Blight, Soreshin, Early Blight, Stewart's Wilt, Enterobacter Bulb Decay, Varnish Spot, Ergot, Phytophthora Shuck and Kernel Rot, Black Scurf, Pod and Stem Blight, Esca Disease, Alternaria Black Spot, Blackleg, Canker, Fire Blight, Rhizoctonia Root and Crown Rot, Fusarium, Bacterial Kernel Blight, Blossom Blast, Center Rot, Blossom Blight, Alternaria Stem Blight, Fusarium Canker, Sclerotinia Rot, Fusarium Crown and Root Rot, Seed Rot and Seedling Blight, Botrytis Blight, Silver Scurf, Botrytis Blossom Blight, Southern Bacterial Wilt, Botrytis Blossom Blight and Fruit Rot, Common Smut, Botrytis Bunch Rot, Sudden Death Syndrome, Alternaria, Trichoderma Foot Rot, Fusarium Stalk Rot, Cottony Rot, Ashy stem blight, Phytophthora Rot, Botrytis Diseases And Disorders, Pineapple Disease, Goss' Wilt, Plum Pocket, Grapevine Trunk Disease, Potato Scab, Gray Leaf Spot, Punky Rot, Botrytis Fruit Blight, Pythium Blight, Basal Stem Blight, Alternaria Spot, Hairy Roots, Pythium Wilt, Hairy Turkey Tail, Bacterial Gall, Bean Blight, Rhizoctonia Root Canker, Head Rot, Rhizoctonia Seedling Blight, Bacterial Spot, Rhizoctonia Stem Canker, White Rust, Rice Blast, Jacket Rot, Root and Crown Rot, Aspergillus Crown Rot, Cercospora Leaf Spot, Leaf Scald, Bacterial Leaf Streak, Botrytis Neck Rot, Sclerotinia, Lettuce Drop, Bacterial Twig Dieback, Bean Rust, Colletotrichum Leaf Spot, Mucor Rot, Bacterial Leafspot, Northern Root-Knot Nematode, Sheath Rot, Peach Leaf Curl, Soft Rot, Peach Root-Knot Nematode, Sour Skin, Penicillium Rot, Southern Blight, Phoma Basal Rot, Banded Sclerotial Leaf Disease, Phytophthora, Stem Rot, Aspergillus Rot, Striatura Ulcerosa, Phytophthora Crown and Spear Rot, Bacterial Mosaic, Brown Blight, Common Split Gill, Brown Patch, Turkey Tail, Phytophthora Neck and Bulb Rot, Verticillium, Belaat, Verticillum Wilt, Phytophthora Root and Stem Rot, Bacterial Blast (Citrus Blast), Inflorescence Rot, Bacterial Streak, Jacket and Ripe Fruit Rot, and Fusarium Seed Rot.

In some embodiments, the plant may exhibit an improved phenotype of agronomic interest following the implantation of a beneficial plant management technique. In some embodiments, the phenotype of agronomic interest may comprise but is not limited to: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increased yield, increased yield under water-limited conditions, health enhancement, vigor improvement, growth improvement, photosynthetic capability improvement, nutrition enhancement, altered protein content, altered oil content, increased biomass, increased shoot length, increased root length, improved root architecture, increased seed weight, altered seed carbohydrate composition, altered seed oil composition, altered number of pods, delayed senescence, stay-green, or altered seed protein composition.

In some embodiments, the present disclosure provides a method of typing a microbiome for having a desirable or undesirable signature. In some embodiments, the method of typing a microbiome can comprise analyzing the composition of the population of microorganisms in said microbiome. In some embodiments, the composition of the population of microorganisms in said microbiome can be determined based on taxonomic variation in the DNA sequence of the microbial 16S-23S rRNA internal transcribed spacer (ITS) regions in the genomic DNA of said microorganisms. In some embodiments, the composition of the population of microorganisms in said microbiome is determined based on taxonomic variation in the DNA sequence of the microbial 16S regions in the genomic DNA of said microorganisms. In some embodiments, primers can be designed for regions in the 16S that comprise conserved regions of DNA. In some embodiments, primers can be designed for regions in the 23S that comprise conserved regions of DNA. In some embodiments, primers can be designed for regions in the ITS that comprise conserved regions of DNA. In some embodiments, primers can be designed for regions in the 16S and 23S rRNA sequences flanking said ITS region that comprise conserved regions of DNA. In some embodiments, primers can amplify the ITS regions. In some embodiments, primers can amplify the 16S regions. In some embodiments, primers can amplify the 23S regions. In some embodiments, said analyzing can comprise: a) providing a sample of genomic DNA from the microorganisms in a microbiome; b) performing a PCR amplification reaction on said sample of genomic DNA using at least one set of PCR amplification primers directed to said conserved DNA regions to thereby amplify and provide amplification products of said target regions comprised in said genomic DNA sample; c) analyzing said amplification products based on length differences in said amplification products to thereby provide a test signature of the composition of the population of microorganisms in said microbiome; d) comparing said test signature with at least one reference signature of a desirable microbiome and/or with at least one reference signature of an undesirable microbiome, preferably by clustering one or more of ITS, 16S, and 23S profiles, and classifying the test signature as a signature of a desirable microbiome or as a signature of an undesirable microbiome.

In some embodiments, the present disclosure provides a method of analyzing the composition of a microbiome based on taxonomic variation in the DNA sequence of the microbial 16S-23S rRNA internal transcribed spacer (ITS), 16S, 23S, and/or ITS regions in the genomic DNA of the microorganisms in said microbiome, wherein the sequences of conserved DNA regions comprised in the 16S and 23S rRNA sequences flanking said ITS region in the genomic DNA of said microorganisms comprise primer binding sites for amplification of said ITS regions, said method comprising the steps of: a) providing a sample of genomic DNA from a microbiome; b) providing a PCR calibrator system, comprising a set of PCR amplification primers wherein at least one of which primers comprises a label, and a set of at least two PCR calibrators, each PCR calibrator comprising a DNA fragment comprising a spacer region having a DNA sequence of a given length flanked by upstream and downstream adapter DNA sequences that comprise primer binding sites for binding of said PCR amplification primers wherein said set of PCR amplification primers is for PCR amplifying the spacer region DNA sequence of all PCR calibrators in said set of at least two PCR calibrators. In some embodiments, the spacer region DNA sequence comprised in each of said PCR calibrators in said set of at least two PCR calibrators is of a different length. In some embodiments, each PCR calibrator in said set of at least two PCR calibrators is present in equal amount or in a known amount relative to other PCR calibrators in said set; c) adding said set of at least two PCR calibrators from said PCR calibrator system to said sample of genomic DNA; d) performing a PCR amplification reaction on said sample of genomic DNA comprising said set of at least two PCR calibrators using said set of PCR amplification primers from said PCR calibrator system as a first set of amplification primers to amplify and provide amplification products of said ITS region(s) comprised in said set of at least two PCR calibrators. In some embodiment, the method comprises using at least a second set of PCR amplification primers directed to said flanking conserved DNA regions to thereby co-amplify and provide amplification products of said ITS regions comprised in said sample of genomic DNA; e) providing a standard curve by determining the PCR amplification efficiency of each of said at least two PCR calibrators from said PCR calibrator system in said PCR amplification reaction of step d) and expressing said PCR amplification efficiency as a function of the length of the DNA sequence of the ITS region.

In some embodiments, the method further comprises f) determining the length-specific amplification efficiency for ITS regions of different length comprised in said genomic DNA sample and amplified in step d) using the standard curve as provided in step e); g) determining the abundance of microbial 16S-23S rRNA internal transcribed spacer (ITS) regions of different length in said microbiome using the length-specific amplification efficiencies determined in step f), and h) analyzing the composition of a population of microorganisms based on the abundances of ITS regions of different length determined in step g). In some embodiments of the above method, said standard curve is based on at least five PCR calibrators of different length ranging in length from 50 to 1200 bps.

### Gene Analysis

In some embodiments, genes can be analyzed from the rhizomicrobiome. In some embodiments, genes can be analyzed through transcriptomics, microarray, qPCR, or semi-quantitative PCR. In some embodiments, the genes analyzed from the rhizobiome are associated with one or more microorganisms. Non-limiting examples of microorganisms include but are not limited to bacteria, fungi, virus, amoeba, and algae. In some embodiments, the genes analyzed from the rhizobiome are associated with plants. Non-limiting examples of plants include but are not limited to a crop plant, a non-crop plant, a cultivated plant, a non-cultivated plant, an open pollinated plant, a non-hybrid plant, an heirloom plant, a hybrid plant, a genetically modified plant, a monocot plant, and a dicot plant. In some embodiments, the genes analyzed from the rhizobiome are associated with plant roots. In some embodiments, the genes analyzed from the rhizobiome are associated with one or more parts of a plant root. Non-limiting examples of parts of plant roots include but are not limited to a radicle, a taproot, a fibrous root, an adventitious root, an aerial root, a lateral root, and a modified root. In some embodiments, the genes analyzed from the rhizobiome are associated with root nodules. In some embodiments, the genes analyzed from the rhizobiome are associated with nitrogen-fixing root nodules. In some embodiments, the genes analyzed from the rhizobiome are associated with one or more microorganisms. In some embodiments, the genes correspond to a protein. Non-limiting examples of proteins include but are not limited to fibrous proteins, globular proteins, enzymes, structural proteins, nutrient proteins, regulatory proteins, defense proteins, transport proteins, storage proteins, contractile proteins, and toxic proteins. In some embodiments, the protein comprises an enzyme. Non-limiting examples of enzymes include but are not limited to transferases, ligases, oxidoreductases, isomerases, hydrolases, and lyases.

In some embodiments, about 1, 10, 20, 30, 40, 50, 100, 200, 300, 500, 1000, 1500, 2000, 2500, 3000, 3500, 4000, 4500, 5000, 5500, 6000, 6500, 7000, 7500, to about 8000 genes are analyzed from the rhizobiome. In some embodiments, less than about 10, 20, 30, 40, 50, 100, 200, 300, 500, 1000, 1500, 2000, 2500, 3000, 3500, 4000, 4500, 5000, 5500, 6000, 6500, 7000, 7500 or 8000 genes are analyzed from the rhizobiome. In some embodiments, greater than about 10, 20, 30, 40, 50, 100, 200, 300, 500, 1000, 1500, 2000, 2500, 3000, 3500, 4000, 4500, 5000, 5500, 6000, 6500, 7000, 7500 or 8000 genes are analyzed from the rhizobiome.

In some embodiments, one or more genes are detected in the rhizobiome. In some embodiments, the transcripts of one or more genes is increased as compared to a control sample or following treatment of the environment from which the sample was collected. In some embodiments, the expression of one or more genes is increased as compared to a control sample or following treatment of the environment from which the sample was collected. In some embodiments, the transcripts of one or more genes is decreased as compared to a control sample or following treatment of the environment from which the sample was collected. In some embodiments, the expression of one or more genes is decreased as compared to a control sample or following treatment of the environment from which the sample was collected.

In some embodiments, the one or more genes are associated with exopolysaccharide (EPS) Biosynthesis (Table 1). In some embodiments, the one or more genes are associate with trehalose biosynthesis (Table 2). In some embodiments, the one or more genes are associated with osmoprotectant biosynthesis (Table 3). In some embodiments, the one or more genes are associated with pyruvate metabolism (Table 4). In some embodiments, the one or more genes are associated with the pentose phosphate pathway (Table 5). In some embodiments, the one or more genes are associated with phosphonate and phosphinate metabolism (Table 6). In some embodiments, the one or more genes are associated with the two-component system (Table 7). In some embodiments, the one or more genes are associated with transporters (Table 8). In some embodiments, the one or more genes are associated with organic phosphoester hydrolysis (Table 9). In some embodiments, the one or more genes are associated with purine metabolism (Table 10). In some embodiments, the one or more genes are associated with pyrimidine metabolism (Table 11). In some embodiments, the one or more genes are associated with nitrification (Table 12). In some embodiments, the one or more genes are associated with denitrification (Table 13). In some embodiments, the one or more genes are associated with assimilatory nitrate reduction (Table 14). In some embodiments, the one or more genes are associated with dissimilatory nitrate reduction (Table 15). In some embodiments, the one or more genes are associated with nitrogen fixation (Table 16). In some embodiments, the one or more genes are associated with anammox (Table 17). In some embodiments, the one or more genes are associated with organic degradation and synthesis (Table 18). In some embodiments, the one or more genes are associated with auxiliary activities (Table 19). In some embodiments, the one or more genes are associated with carbohydrate binding modules (Table 20). In some embodiments, the one or more genes are associated with carbohydrate esterases (Table 21). In some embodiments, the one or more genes are associated with glycoside hydrolases (Table 22). In some embodiments, the one or more genes are associated with glycosyltransferases (Table 23). In some embodiments, the one or more genes are associated with polysaccharide lyases (Table 24). In some embodiments, the one or more genes are associated with immobilization (Table 25). In some embodiments, the one or more genes are associated with mineralization (Table 26). In some embodiments, the one or more genes are associated with sugars (Table 27). In some embodiments, the one or more genes are associated with polymers (Table 28). In some embodiments, the one or more genes are associated with aromatics (Table 29). In some embodiments, the one or more genes are associated with sulfur metabolism (Table 30). In some embodiments, the one or more genes are associated with methanogen (Table 31). In some embodiments, the one or more genes are associated with ethylene breakdown (Table 32). In some embodiments, the one or more genes are associated with auxin biosynthesis (Table 32). In some embodiments, the one or more genes are associated with phosphotransferase system (Table 32). In some embodiments, the one or more genes are associated with oxidative phosphorylation (Table 32). In some embodiments, the one or more genes are associated with cellulosome (Table 32). In some embodiments, the one or more genes are associated with ammonification (Table 32). In some embodiments, the one or more genes are associated with a rieske iron-sulfur protein (Table 32). In some embodiments, the one or more genes are associated with cytochrome b-561 (Table 32). In some embodiments, the one or more genes are associated with manganese-dependent inorganic pyrophosphatase (Table 32). In some embodiments, the one or more genes are associated with exopolyphosphatase (Table 32). In some embodiments, the one or more genes are associated with exopolyphosphatase (Table 32). In some embodiments, the one or more genes are associated with hypophosphite dioxygenase (Table 32). In some embodiments, the one or more genes are associated with phosphonate dehydrogenase (Table 32). In some embodiments, the one or more genes are associated with diaminopimelate decarboxylase (Table 32). In some embodiments, the one or more genes are associated with transcriptional regulator. In some embodiments, the one or more genes are associated with transcriptional regulator of 2-aminoethylphosphonate degradation operons (Table 32). In some embodiments, the one or more genes are associated with phosphonate transport system regulatory protein (Table 32). In some embodiments, the one or more genes are associated with phosphate starvation-inducible protein (Table 32). In some embodiments, the one or more genes are associated with hydroxylamine reductase (Table 32). In some embodiments, the one or more genes are associated with methane/ammonia monooxygenase. In some embodiments, the one or more genes are associated with methane/ammonia monooxygenase A (Table 32). In some embodiments, the one or more genes are associated with methane/ammonia monooxygenase B (Table 32). In some embodiments, the one or more genes are associated with methane/ammonia monooxygenase C (Table 32).

TABLE 1
Exopolysaccharide (EPS) Biosynthesis Genes

| Gene | Gene ID | Gene | Gene ID | Gene | Gene ID |
|---|---|---|---|---|---|
| cysE | K00640 | exoW | K16562 | icaD | K21461 |
| wecA | K02851 | exoV | K16563 | pslC | K25205 |
| wecG | K02852 | exoU | K16564 | wcaA | K25875 |
| wcaI | K03208 | exoY | K16566 | vpsK | K25886 |
| wcaJ | K03606 | exoZ | K16568 | aceA | K25887 |
| wcaF | K03818 | amsB | K16700 | aceB | K25888 |
| wcaB | K03819 | amsD | K16701 | aceQ | K25889 |
| pgaC | K11936 | amsE | K16702 | aceP | K25890 |
| pgaD | K11937 | wcaL | K16703 | aceR | K25891 |
| wecF | K12582 | amsG | K16707 | aceI | K25892 |
| gumD | K13656 | wcaK | K16710 | pssA | K25902 |
| gumH | K13657 | alg8 | K19290 | pssD | K25903 |
| gumI | K13658 | alg44 | K19291 | pssE | K25904 |
| gumK | K13659 | algX | K19293 | pssC | K25905 |
| gumM | K13660 | algI | K19294 | pssS | K25906 |
| gumF | K13663 | algJ | K19295 | pssF | K25907 |
| gumG | K13664 | algF | K19296 | pssI | K25908 |
| gumL | K13665 | vpsD | K20921 | pssG | K25909 |
| wcaE | K13683 | vpsI | K20922 | pssH | K25910 |
| wcaC | K13684 | pslA | K20997 | pssJ | K25911 |
| exoO | K16555 | pslF | K20999 | pssR | K25912 |
| exoM | K16556 | pslH | K21001 | pssK | K25913 |
| exoA | K16557 | pslI | K21002 | cpsG | K25957 |
| exoL | K16558 | pssM | K21154 | cpsF | K25958 |
| exoH | K16560 | | | | |

TABLE 2
Trehalose Biosynthesis Genes

| Gene | Gene ID |
|---|---|
| otsA | K00697 |
| otsB | K01087 |
| treM | K05343 |
| treP | K05342 |
| treT | K13057 |
| treY | K06044 |
| treZ | K01236 |

TABLE 3
Osmoprotectant Biosynthesis Genes

| Gene | Gene ID |
|---|---|
| betA | K00108 |
| ectA | K06718 |
| ectB | K00836 |
| ectC | K06720 |
| proA | K00147 |
| proB | K00931 |

TABLE 4
Pyruvate Metabolism Genes

| Gene | Gene ID |
|---|---|
| pps | K01007 |
| ppdK | K01006 |
| pyk | K00873 |
| pckG | K01596 |
| ppc | K01595 |
| pckA | K01610 |

TABLE 5
Pentose Phosphate Pathway Genes

| Gene | Gene ID |
|---|---|
| gdh | K00034, K18124, K18125, K22969 |
| gcd | K00117 |
| gnl | K01053 |
| gntK | K00851 |
| gnd | K00033 |
| rpiA | K01807 |
| prsA | K00948 |
| deoB | K01839 |

TABLE 6
Phosphonate and Phosphinate Metabolism Genes

| Gene | Gene ID | Gene | Gene ID |
|---|---|---|---|
| pepM | K01841 | phnM | K06162 |
| pphA | K19669 | phnJ | K06163 |
| ppd | K09459 | phnP | K06167 |
| phnX | K05306 | phnN | K05774 |
| fomC | K12901 | phnPP | K20859 |
| phpC | K12904 | phny | K00206 |
| mpnS | K18049 | phnA | K19670 |
| phnG | K06166 | phnW | K03430 |
| phnH | K06165 | phnO | K09994 |
| phnI | K06164 | pbfA | / |
| phnK | K05781 | phnY | K21195 |
| phnL | K05780 | phnZ | K21196 |

TABLE 7
Two-component system Genes

| Gene | Gene ID |
|---|---|
| phoU | K02039 |
| phoR | K07636 |
| phoB | K07657 |
| phoP | K07658, K07660 |
| SenX3 | K07768 |
| RegX3 | K07776 |
| pgtC | K08478 |
| pgtB | K08475 |
| pgtA | K08476 |

TABLE 8
Transporters Genes

| Gene | Gene ID | Gene | Gene ID |
|---|---|---|---|
| pgtP | K11382 | ugpB | K05813 |
| pstS | K02040 | ugpA | K05814 |
| pstC | K02037 | ugpE | K05815 |
| pstA | K02038 | ugpC | K05816 |
| pstB | K02036 | phnS | K11081 |
| pit | K03306 | phnV | K11082 |
| htxB | / | phnU | K11083 |
| ptxA | / | phnT | K11084 |
| ptxB | / | glpT | K02445 |
| ptxC | / | aepX | / |
| phnD_phosphite | / | aepV | / |
| phnD | K02044 | aepW | / |
| phnE | K02042 | aepP | / |
| phnC | K02041 | aepS | / |

TABLE 9
Organic phosphoester hydrolysis Genes

| Gene | Gene ID | Gene | Gene ID |
|---|---|---|---|
| opd | K07048 | phoC | / |
| pafA | / | olpA | K01078 |
| phoA | K01077 | phy | K01083 |
| phoD | K01113 | appA | K01093 |
| phoX | K07093 | ugpQ | K01126 |
| phoN | K09474 | glpQ | K01126 |
| aphA | K03788 | | |

TABLE 10
Purine Metabolism Genes

| Gene | Gene ID | Gene | Gene ID |
|---|---|---|---|
| purF | K00764 | purH | K00602 |
| purD | K01945 | purP | K06863 |
| purN | K11175 | purO | K11176 |
| purT | K08289 | guaB | K00088 |
| purL | K23269 | guaA | K01951 |
| purS | K23264 | gmk | K00942 |
| purQ | K23265 | ushA | K11751 |
| purM | K01933 | ndk | K00940 |
| purK | K01589 | spoT | K01139 |
| purE | K01588 | ppx | K01524 |
| ADE2 | K11808 | purA | K01939 |
| purC | K01923 | adk | K00939 |
| purB | K01756 | | |

TABLE 11
Pyrimidine Metabolism Genes

| Gene | Gene ID | Gene | Gene ID |
|---|---|---|---|
| pyrE | K00762 | nrdE | K00525 |
| pyrF | K01591 | nrdB | K00526 |
| ushA | K11751 | nrdF | K00526 |
| cmk | K00945 | nrdJ | K00524 |
| pyrH | K09903 | dcd | K01494 |
| ndk | K00940 | dut | K01520 |
| pyrG | K01937 | thyA | K00560 |
| rtpR | K00527 | tmk | K00943 |
| nrdD | K21636 | | |
| nrdA | K00525 | | |

TABLE 12
Nitrification Genes

| Gene | Gene ID |
|---|---|
| amoA_A | K10944 |
| amoB_A | K10945 |
| amoC_A | K10946 |
| amoA_B | K10944 |
| amoB_B | K10945 |
| amoC_B | K10946 |
| hao | K10535 |
| nxrA | K00370 |
| nxrB | K00371 |

TABLE 13
Denitrification Genes

| Gene | Gene ID |
|---|---|
| napA | K02567 |
| napB | K02568 |
| napC | K02569 |
| narG | K00370 |
| narH | K00371 |
| narJ | K00373 |
| narI | K00374 |

TABLE 13
Denitrification Genes

| Gene | Gene ID |
|---|---|
| nirK | K00368 |
| nirS | K15864 |
| norB | K04561 |
| norC | K02305 |
| nosZ | K00376 |
| narZ | K00370 |
| narY | K00371 |
| narV | K00374 |
| narW | K00373 |
| CYP55 | K15877 |

TABLE 14
Assimilatory Nitrate Reduction Genes

| Gene | Gene ID |
|---|---|
| nasA | K00372 |
| nasB | K00360, K00361 |
| nirA | K00366 |
| NR | K10534 |
| narB | K00367 |
| narC | K02569 |

TABLE 15
Dissimilatory Nitrate Reduction Genes

| Gene | Gene ID | Gene | Gene ID |
|---|---|---|---|
| napA | K02567 | narY | K00371 |
| napB | K02568 | narV | K00374 |
| napC | K02569 | narW | K00373 |
| narG | K00370 | nirB | K00362 |
| narH | K00371 | nirD | K00363 |
| narJ | K00373 | nrfA | K03385 |
| narI | K00374 | nrfB | K04013 |
| narZ | K00370 | nrfC | K04014 |
| | | nrfD | K04015 |

TABLE 16
Nitrogen Fixation Genes

| Gene | Gene ID |
|---|---|
| anfG | K00531 |
| nifD | K02586 |
| nifH | K02588 |
| nifK | K02591 |
| nifW | K02595 |

TABLE 17
Anammox Genes

| Gene | Gene ID |
|---|---|
| hzo | / |
| hzsA | K20932 |
| hzsB | K20933 |
| hzsC | K20934 |
| hdh | K20935 |

TABLE 18
Organic degradation and synthesis Genes

| Gene | Gene ID | Gene | Gene ID |
|---|---|---|---|
| ureA | K01430 | gdh_K15371 | / |
| ureB | K01429 | gs_K00264 | / |
| ureC | K01428 | gs_K00265 | / |
| nao | K19823 | gs_K00266 | / |
| nmo | K00459 | gs_K00284 | / |
| gdh_K00260 | / | glsA | K01425 |
| gdh_K00261 | / | glnA | K01915 |
| gdh_K00262 | / | asnB | K01953 |
| | | ansB | K01424 |

TABLE 19
Auxiliary Activities Genes

Gene
AA0
AA1
AA10
AA2
AA3
AA4
AA5
AA6
AA7
AA8
AA9

TABLE 20
Carbohydrate Binding Modules

| Gene | Gene | Gene | Gene | Gene | Gene |
|---|---|---|---|---|---|
| CBM0 | CBM2 | CBM30 | CBM41 | CBM52 | CBM63 |
| CBM1 | CBM20 | CBM31 | CBM42 | CBM53 | CBM64 |
| CBM10 | CBM21 | CBM32 | CBM43 | CBM54 | CBM65 |
| CBM11 | CBM22 | CBM33 | CBM44 | CBM55 | CBM66 |
| CBM12 | CBM23 | CBM34 | CBM45 | CBM56 | CBM67 |
| CBM13 | CBM24 | CBM35 | CBM46 | CBM57 | CBM7 |
| CBM14 | CBM25 | CBM36 | CBM47 | CBM58 | CBM8 |
| CBM15 | CBM26 | CBM37 | CBM48 | CBM59 | CBM9 |
| CBM16 | CBM27 | CBM38 | CBM49 | CBM6 | |
| CBM17 | CBM28 | CBM39 | CBM5 | CBM60 | |
| CBM18 | CBM29 | CBM4 | CBM50 | CBM61 | |
| CBM19 | CBM3 | CBM40 | CBM51 | CBM62 | |

TABLE 21
Carbohydrate Esterases

| Gene | Gene |
|---|---|
| CE0 | CE2 |
| CE1 | CE3 |
| CE10 | CE4 |
| CE11 | CE5 |
| CE12 | CE6 |
| CE13 | CE7 |
| CE14 | CE8 |
| CE15 | CE9 |
| CE16 | |

TABLE 22
Glycoside Hydrolases

| Gene | Gene | Gene | Gene | Gene | Gene |
|---|---|---|---|---|---|
| GH0 | GH118 | GH2 | GH4 | GH6 | GH8 |
| GH1 | GH119 | GH20 | GH40 | GH60 | GH80 |
| GH10 | GH12 | GH21 | GH41 | GH61 | GH81 |
| GH100 | GH120 | GH22 | GH42 | GH62 | GH82 |
| GH101 | GH121 | GH23 | GH43 | GH63 | GH83 |
| GH102 | GH122 | GH24 | GH44 | GH64 | GH84 |
| GH103 | GH123 | GH25 | GH45 | GH65 | GH85 |
| GH104 | GH124 | GH26 | GH46 | GH66 | GH86 |
| GH105 | GH125 | GH27 | GH47 | GH67 | GH87 |
| GH106 | GH126 | GH28 | GH48 | GH68 | GH88 |
| GH107 | GH127 | GH29 | GH49 | GH69 | GH89 |
| GH108 | GH128 | GH3 | GH5 | GH7 | GH9 |
| GH109 | GH129 | GH30 | GH50 | GH70 | GH90 |
| GH11 | GH13 | GH31 | GH51 | GH71 | GH91 |
| GH110 | GH130 | GH32 | GH52 | GH72 | GH92 |
| GH111 | GH131 | GH33 | GH53 | GH73 | GH93 |
| GH112 | GH132 | GH34 | GH54 | GH74 | GH94 |
| GH113 | GH14 | GH35 | GH55 | GH75 | GH95 |
| GH114 | GH15 | GH36 | GH56 | GH76 | GH96 |
| GH115 | GH16 | GH37 | GH57 | GH77 | GH97 |
| GH116 | GH17 | GH38 | GH58 | GH78 | GH98 |
| GH117 | GH18 | GH39 | GH59 | GH79 | GH99 |

TABLE 23
GlycosylTransferases
Gene

GT0
GT1
GT10
GT11
GT12
GT13
GT14
GT15
GT16
GT17
GT18
GT19
GT2
GT20
GT21
GT22
GT23
GT24
GT25
GT26
GT27
GT28
GT29
GT3
GT30
GT31
GT32
GT33
GT34
GT35
GT36
GT37
GT38
GT39
GT4
GT40
GT41
GT42
GT43
GT44
GT45
GT46
GT47
GT48
GT49
GT5
GT50
GT51
GT52
GT53
GT54
GT55
GT56
GT57
GT58
GT59
GT6
GT60
GT61
GT62
GT63
GT64
GT65
GT66
GT67
GT68
GT69
GT7
GT70
GT71
GT72
GT73
GT74
GT75
GT76
GT77
GT78
GT79
GT8
GT80
GT81
GT82
GT83
GT84
GT85
GT86
GT87
GT88
GT89
GT9
GT90
GT91
GT92
GT93
GT94

TABLE 24
Polysaccharide Lyase

| Gene | Gene | Gene |
|---|---|---|
| PL0 | PL16 | PL22 |
| PL1 | PL17 | PL3 |
| PL10 | PL18 | PL4 |
| PL11 | PL19 | PL5 |
| PL12 | PL2 | PL6 |
| PL13 | PL20 | PL7 |
| PL14 | PL21 | PL8 |
| PL15 | | PL9 |

TABLE 25
Immobilization Genes

| Gene | Gene ID |
|---|---|
| Nrt, narK, nrtP | K02575 |
| NR | K10534 |
| narT | K10850 |
| aspQ, ansB, ansA | K05597 |

TABLE 26
Mineralization Genes

| Gene | Gene ID |
|---|---|
| URE | K01427 |
| urtA | K11959 |
| urtB | K11960 |
| urtC | K11961 |
| urtD | K11962 |
| urtE | K11963 |
| E3.2.1.14 | K01183 |
| nagZ | K01207 |
| sacC | K01212 |
| csn | K01233 |
| E3.5.1.41 | K01452 |
| HEXA_B | K12373 |
| chiA | K13381 |
| HEX | K14459 |

TABLE 27
Sugars

| Gene | Gene ID | Gene | Gene ID |
|---|---|---|---|
| scrK | K00847 | treA, treF | K01194 |
| UGDH, ugd | K00012 | treC | K01226 |
| USP | K12447 | glk | K00845 |
| pgil | K06859 | rhaA | K01820 |
| pgm | K01835 | rhaB | K00848 |
| GPI, pgi | K01810 | rhaD | K01629 |
| KHK | K00846 | LRA1 | K18337 |
| fbp3 | K04041 | LRA2 | K18338 |
| ALDO | K01623 | LRA3, yfaW | K12661 |
| fbaB | K11645 | LRA4 | K18339 |
| FBA, fbaA | K01624 | rhaA | K01813 |
| bgaB | K12308 | FUK | K05305 |
| lacB | K01819 | fucI | K01818 |
| lacC | K00917 | fucK | K00879 |
| lacD | K01635 | fucA | K01628 |
| lacZ | K01190 | fucD | K18334 |
| galM | K01785 | araA | K01804 |
| galK | K00849 | araB | K00853 |
| galT | K00965 | araC | K13875 |
| galE | K01784 | ARD | K17738 |
| gal | K00035 | ARD1 | K17818 |
| | | dalD | K00007 |

TABLE 28
Polymers

| Gene | Gene ID | Gene | Gene ID |
|---|---|---|---|
| amyA, malS | K01176 | chbP | K18675 |
| E3.2.1.1A | K07405 | nagA | K01443 |
| E3.2.1.2 | K01177 | gspK | K18676 |
| E3.2.1.3 | K01178 | E3.1.1.11 | K01051 |
| E3.2.1.10 | K01182 | pel | K01728 |
| AGL | K01196 | pelW | K01731 |
| E3.2.1.91 | K01225 | pelC | K19551 |
| E3.2.1.4 | K01179 | ogl | K01730 |
| EGLD | K19356 | uxaC | K01812 |
| CELB | K19357 | ligX | K15060 |
| xynB | K01198 | ligZ | K15061 |
| xynD | K15921 | ligY | K15062 |
| xylB | K00854 | ligW | K15063 |
| xylA | K01805 | vanA | K03862 |
| XR | K17743 | vanB | K03863 |
| DCXR | K03331 | ligA | K04100 |
| lyxK | K00880 | ligB | K04101 |
| E1.1.1.9 | K05351 | ligC | K10219 |
| xdh | K14273 | ligI | K10221 |
| xad | K14275 | | |

TABLE 29
Aromatic

| Gene | Gene ID | Gene | Gene ID |
|---|---|---|---|
| badA | K04110 | E4.2.1.84 | K01721 |
| benA-xylX | K05549 | dmpH, xylI, nahK | K01617 |
| E1.14.13.12 | K07824 | tfdA | K06912 |
| lysA | K01586 | tfdB | K10676 |
| oadB | K01572 | dmpC, xylG | K10217 |
| gcdH | K00252 | E3.5.1.47 | K05823 |
| lysAC | K12526 | hyuA | K01473 |
| hdc | K01590 | bbsH | K07546 |
| benB-xylY | K05550 | catB | K01856 |
| hcaB | K05711 | E1.17.99.1 | K05797 |
| HGD, hmgA | K00451 | xylC | K00141 |
| fahA | K01555 | desB, galA | K04099 |
| maiA | K01800 | bphA | K08689 |
| mdlA | K01781 | deoC, DERA | K01619 |
| mdlB | K15054 | E1.2.1.10 | K00132 |
| mdlC | K01576 | pcaG | K00448 |
| mhpA | K05712 | pcaH | K00449 |
| E1.13.11.4 | K00450 | E1.14.13.1 | K00480 |
| FAHD1 | K01557 | CYP81F | K00517 |
| hcaF, hcaA2 | K05709 | E1.14.12.7 | K07519 |
| hcaE, hcaA1 | K05708 | nidA | K11943 |
| pht5 | K04102 | nidB | K11944 |
| phbB | K00023 | phdF | K11945 |
| pobA | K00481 | phdG | K11946 |
| amiD | K11066 | nidD | K11947 |
| amiE | K01426 | phdI | K11948 |
| E1.1.1.90 | K00055 | phdJ | K11949 |
| catA | K03381 | nahAb, nagAb, ndoA | K14578 |
| dmpB, xylE | K00446 | nahAc,ndoB | K14579 |
| E3.5.5.1 | K01501 | nahAd, ndoC | K14580 |
| E1.14.13.7 | K03380 | nahAa, nagAa, ndoR | K14581 |
| flnD2 | K14603 | nahB, doxE | K14582 |
| E1.14.13.22 | K03379 | dbfA1 | K14599 |
| E1.2.1.3 | K00128 | dbfA2 | K14600 |
| ALDH9A1 | K00149 | flnB | K14601 |
| chqB | K04098 | flnD1 | K14602 |
| HAAO | K00452 | | |

TABLE 30
Sulfur Metabolism

| Gene | Gene ID |
|---|---|
| cysN | K00956 |
| cysD | K00957 |
| sat, met3 | K00958 |
| aprA | K00394 |
| aprB | K00395 |
| dsrA | K11180 |
| dsrB | K11181 |

TABLE 31
Methanogen

| Gene | Gene ID | Gene | Gene ID |
|---|---|---|---|
| ackA | K00925 | mtrF | K00582 |
| E2.3.1.8 | K00625 | mtrG | K00583 |
| pta | K13788 | mtrH | K00584 |
| ACSS, acs | K01895 | mcrA | K00399 |
| cdhC | K00193 | mcrB | K00401 |
| cdhE, acsC | K00197 | mcrG | K00402 |
| cdhD, acsD | K00194 | hdrA | K03388 |
| mtaA | K14080 | hdrB | K03389 |
| mtaB | K04480 | hdrC | K03390 |
| mtaC | K14081 | fwdA, fmdA | K00200 |
| mtbA | K14082 | fwdB, fmdB | K00201 |
| mtmC | K16177 | fwdC, fmdC | K00202 |
| mtmB | K16176 | fwdD, fmdD | K00203 |
| mtbC | K16179 | fwdE, fmdE | K11261 |
| mtbB | K16178 | fwdF, fmdF | K00205 |
| mttC | K14084 | fwdG | K11260 |
| mttB | K14083 | fwdH | K00204 |
| mtrA | K00577 | ftr | K00672 |
| mtrB | K00578 | mch | K01499 |
| mtrC | K00579 | mtd | K00319 |
| mtrD | K00580 | hmd | K13942 |
| mtrE | K00581 | mer | K00320 |

TABLE 32
Additional Genes

| | Gene | | |
|---|---|---|---|
| Gene | ID | Class | Additional Details |
| nrfH | K15876 | Ammonification | / |
| narB | K15878 | Ambig_N03r | rieske iron-sulfur protein |
| narC | K15879 | Ambig_N03r | cytochrome b-561 |
| accD | K01505 | ethylene breakdown | / |
| iaaM | K00466 | Auxin biosynthesis | / |
| iaaH | K21801 | Auxin biosynthesis | / |
| ptsI | K08483 | Phosphotransferase | / |
| | | system | |
| ptsH | K02784, | Phosphotransferase | / |
| | K11189 | system | |
| ppk | K00937 | Oxidative | / |
| | | phosphorylation | |
| ppa | K01507 | Oxidative | |
| | | phosphorylation | |
| ppaC | K15986 | P_scavenging | manganese-dependent |
| | | | inorganic |
| | | | pyrophosphatase |
| PRUNE, | K01514 | P_scavenging | exopolyphosphatase |
| PPX1 | | | |
| htxA | / | Others | hypophosphite |
| | | | dioxygenase |
| ptxD | K18916 | Others | phosphonate |
| | | | dehydrogenase |
| lysR | / | Others | diaminopimelate |
| | | | decarboxylase |
| phnR | / | Others | transcriptional |
| | | | regulator of 2- |
| | | | aminoethylphosphonate |
| | | | degradation operons |
| phnF | K02043 | Others | GntR family transcriptional |
| | | | regulator, phosphonate |
| | | | transport system regulatory |
| | | | protein |
| phoH | K06217 | Others | phosphate starvation- |
| | | | inducible protein |
| hcp | K05601 | others | hydroxylamine reductase |
| pmoA | K10944 | others | methane/ammonia |
| | | | monooxygenase subunit A |
| pmoB | K10945 | others | methane/ammonia |
| | | | monooxygenase subunit B |
| pmoC | K10946 | others | methane/ammonia |
| | | | monooxygenase subunit C |
| cohesin | / | Cellulosome | / |
| dockerin | / | Cellulosome | / |
| SLH | / | Cellulosome | / |

### Beneficial Microorganism

In some embodiments, based on the analysis of sample disclosed herein, a beneficial microorganism is detected. In some embodiments, based on the analysis of sample disclosed herein, a beneficial microorganism is recommended for treatment of the soil or environment from which the sample was collected. In some embodiments, based on the analysis of sample disclosed herein, a beneficial microorganism is detected following treatment of the soil or environment from which the sample was collected. In some embodiments, following a treatment disclosed herein, one or more beneficial microorganisms is increased, for example one or more of Arthrobacter, Aspergillus, Azospirillum, Azotobacter, Bacillus, Burkholderia, Enterobacter, Klebsiella, Lysinibacillus, Paenibacillus, Pseudomonas, Penicillium, Serratia, Lacticaseibacillus, Lactiplantibacillus and/or Streptomyces. Beneficial microorganisms may include but are not limited to Mycorrhizal Fungi, Rhizobium bacteria, Azotobacter bacteria, Azospirillum bacteria, Bacillus spp., Pseudomonas spp., Trichoderma spp, Actinomycetes, Lactic Acid Bacteria (LAB), Streptomyces spp., Entomopathogenic fungi, Rhizobium, Azospirillum, Azotobacter, Frankia, Mycorrhizal fungi (e.g., Glomus spp.), Trichoderma, Bacillus subtilis, Bacillus thuringiensis, Pseudomonas fluorescens, Pseudomonas putida, Streptomyces, Saccharomyces cerevisiae (brewer's yeast), Lactobacillus, Clostridium, Agrobacterium, Enterobacter, Nitrosomonas, Nitrobacter, Actinomycetes, Penicillium, Aspergillus, Lysobacter, Arbuscular mycorrhizal fungi (AMF), Burkholderia, Bradyrhizobium, Clavibacter, Methylobacterium, Comamonas, Rhodospirillum, Streptomyces griseus, Streptomyces avermitilis, Streptomyces coelicolor, Streptomyces hygroscopicus, Streptomyces venezuelae, Streptomyces antibioticus, Streptomyces albus, Streptomyces noursei, Streptomyces aureofaciens, Streptomyces roseosporus, Streptomyces lividans, Streptomyces parvulus, Streptomyces chattanoogensis, Streptomyces griseoluteus, Streptomyces nogalater, Streptomyces fradiae, Streptomyces erythraeus, Streptomyces tanashiensis, Streptomyces kanamyceticus, Streptomyces lydicus, Bacillus licheniformis, Bacillus amyloliquefaciens, Bacillus megaterium, Bacillus coagulans, Bacillus pumilus, Bacillus cereus, Bacillus polymyxa, Bacillus circulans, Bacillus firmus, Bacillus popilliae, Bacillus pasteurii, Bacillus macerans, Bacillus mucilaginosus, Bacillus azotofixans, Bacillus brevis, Bacillus stearothermophilus, Bacillus sphaericus, Bacillus acidocaldarius, Bacillus halodurans, Bacillus smithii, Bacillus marinus, Bacillus infemus, Bacillus alcalophilus, Bacillus psychrophilus, Bacillus halodenitrificans, Bacillus thermoglucosidasius, Bacillus thermoleovorans, Bacillus boroniphilus, Bacillus schlegelii, Bacillus proteolyticus, Bacillus thermocloacae, Bacillus thermoamylovorans, Bacillus amylolyticus, Bacillus lautus, Bacillus cohnii, Bacillus phycophilus, Bacillus chroococcidiopsidis, Bacillus methanolicus, Bacillus psychrotolerans, Bacillus azotoformans, Bacillus fluvialis, Bacillus lautus, Bacillus conterminus, Bacillus fusiformis, Bacillus korlensis, Bacillus tropicus, Bacillus arsenicoselenatis, Bacillus siralis, Bacillus ehimensis, Bacillus toyonensis, Bacillus nakamurai, and any combination thereof.

In some embodiments, a beneficial microorganism is one that confers a benefit to a plant. In some embodiments, beneficial microorganisms can be present in the rhizosphere. In some embodiments, beneficial microorganisms can be obtained in a sample of the rhizosphere. In some embodiments, beneficial microorganisms can be introduced into the rhizosphere from an outside source. In some embodiments, beneficial microorganisms can be introduced into the rhizosphere using a plant management technique. In some embodiments, a plant management technique may be selected using a score calculated as described herein.

In some embodiments, the benefit conferred by a beneficial microorganism may comprise but is not limited to resistance to an environmental stressor, resistance to a disease, resistance to a pest, resistance to a pesticide, reduced susceptibility to a disease, reduced susceptibility to a pest, reduced susceptibility to a pesticide, or improved nutrient uptake. In some embodiments, the environmental stressors may comprise drought, flood, salinity, heat, cold, ozone, UV radiation, heavy metals, or nutrient deficiency, among others. In some embodiments, a disease may comprise stress, root rot, damping-off, vascular wilt, nutritional deficiency, salt injury, or infection caused by fungi, oomycetes, bacteria, viruses, viroids, virus-like organisms, phytoplasmas, protozoa, nematodes, or parasitic plants. In some embodiments, a pest comprises an aphid, a thrip, a mite, a leaf miner, a fly, an earwig, a gnat, a mealybug, a worm, a beetle, a caterpillar, a cicada, a slug, a snail, a moth, a cricket, an ant, or a nematode. In some embodiments, a pesticide comprises one or more of an organochlorine, an organophosphate, an organosulfur, a carbamate, a formamidine, a dinitrophenol, an organotin, a pyrethroid, a nicotinoid, a spinosyn, a pyrazole, a pyridazinone, a quinazoline, a botanical, a synergist, an antibiotic, a fumigant, an inorganic, a biorational, a benzoylurea, an herbicide, an insecticide, or a fungicide. In some embodiments, a nutrient comprises nutrient comprises nitrogen, phosphorus, potassium, calcium, magnesium, sulfur, iron, zinc, manganese, copper, boron, molybdenum, or chlorine.

In some embodiments, a functional attribute possessed by a beneficial microorganism of the present disclosure includes, but is not limited to the ability to impart one or more beneficial properties to a plant species, for example, increased growth, increased yield, increased nitrogen utilization efficiency, increased stress tolerance, increased drought tolerance, increased photosynthetic rate, enhanced water use efficiency, increased pathogen resistance, or modifications to plant architecture that do not necessarily impact plant yield, but rather address plant functionality.

In some embodiments, the ability to impart the disclosed beneficial properties upon a plant is not possessed by an individual microorganisms. In some embodiments, the ability to impart the disclosed beneficial properties upon a plant is not possessed by individual microorganisms as they would occur in nature. In some embodiments, it is by human intervention applying these microorganisms to a plant that a functional composition is developed, said functional composition possessing attributes and functional properties that do not exist in nature.

In some embodiments, beneficial microorganisms of the present disclosure can be used to promote plant growth, to function as growth modifiers, to enhance soil health, to enhance plant health, to improve grain, fruit, or flower yield, to improve crop yield, to improve disease resistance, to improve stress resistance, to improve the growth of plant parts, to improve or encourage the development of beneficial plant phenotypes, or any combination thereof. In some embodiments, beneficial microorganisms of the present disclosure may comprise but are not limited to one of more of the following genera: Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, and/or Streptomyces. Beneficial microorganisms of the present disclosure may include but are not limited to Mycorrhizal Fungi, Rhizobium bacteria, Azotobacter bacteria, Azospirillum bacteria, Bacillus spp., Pseudomonas spp., Trichoderma spp, Actinomycetes, Lactic Acid Bacteria (LAB), Streptomyces spp., Entomopathogenic fungi, Rhizobium, Azospirillum, Azotobacter, Frankia, Mycorrhizal fungi (e.g., Glomus spp.), Trichoderma, Bacillus subtilis, Bacillus thuringiensis, Pseudomonas fluorescens, Pseudomonas putida, Streptomyces, Saccharomyces cerevisiae (brewer's yeast), Lactobacillus, Clostridium, Agrobacterium, Enterobacter, Nitrosomonas, Nitrobacter, Actinomycetes, Penicillium, Aspergillus, Lysobacter, Arbuscular mycorrhizal fungi (AMF), Burkholderia, Bradyrhizobium, Clavibacter, Methylobacterium, Comamonas, Rhodospirillum, Streptomyces griseus, Streptomyces avermitilis, Streptomyces coelicolor, Streptomyces hygroscopicus, Streptomyces venezuelae, Streptomyces antibioticus, Streptomyces albus, Streptomyces noursei, Streptomyces aureofaciens, Streptomyces roseosporus, Streptomyces lividans, Streptomyces parvulus, Streptomyces chattanoogensis, Streptomyces griseoluteus, Streptomyces nogalater, Streptomyces fradiae, Streptomyces erythraeus, Streptomyces tanashiensis, Streptomyces kanamyceticus, Streptomyces lydicus, Bacillus licheniformis, Bacillus amyloliquefaciens, Bacillus megaterium, Bacillus coagulans, Bacillus pumilus, Bacillus cereus, Bacillus polymyxa, Bacillus circulans, Bacillus firmus, Bacillus popilliae, Bacillus pasteurii, Bacillus macerans, Bacillus mucilaginosus, Bacillus azotofixans, Bacillus brevis, Bacillus stearothermophilus, Bacillus sphaericus, Bacillus acidocaldarius, Bacillus halodurans, Bacillus smithii, Bacillus marinus, Bacillus infemus, Bacillus alcalophilus, Bacillus psychrophilus, Bacillus halodenitrificans, Bacillus thermoglucosidasius, Bacillus thermoleovorans, Bacillus boroniphilus, Bacillus schlegelii, Bacillus proteolyticus, Bacillus thermocloacae, Bacillus thermoamylovorans, Bacillus amylolyticus, Bacillus lautus, Bacillus cohnii, Bacillus phycophilus, Bacillus chroococcidiopsidis, Bacillus methanolicus, Bacillus psychrotolerans, Bacillus azotoformans, Bacillus fluvialis, Bacillus lautus, Bacillus conterminus, Bacillus fusiformis, Bacillus korlensis, Bacillus tropicus, Bacillus arsenicoselenatis, Bacillus siralis, Bacillus ehimensis, Bacillus toyonensis, Bacillus nakamurai, and any combination thereof.

In some embodiments, beneficial microorganisms are applied to a plant or an environment to enhance a plant quality. In some embodiments, the plant quality is one or more of nitrogen fixation, stress tolerance, nutrient cycling, nutrient utilization, growth rate, disease resistance, grain or crop yield, or health. Nutrients may comprise but are not limited to one or more of nitrogen, oxygen, phosphorous, calcium, iron, sodium, potassium, sulfur, magnesium, chlorine, hydrogen, iron, boron, manganese, zinc, copper, nickel, and molybdenum. Cycling of nutrients may comprise but is not limited to one or more of the following processes: sorption, desorption, precipitation, dissolution, plant uptake, tile flow, sedimentation, resuspension, erosion, runoff, leaching, mineralization, immobilization, fixation, assimilation, evaporation, respiration, recycling, lysis, transpiration, combustion, and deposition.

In some embodiments, plant growth promoting rhizobacteria is added to the soil or environment from which the sample was collected following analysis of a sample disclosed herein. In some embodiments, beneficial microorganisms are added to the soil or environment from which the sample was collected following analysis of a sample disclosed herein to promote plant growth. In some embodiments, plant growth promotion can comprise (a) fixation of atmospheric nitrogen so that it can be used by the plant, (b) increase solubilization of minerals or the synthesis of phytohormones.

### Plant Management

In some embodiments, biostimulants are applied to a plant or an environment to enhance a plant quality. In some embodiments, the plant quality is one or more of nutrient uptake, development of soil microorganisms, or stimulating root growth to increase water use efficiency.

In some embodiments, a plant biostimulant may comprise a substance or microorganism applied to a plant. In some embodiments, the plant biostimulant may enhance nutrition efficiency, stress tolerance, or crop quality traits. In some embodiments, the plant biostimulants may comprise commercial products. In some embodiments, the plant biostimulants may comprise mixtures of such substances and/or microorganisms. In some embodiments, the biostimulants may improve nutrition of plants, crops, and/or soil. In some embodiments, the biostimulants themselves may not necessarily offer nutritional content. In some embodiments, a biostimulant can comprise enzymes, proteins, amino acids or micronutrients. In some embodiments, a biostimulant can be a natural biostimulant. In some embodiments, a biostimulant can comprise phenols, salicylic acid, humic, fulvic acids, or protein hydrolases. In some embodiments, biofertilizers can be a subcategory of biostimulants. In some embodiments, the biofertilizers can contain microbes. In some embodiments, the microbes can include, but are not limited to bacteria, archaebacteria, actinomycetes, fungi, algae, protozoans, viruses, viroids, nematodes, or any combination or portion thereof. In some embodiments, the microbes used in biofertilizers can be categorized as Plant Growth-Promoting Bacteria (PGPB). In some embodiments, the microbes used in biofertilizers can be categorized as Plant Growth-Promoting Rhizobacteria (PGPR).

In some embodiments, a fertilizer may comprise microbes. In some embodiments, the microbes can comprise but are not limited to one or more of the following genera: Arthrobacter, Aspergillus, Azospirillum, Azotobacter, Bacillus, Burkholderia, Enterobacter, Klebsiella, Lacticaseibacillus, Lactiplantibacillus, Lysinibacillus, Paenibacillus, Pseudomonas, Penicillium, Serratia, and/or Streptomyces.

In some embodiments, a plant can display phenotypic improvement following treatment (plant/soil/environment) with a biostimulant. In some embodiments, the phenotype improvement is disease resistance, heat tolerance, cold tolerance, salinity tolerance, metal tolerance, herbicide tolerance, chemical tolerance, improved nitrogen utilization, improved resistance to nitrogen stress, improved nitrogen fixation, pest resistance, herbivore resistance, pathogen resistance, increased yield, health enhancement, vigor improvement, growth improvement, photosynthetic capability improvement, nutrition enhancement, altered protein content, altered oil content, increased biomass, increased shoot length, increased root length, improved root architecture, increased seed weight, altered seed carbohydrate composition, altered seed oil composition, number of pods, delayed senescence, stay-green, and altered seed protein composition, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, or increased number of non-wilted leaves per plant.

In some embodiments, the methods described herein modulate a trait of agronomic importance. In some embodiments, the trait of agronomic importance can be, e.g., disease resistance, drought tolerance, heat tolerance, cold tolerance, salinity tolerance, metal tolerance, herbicide tolerance, chemical tolerance, improved water use efficiency, improved nitrogen utilization, improved resistance to nitrogen stress, improved nitrogen fixation, pest resistance, herbivore resistance, pathogen resistance, increased yield, increased yield under water-limited conditions, health enhancement, vigor improvement, growth improvement, photosynthetic capability improvement, nutrition enhancement, altered protein content, altered oil content, increased biomass, increased shoot length, increased root length, improved root architecture, increased seed weight, altered seed carbohydrate composition, altered seed oil composition, number of pods, delayed senescence, stay-green, or altered seed protein composition.

In some embodiments, the plants described herein are provided a benefit following treatment (plant/soil/environment) that is, for example, increase root biomass, increase root length, increase height, increase shoot length, increase leaf number, increase water use efficiency, increase overall biomass, increase grain yield, increase photosynthesis rate, increase tolerance to drought, increase heat tolerance, increased salt tolerance, increase resistance to nematode stress, increase resistance to a fungal pathogen, increase resistance to a bacterial pathogen, increase resistance to a viral pathogen, a detectable modulation in the level of a metabolite, or a detectable modulation in the proteome relative to a reference plant. In some embodiments, at least two benefits are provided to the plant following treatment.

In some embodiments, of the plants described herein, the population is treated in an amount effective to increase resistance to one or more of drought stress, heat stress, cold stress, salt stress, and low mineral stress. For example, the population can be treated in an amount effective to increase resistance to any biotic stress condition selected from a nematode stress, insect herbivory stress, fungal pathogen stress, bacterial pathogen stress, or viral pathogen stress.

In some embodiments, the present disclosure comprises use of plant management techniques. In some embodiments, a plant management technique can comprise the application of a system, method, or composition to provide benefit to a plant. In some embodiments, a plant management technique can comprise but is not limited to the application of a biofertilizer, a biostimulant, a beneficial microorganism, a fertilizer, a pesticide, a stimulant, or a modifier of plant qualities to a plant, soil or environment. In some embodiments, analysis of a sample disclosed herein detects the need for a specific plant management technique to increase a plant attribute or quality. In some embodiments, a plant quality that can be modified includes but is not limited to oil content, protein content, seed carbohydrate composition, seed oil composition, seed protein composition, chemical tolerance, cold tolerance, delayed senescence, disease resistance, drought tolerance, weight, growth improvement, health enhancement, heat tolerance, herbicide tolerance, herbivore resistance, nitrogen fixation, nitrogen utilization, root architecture, water use efficiency, biomass, root length, seed weight, shoot length, yield, yield under water-limited conditions, kernel mass, kernel moisture content, metal tolerance, number of ears, number of kernels per ear, number of pods, nutrition enhancement, pathogen resistance, pest resistance, photosynthetic capability, salinity tolerance, stay-green, vigor, dry weight of mature seeds, fresh weight of mature seeds, number of mature seeds per plant, chlorophyll content, number of pods per plant, length of pods per plant, number of wilted leaves per plant, number of severely wilted leaves per plant, number of non-wilted leaves per plant, a detectable modulation in the level of a metabolite, a detectable modulation in the level of a transcript, or a detectable modulation in the proteome relative to a reference plant.

In some embodiments, samples may be collected from bulk soil for comparison with rhizosphere soil. The bulk soil may be collected from a field. The bulk soil may be analyzed for comparison with the rhizosphere. The comparison may describe metagenomic changes in the rhizosphere from bulk or in-field samples.

In some embodiments, implementing a plant management technique may have a beneficial effect on or provide a benefit to a plant or crop. An effect of a plant management technique may comprise but is not limited to an improve phenotype of agronomic interest comprising disease resistance, drought tolerance, heat tolerance, cold tolerance, salinity tolerance, metal tolerance, herbicide tolerance, chemical tolerance, improve water use efficiency, improve nitrogen utilization, improve nitrogen fixation, pest resistance, herbivore resistance, pathogen resistance, increase yield, increase yield under water-limited conditions, health enhancement, vigor improvement, growth improvement, photosynthetic capability improvement, nutrition enhancement, altered protein content, altered oil content, increase biomass, increase shoot length, increase root length, improve root architecture, increase seed weight, altered seed carbohydrate composition, altered seed oil composition, number of pods, delayed senescence, stay-green, or altered seed protein composition.

In some embodiments, a plant management technique may comprise a soil treatment. In some embodiments, the soil treatment may comprise one or more of Trichoderma harzianum, Bacillus amyloliquefaciens, Bacillus subtilis, Myxococcus xanthus, Wickerhamomyces anomalus, Azotobacter vinelandii, Frateuria aurantia, Pseudomonas chlororaphis, Starmerella bombicola, Saccharomyces boulardii, Pichia occidentalis, Pichia kudriavzevii, or Meyerozyma guilliermondii.

In some embodiments, a plant management technique can be implemented after the analysis of a rhizosphere sample. In some embodiments, a plant management technique can be implemented after the analysis of a soil, plant, water, air, or germination paper sample. In some embodiments, a plant management technique can be implemented before the analysis of a rhizosphere sample. In some embodiments, a plant management technique can be implemented before the analysis of a soil, plant, water, air, or germination paper sample. In some embodiments, a plant management technique can be implemented after the collection of a sample. In some embodiments, a plant management technique can be implemented before the collection and/or analysis of a sample. In some embodiments, a plant management technique can be implemented one or more times. In some embodiments, a plant management technique can be implemented as needed following repeated analysis of a plant or location. In some embodiments, a plant management technique can be implemented in a field, plot, greenhouse, planter, hydroponic setup, or other environment in which a plant is growing or can be grown at some time in the future. In some embodiments, a plant management technique can be implemented once or more than once. In some embodiments, a plant management technique can be implemented multiple times over a period of time. Factors that can be considered in the determination to implement a plant management technique include but are not limited to: plant or crop variety, growth or health requirements of a particular plant or crop, soil composition, nutrient composition, climate, weather patterns, season, sun or shade cover, commercial factors such as crop production demands, water availability, detection of pests or diseases, or pertinent environmental factors related to farming. A plant management technique may comprise but is not limited to the application of one or more of a biofertilizer, a biostimulant, a modification in the amount of water supplied to a plant, a nutrient, a soil enrichment technique, a beneficial microorganism, a pesticide, an herbicide, a change in temperature of the plant environment, a change in humidity of the plant environment, a change in the availability and/or composition of the light in the plant environment, a change in the air handling of the plant environment, or any combination thereof.

### Attractants

In an aspect of the present disclosure, an attractant can be used to generate a rhizosphere. In some embodiments, an attractant can be placed in soil or an environment for growing a plant to analyze the soil or said environment. In some embodiments, a device can comprise the attractant. In some embodiments, an attractant can comprise a plant component or a microbe. In some embodiments, an attractant can comprise a seed. In some embodiments, a seed or plant can be a dicot, e.g., soybean, canola, cotton, tomato or pepper. In some embodiments, a seed or plant can be a monocot, e.g., corn, wheat, barley and rice. In some embodiments, a seed or plant can be transgenic. In some embodiments, a seed or plant can be genetically modified organism (GMO). In some embodiments, a plant can be a crop plant, a non-crop plant, a cultivated plant, or a non-cultivated plant. In some embodiments, the plant component and/or microbe may release exudates into the environment. In some embodiments, the exudates can attract a microbe. In some embodiments, an attractant can release exudates.

In some embodiments, the plant exudates include but are not limited to seed exudates and/or root exudates. In some embodiments, plant exudates can include secretions, diffusates, and lysates released from said plant into an environment surrounding said plant. In some embodiments, the environment can comprise one or more of soil, water, air, or germination paper. In some embodiments, lysates can be released from a plant by cell disruption of the cortical. In some embodiments, lysates can be released from a plant by cell disruption of the epidermal region. In some embodiments, the chemical nature of exudates from plants can act as an attractant in rhizospheres. In some embodiments, microorganisms can migrate toward or grow toward an attractant. In some embodiments, the metabolite profile of compounds exuded into a rhizosphere by a plant or attractant can be shaped by different internal and external factors. Non-limiting examples of internal factors can include but are not limited to species type, growth form, and functional characteristics of plants. In some embodiments, the internal factors can influence and/or modify the exudate composition in the rhizosphere. In some embodiments, rhizospheric microbial communities, herbivores, and/or plants growing nearby can also define the metabolite composition of a rhizosphere ecosystem as exogenous biotic factors. In some embodiments, the diverse group of chemical compounds released from plants can comprise but are not limited to sugars, fatty acids, amino acids, small peptides, polypeptides, organic acids, growth factors, enzymes, nucleotides, hormones, vitamins, phenolics, volatiles, or stimulants.

In some embodiments, plant exudates can affect microbial growth in the rhizosphere. In some embodiments, the concentration of microorganism cells can be several folds higher in the rhizosphere than in background soil lacking plants. In some embodiments, communities of microorganisms within the rhizosphere can be diverse, active, and/or behaving in synergy within a given community and/or between communities within a rhizosphere. In some embodiments, plant exudates, including but not limited to seed exudates and root exudates, can influence the growth of bacteria and/or fungi that colonize a rhizosphere. In some embodiments, plant exudates can serve as growth substrates for microorganisms.

In some embodiments, one or more attractant disclosed herein is placed in soil or an environment for growing a plant. In some embodiments, a sample from the soil or environment for growing the plant is analyzed as described herein after placement of the attractant to determine soil/environmental health, plants that would grow well therein, plants that would not grow well therein, treatments for administration. In some embodiments, the sample is collected at least 1, 5, 10, 15, 20, 30, 45, 60, 70, 100 days following placement of the attractant. In some embodiments, a plant management technique described herein is implemented following analysis.

### Sequencing Methods

An aspect of the present disclosure is the sequencing of genetic material obtained from a rhizosphere. The term “sequencing” as used herein can refer to sequencing methods for determining the order of the nucleotide bases—adenine, guanine, cytosine, uracil, and thymine—in a nucleic acid molecule (e.g., a DNA or RNA nucleic acid molecule).

For example, in ascertaining microbiome information the selection and sequencing of particular regions or portions of genetic materials can be used, including for example, the SSU rRNA gene (16S or 18S), the LSU rRNA gene (23S or 28S), the ITS in the rRNA operon, cpn60, gene marker regions such as metal-dependent proteases with possible chaperone activity, and/or various other segments consisting of base pairs, peptides and/or polysaccharides for use in characterizing the microbial community and the relationships among its constituents.

In some embodiments, a method of the present disclosure can comprise one or more of the following, which can be conducted in various orders and certain procedures can be omitted: sample preparation including obtaining the sample at the designated location, and manipulating the sample; extraction of the genetic material and other biomolecules from the microbial communities in the sample; preparation of libraries with identifiers such as an appropriate barcode such as DNA libraries, metabolite libraries, and protein libraries of the material; sequence elucidation of the material (including, for example, DNA, RNA, and protein) of the microbial communities in the sample; processing and analysis of the sequencing and potentially other molecular data; and exploitation of the information.

In some embodiments, sampling can be from an agricultural location, plants, soil, surfaces, or liquids. The samples can include for example solid samples such as soil, sediment, rock, or food. In some embodiments, genetic material is obtained from the sample.

In some embodiments, accuracy of analyses disclosed herein can depend on the choice of primers. In some embodiments, primers can be prepared by a variety of methods including, but not limited to, cloning of appropriate sequences and direct chemical synthesis. In some embodiments, primers can also be obtained from commercial sources such as Integrated DNA Technologies, Operon Technologies, Amersham Pharmacia Biotech, Sigma, and Life Technologies. In some embodiments, computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering.

In some embodiments, a sequencing pipeline is used to sequence and analyze nucleic acids obtained by the methods disclosed herein. The sequencing pipeline may comprise programs including but not limited to Kraken2, Metagenome-QC, FragGeneScan, Diamond, MicrobeCensus, SourMash, and any combination thereof. A PDF report may be generated comprising data analyzed using the programs listed herein. The PDF report may be generated using Metrics generation and/or Build Report PDF. Cloud infrastructure may be used to carry out data analysis, data storage, or any other analytical or data-related process disclosed herein. The bioinformatic pipeline may be used to process shotgun sequencing files obtained using the methods disclosed herein. The bioinformatic pipeline may identify bacteria, fungi, or other microorganisms which comprise the rhizomicrobiome or soil sample community. The bioinformatic pipeline may be triggered when a shotgun sequencing file is uploaded to an azure container. The bioinformatic pipeline may be event driven. The bioinformatic pipeline may upload an output file. Programs such as Metagenome-Atlas QC may be used for quality control of sequencing data. Quality control of sequencing data may comprise PCR duplicates removal, quality trimming, host removal, common contaminants removal, or any combination thereof. Programs such as FragGeneScan may be used to predict genes found in short or error-prone nucleic acid sequences. Programs such as FragGeneScan may be applied upstream of DIAMOND to identify and extract coding regions to obtain a higher fidelity account of genes present. Programs such as DIAMOND may be used to align sequences such as protein sequences against reference databases. Alignment of sequences may be used to quantify functional profiles. Programs such as Kraken2 may be used to apply taxonomic labels to metagenomic nucleic acid sequences by comparing them to pre-built libraries. Programs such as MicrobeCensus may be used to approximate average genome size as a means of quantifying disturbance rating. Programs such as SourMash may be used to perform high fidelity analysis of genomic sequences against pathogenic reference libraries to detect presence of pathogens.

In some embodiments, the bioinformatic pipeline disclosed herein may process and analyze metagenomic nucleic acid samples using untargeted taxonomic and functional methods. Output reports generated summarizing data obtained herein may be compared against a reference population of soil samples. Programs such as BBtools may be used to carry out several quality control steps. Quality control steps may include but are not limited to the removal of common adapter sequences and known contaminants, the removal of human reads, the conversion of .fastq to .fasta format, and the quantification of ectomycorrhizal and arbuscular mycorrhizal fungi. Programs such as FragGeneScan may be used to predict open reading frames on short DNA reads. Predicting open reading frames on short DNA reads may be used to convert DNA sequences to protein sequences. Converting DNA sequences to protein sequences may be used to search against protein databases without six frame translation. Programs such as Kofamscan may be used to search short amino acid sequences against KO HMM profiles to generate best hit KO for each sequence. Once a best hit KO is established for each sequence, custom code can be used to generate reads per kilobase million values for each KO in the sample by dividing KO counts by KO length in kilobases and number of million reads. Programs such as DIAMOND may be used to search short amino acid sequences against the COG protein database to generate best hit COG for each sequence. Once a best hit COG for each sequence is established, custom code can be used to generate reads per kilobase million values for each COG in the sample by dividing COG counts by COG length in kilobases and number of million reads. Programs such as Kraken2/Bracken may be used to assign taxonomy to reads against the PlusPF database. Bracken may be used to estimate percent abundance of taxa in samples. Programs such as SourMash may be used to search for specific bacterial and fungal pathogens in a sample. This search may be done using kmer and FracMinHash methods in which reads and targeted pathogens are each separately decomposed into kmers and then containment between kmer sets is identified by FracMinHash. SourMash may be used to generate a kmer signature for the reads which is then searched in the report building process. Kmer profiles may be built for bacterial pathogen searches and fungal pathogen searches. The analytical processes described herein may be applied to soil samples, rhizosphere samples, manure samples, or any combination thereof. Various factors may be quantified including number of genera, bacteria to fungal ratio, ectomycorrhizal to arbuscular ratio, mycorrhizae abundance, Shannon's diversity, Pielou Evenness, Functions of Interest (such as, for example, anoxic environment, lox oxygen environment, carbon fixation, organic carbon breakdown, methanogenesis, denitrification, nitrification, nitrogen fixation, organic nitrogen breakdown, phosphorous mobilization, potassium solubilization, sulfur oxidation, sulfur reduction, calcium transport, iron acquisition, and plant stress adaptation), Top 100 Genera with Oxygen Metabolism, pathogen screen (including but not limited to bacterial and fungal pathogens and viruses), number of microbes and viruses, relative abundance of genera, pathogen number and percentage, virus percentage, bacteria to fungal ratio anaerobic fungal number and percentage, aerobic fungal number and percentage, anaerobic bacteria number and percentage, aerobic bacteria number and percentage, ectomycorrhizal to arbuscular ratio, facultative organisms, obligate anaerobic organisms, microaerophilic organisms, and any combination thereof.

In some embodiments, a report may be generated summarizing results of analyses described herein. The report may contain results of analysis acquired through shotgun metagenomics. Reads acquired through shotgun metagenomics may be normalized for read depth and counted. Community members, soil function ratings, and soil risk ratings may be calculated by comparing the relative abundance of species or genes in the sample to a large data set of other agricultural soils to generate a percentile, which represents the value in a normal distribution that has a specific percentage of observations below it. Results may be binned into quintiles to generate ratings (i.e., 0-20% rated as very low, 20-40% rated as low, 40-60% rated as medium, 60-80% rated as high, and 80-100% rated as very high). Species contributing to the reads obtained through shotgun sequencing may be identified. The species may make up a soil microbiome. Soil microbiome richness and biodiversity supports multiple soil functions such as nutrient cycling and pathogen resistance. Observed species score correlates with soil microbiome richness and diversity. Community evenness score correlates with total number of species in a community and measures distribution of species in a community. An increase in nutrient cycling score, community structure score, and/or total score may indicate success or benefit derived from the implementation of a beneficial plant management technique or the use of a biological input. Roots may associate with mycorrhizal fungi. Fungi may enhance nutrient access, tolerance to stress, and promote plant growth. Stress may comprise soil-borne pathogens, drought, and salinity. Cover cropping and no-till practices may increase diversity and mycorrhizal associations with plants. Fungal to bacterial ratio (F/B ratio) is a measurement of soil disturbance intensity and frequency. This result may be represented in a report as a ratio of reads classified as fungal versus bacterial. Rare biosphere measurement represents number of rare and unclassified microbes in a soil sample.

In some embodiments, nitrogen cycling score may be calculated. The measurement of functional genes involved in nitrogen (N) transformation in soil has been used to predict N cycling process rates and provides insight into N-cycling microbial population response to N fertilization. This soil analysis measures the relative abundances of genes involved in three important N-cycling processes: The Nitrification rating measures the genetic potential for soil microbes to transform ammonium (NH4+) into nitrate (NO3−). Nitrate is the form of nitrogen that is most susceptible to nitrogen leaching and gaseous nitrous oxide production. Decreasing nitrification through management is desirable to decrease nitrogen loss and increase fertilizer efficiency. Strategies to control nitrification include timing of fertilization to coincide with rapid plant uptake, use of slow-release fertilizers, and biological nitrification inhibitors. The Denitrification rating measures the genetic potential for soil microbes to transform nitrate (NO3−) to nitrogenous gas (N2/N2O) and results in nitrogen loss. This process occurs during reduced oxygen availability caused by wet or poorly drained soil, compaction, high temperatures and excessive decomposable organic matter. Denitrification can be controlled through improved soil drainage, use of cover crops and residues, and controlling irrigation to provide less and more frequent applications of water. The use of nitrification inhibitors has also been shown to lower denitrification. The N-fixation rating measures the genetic potential for soil microbes to transform atmospheric nitrogen (N2) into plant available ammonia (NH4+). Nitrogen fixation is one of the major sources of nitrogen for plants. The use of cover crops and biological fertilizers containing N-fixing microbes are strategies to improve N-fixation.

In some embodiments, phosphorous cycling score may be calculated. Soil microbes are effective at releasing plant available phosphorous (P) from the soil through solubilization of insoluble inorganic P and mineralization of insoluble organic P. Solubilization occurs through the microbial release of organic acids which are generated from central metabolism while P mineralization occurs via the activity of various microbial enzymes. This soil analysis measures the relative abundance of microbial genes involved with P mineralization. Crop rotation and application of lime and compost may improve P cycling.

In some embodiments, polymer degradation score may be calculated. Polymer degradation by soil microbes plays an important role in nutrient cycling by ensuring complex compounds in the soil are readily degraded into plant available nutrients. The relative abundance of various catalytic enzymes that degrade complex carbohydrates can indicate the complexity of macromolecular material available to be digested to sustain microbial and plant growth.

In some embodiments, drought resistance score may be calculated. The relative abundance of trehalose synthesizing genes in soil may increase the drought tolerance of both bacteria and plants inoculated with trehalose synthesizing bacteria. In some embodiments, synthesis of trehalose by soil microbes corresponds with the capacity to withstand osmotic stress during droughts.

In some embodiments, soil disturbance score may be calculated. Soil disturbances such as tilling, fertilization and drought may cause significant shifts in microbial community structure that correlate with biological soil health. Heavily disturbed soils may correlate with lower health scores and contain microbes with larger genome sizes while less disturbed soils correlate with higher health scores and contain microbes with smaller genomes. To determine the disturbance rating, community weighted genome size may be calculated and rated against a reference population.

In some embodiments, community composition score may be calculated. The phylum represents a broad group of organisms with a certain degree of similarity and is a taxonomic classification that is placed below “Kingdom” and above “Class”. The top bacterial and fungal phyla in this soil sample may be listed and ranked according to percent abundance. In addition, the top 20 bacterial/archaea and fungi species may be listed and ranked according to percent abundance.

In some embodiments, threat overview or threat overview score may be assessed. Common soilborne pathogens may include Fusarium, Pythium, Rhizoctonia, Phytophthora, Rhizopus, and Verticillium. A soil sample may be analyzed via shallow shotgun DNA sequencing and screened for 170 known bacterial and fungal soilborne pathogens. Crop rotation may reduce pathogen burden while elevated soil carbon, particularly from manure application, may increase pathogen burden.

In some embodiments, the methods disclosed herein may further comprise generating a score based on analysis of a sample. Said score may take into account one or more factors selected from observed genera, community evenness, mycorrhizae, fungal/bacteria ratio (F/B ratio), rare biosphere, nitrification, denitrification, nitrogen fixation, phosphorous cycling, polymer degradation, drought resistance, disturbance, total score, and any combination thereof. In some embodiments, analysis of genes disclosed in Tables 1-32 may be used to generate said score.

In some embodiments, nutrients may comprise but are not limited to one or more of nitrogen, oxygen, phosphorous, calcium, iron, sodium, potassium, sulfur, magnesium, chlorine, hydrogen, iron, boron, manganese, zinc, copper, nickel, and molybdenum. Cycling of nutrients may comprise but is not limited to one or more of the following processes: sorption, desorption, precipitation, dissolution, plant uptake, tile flow, sedimentation, resuspension, erosion, runoff, leaching, mineralization, immobilization, fixation, assimilation, evaporation, respiration, recycling, lysis, transpiration, combustion, and deposition.

In some embodiments, pathogen species can include but are not limited to Pythium spp., Verticillium dahliae, Rhizoctonia solani, Phytophthora spp., Sclerotinia sclerotiorum, Macrophomina phaseolina, Rhizoctonia spp., Botrytis cinerea, Armillaria spp., Agrobacterium tumefaciens, Pythium aphanidermatum, Fusarium spp., Alternaria alternata, Alternaria spp., Fusarium graminearum, Meloidogyne incognita, Colletotrichum spp., Colletotrichum graminicola, Pseudomonas syringae, Fusarium solani, Pythium irregulare, Meloidogyne arenaria, Fusarium avenaceum, Meloidogyne javanica, Meloidogyne hapla, Pseudomonas syringae pv. syringae, Rhizopus spp., Colletotrichum gloeosporioides, Fusarium oxysporum, Phytophthora cactorum, Rhizoctonia solani AG-3 Rhs1AP, Phytophthora parasitica, Sclerotinia spp., Cercospora spp., Plasmodiophora brassicae, Monosporascus cannonballus, Fusarium culmorum, Verticillium spp., Thielaviopsis paradoxa, Phytophthora capsici, Ralstonia solanacearum, Alternaria brassicicola, Fusarium phaseoli, Xanthomonas campestris pv. campestris, Bipolaris sorokiniana, Phoma spp., Aspergillus spp., Fusarium verticillioides, Pseudomonas syringae pv. apii, Sclerotium cepivorum, Pythium arrhenomanes, Taphrina deformans, Bipolaris spp., Clavibacter michiganensis, Fusarium oxysporun f. sp. cepae, Phytophthora fragariae, Botrytis spp., Gaeumannomyces tritici, Fusarium sambucinum, Aspergillus flavus, Thielaviopsis basicola, Phytophthora cinnamomi, Phytophthora cryptogea, Pectobacterium carotovorum subsp. carotovorum, Pseudomonas syringae pv. atrofaciens, Thielaviopsis spp., Fusarium oxysporun f. sp. fragariae, Fusarium oxysporum f. sp. vasinfectum, Pseudomonas amygdali pv. mori, Cladosporium cladosporioides, Pseudomonas syringae pv. tagetis, Fusarium proliferatum, Fusarium oxysporum f. sp. melonis, Ceratocystis fimbriata, Ustilago hordei, Ascochyta rabiei, Pseudomonas coronafaciens pv. oryzae, Ascochyta spp., Pseudomonas syringae pv. pisi, Fusarium tricinctum, Fusarium oxysporun f. sp. conglutinans, Fusarium tricinctum species complex, Sarocladium oryzae, Albugo candida, Streptomyces scabiei, Fusarium virguliforme, Trametes versicolor, Dactylonectria spp., Burkholderia cepacia, Ganoderma spp., Pseudomonas cannabina, Helminthosporium spp., Binucleate Rhizoctonia, Heterodera glycines, Pseudomonas syringae pv. coryli, Ilyonectria spp., Pseudomonas syringae pv. spinaceae, Laetiporus sulphureus, Colletotrichum coccodes, Leptosphaeria maculans ‘brassicae’ group, Ramularia spp., Macrophomina phaseoli, Fusarium oxysporum f. sp. lactucae, Diaporthe, Fusarium oxysporum f. sp. lycopersici, Magnaporthe oryzae, Spongospora subterranea, Aspergillus niger, Fusarium oxysporun f. sp. pisi, Meloidogyne floridensis, Trametes hirsuta, Diaporthe spp., Trichoderma koningii, Diplodia spp., Verticillium albo-atrum, Agrobacterium rhizogenes, Plasmopara halstedii, Meloidogyne spp., Pseudomonas avellanae, Enterobacter cloacae, Pseudomonas cichorii, Mucor spp., Pseudomonas savastanoi pv. phaseolicola, Neofusicoccum spp., Colletotrichum acutatum, Pantoea ananatis, Pseudomonas syringae pv. coriandricola, Pantoea stewartii, Pseudomonas syringae pv. helianthi, Pectobacterium carotovorum, Pseudomonas syringae pv. rhaphiolepidis, Verticillium longisporum, Fusarium oxysporum f. sp. apii, Enterobacter cloacae complex, Bipolaris oryzae, Epicoccum nigrum, Fusarium oxysporum f. sp. ciceris, Xanthomonas translucens pv. translucens, Fusarium oxysporun f. sp. cucumerinum, Xylaria spp., Botryosphaeria spp., Phaeoacremonium spp., Agrobacterium rubi, Phaeomoniella spp., Rhizopus stolonifer, Phellinus spp., Schizophyllum commune, Phoma herbarum, Fusarium oxysporum f. sp. medicaginis, Eremothecium coryli, scn:scn, Erwinia amylovora, Streptomyces ipomoeae, Phytophthora cambivora, Fusarium oxysporun f. sp. niveum, Eutypa lata, Fusarium oxysporum f. sp. radicis-cucumerinum, Eutypa spp., Tilletia spp., Fomitiporia spp., Trametes spp., Aphanomyces spp., Trichoderma harzianum, Phytophthora nicotianae, Uromyces spp., Claviceps purpurea, Ustilago maydis, Phytophthora sojae, Verticillium alfalfae, Athelia rolfsii, Fusarium oxysporum f. sp. radicis-lycopersici, Alternaria, Xanthomonas albilineans, Penicillium chrysogenum, Xanthomonas campestris pv. vesicatoria, Penicillium oxalicum, Xanthomonas vasicola pv. vasculorum, Penicillium spp., Ceratocystis adiposa, and Peronospora spp.

In some embodiments, microbial diversity can be assess by approaches analyzing the intergenic region between 16S ribosomal RNA and 23S ribosomal RNA. In some embodiments, microbial diversity can be further described by approaches analyzing the 16S ribosomal RNA.

In some embodiments, microbial diversity can be further described by approaches analyzing the 23S ribosomal RNA. In some embodiments, primers can be designed to specifically amplify any identified variable regions in a microbe or similar distinguishing genetic element. In some embodiments, microbial diversity can be assessed by approaches using shotgun metagenomic sequencing at 3 million, 10 million, or 25 million or more read depths.

In some embodiments, a library can be prepared from the genetic material. In some embodiments, the library can be prepared by use of amplification, shotgun, whole molecule techniques among others. In some embodiments, amplification to add adapters for sequencing, and/or barcoding for sequences can be performed. In some embodiments, shotgun by sonication and/or enzymatic cleavage can be performed. In some embodiments, whole molecules can be used to sequence all DNA in a sample.

In some embodiments, sequencing can be performed. In some embodiments, the sequencing is performed with a high-throughput system, such as for example 454, Illumina, PacBio, or IonTorrent, or Nanopore. In some embodiments, sequencing is performed via shotgun metagenomic sequencing.

In some embodiments, sequence analysis is prepared. In some embodiments, this analysis can be performed using tools such as QIIME Analysis Pipeline, Machine learning, and UniFrac. In some embodiments, this analysis can be performed using Kraken, Kraken1, Kraken2, Diamond or SourMash. In some embodiments, a sequence from the sample is tagged for example via a barcode, for among other things quality control of sequence data.

In some embodiments, the processing and analysis can further involve matching the sequences to the samples. In some embodiments, the method can further comprise aligning the sequences to each other, and/or using the aligned sequences to build a phylogenetic tree. In some embodiments, the method can further comprise distilling the data to form an n-dimensional plot and/or a two or three dimensional plot or other graphical displays, including displays of the results of machine learning and multivariate statistical routines. In some embodiments, the method can further comprise using the two or three-dimensional plot or other graphical displays to visualize patterns of the microbial communities in a particular sample over time and geographic space.

In some embodiments, sequencing method disclosed herein can comprise: (a) sequencing the target nucleic acid to produce a primary target nucleic acid sequence that comprises one or more sequences of interest; (b) synthesizing a plurality of target-specific oligonucleotides, wherein each of said plurality of target-specific oligonucleotides corresponds to at least one of the sequences of interest; (c) providing a library of fragments of the target nucleic acid (or constructs that comprise such fragments and that can further comprise, for example, adaptors and other sequences as described below) that hybridize to the plurality of target-specific oligonucleotides; and (d) sequencing the library of fragments (or constructs that comprise such fragments) to produce a secondary target nucleic acid sequence. The target nucleic acid can be any nucleic acid, including but not limited to genomic DNA from any organism, such as, for example, genomes of organisms such as bacteria, fungi (e.g., yeast) or mammals.

In saying that the plurality of target-specific oligonucleotides corresponds to at least one of the sequences of interest, it is meant that such target-specific oligonucleotides are designed to hybridize to the target nucleic acid in proximity to, including but not limited to, adjacent to, the sequence of interest such that there is a high likelihood that a fragment of the target nucleic acid that hybridizes to such an oligonucleotides will include the sequence of interest. In some embodiments, such target-specific oligonucleotides are therefore useful for hybrid capture methods to produce a library of fragments enriched for such sequences of interest, as sequencing primers for sequencing the sequence of interest, as amplification primers for amplifying the sequence of interest, or for other purposes.

In some embodiments, the method of the present disclosure comprises receiving metadata describing a soil or environment sample, where the metadata indicates one or more types of crops/plants grown in a geographical location having the soil or environment sample. In some embodiments, the method can further include determining nucleic acid sequence reads of the soil or environment sample. In some embodiments, the method can further include determining, for each nucleic acid sequence read of at least a subset of the nucleic acid sequence reads, functional descriptors of the genes represented in nucleic acid sequence reads. In some embodiments, the method can further include determining reference metrics of soil or environment samples from geographical locations in which the one or more types of crops/plants were grown. In some embodiments, the method can further include determining a metric of the soil or environment sample using functional descriptors and reference metrics. In some embodiments, the method can further include transmitting the metric to a client device for display on a user interface.

In some embodiments, determining the metric of the soil or environment sample comprises determining a value of a soil or environment health indicator of the soil or environment sample using the functional descriptors. In some embodiments, the method can further include determining a distribution of values of the soil or environment health indicator for the soil or environment samples using the reference metrics. In some embodiments, the method can further include determining a percentile of the value with respect to the distribution of values.

In some embodiments, determining the functional descriptors comprises determining a plurality of microbial functional genes (also referred to herein as functional genes or microbial genes) in the soil or environment sample. In some embodiments, the method can further include determining, for each of the plurality of genes, a count of the genes in the soil or environment sample. In some embodiments, the method can further include normalizing these counts using a total count of genes in the soil or environment sample or by other methods such as single copy marker genes, sequence read depth, etc.

In some embodiments, determining the nucleic acid sequence reads of the soil or environment sample can comprise extracting microbial material from the soil or environment sample. In some embodiments, the method further includes generating nucleic acid sequence reads of the microbial material. In some embodiments, the method further includes filtering the nucleic acid sequence reads by read quality scores.

In come embodiments, a method disclosed herein can include obtaining a soil or environment sample from a geographical location. The method can further include receiving metadata indicating the geographical location. In some embodiments, the method can further include determining a plurality of functional genes in the soil or environment sample. In some embodiments, the method can further include determining, for each of the plurality of functional genes, a measure of the functional gene in the soil or environment sample. In some embodiments, the method can further include determining functional descriptors of genes represented in the soil or environment sample using the measures of the functional genes. In some embodiments, the method can further include determining reference metrics of soil or environment samples from geographical locations within a threshold distance of the geographical location. In some embodiments, the method can further include determining a metric of the soil or environment sample using the functional descriptors and the reference metrics. In some embodiments, the method can further include transmitting the metric to a client device for display on a user interface.

### Devices

Disclosed herein are devices for use in the systems and methods of the present disclosure. In some embodiments, a device is used to collect a sample for analysis of the rhizobiome. In some embodiments, the sample for analysis of the rhizobiome is collected without a device. In some embodiments, the device comprises an input. In some embodiments, the input comprises an attractant. In some embodiments, the input comprises a plant seed. In some embodiments, the input comprises a plant. In some embodiments, the input comprises a plant root. In some embodiments, the input comprises a fertilizer. In some embodiments, the input comprises a biofertilizer. In some embodiments, the input comprises a biostimulant. In some embodiments, the input comprises a nutrient composition. In some embodiments, the input comprises plant food. In some embodiments, the device comprises a sensor. In some embodiments, the sensor detects humidity. In some embodiments, the sensor detects moisture. In some embodiments, the sensor detects pH.

An aspect of the present disclosure is the collection of a sample of a rhizosphere. In some embodiments, a device disclosed herein can be used in some embodiments to collect the sample. In some embodiments, the sample can comprise one or more of the following components: soil, liquid, paper, seed, plant, or microorganisms. In some embodiments, the components of the sample can be analyzed to extract their genetic material. In some embodiments, the genetic material can be DNA and/or RNA. In some embodiments, the genetic material can be sequenced to determine one or more of gene expression, categorization or identification of microorganisms, presence of pests or disease, plant variety, plant health, soil health, or a plant attribute disclosed herein.

In some embodiments, a device disclosed herein can be placed in soil or an environment to capture early forming rhizosphere during seed germination and early growth. After germination and/or early growth period elapses, the device can be removed from the environment of a seed or plant for the collection of genetic material contained within said device. In some embodiments, sequencing can be performed on the genetic material obtained from said device.

In some embodiments, germination can occur within or close to the device. In some embodiments, germination can cause release of exudates from a seed. In some embodiments, exudates can recruit one or more microorganisms to form a rhizosphere. In some embodiments, conditions for germination can comprise modulation of one or more of a greenhouse, a laboratory, a controlled setting, a field, soil composition, water availability, oxygen concentration, percent humidity, light, air composition, or temperature.

In some embodiments, the device can be used to collect a sample from within 100 millimeters of the roots of a plant. In some embodiments, the sample can be collected from within less than about 5, 10, 15, 20, 25, 30, 40, 50, 60, 70, 80, 90, or 100 millimeters of said root of said plant. In some embodiments, the sample can be collected from less than about 0.1, 0.2, 0.3, 0.4, 0.5, 0.6, 0.7, 0.8, 0.9, 1.0, 1.5, 2.0, 2.5, or 3.0 millimeters of said root of said plant. In some embodiments, obtaining a sample can be performed one or more times. In some embodiments, obtaining a sample can be carried out over multiple days. In some embodiments, obtaining a sample can be performed before or after germination.

In some embodiments, the device can be a small permeable tube. In some embodiments, the rhizosphere can form inside of the device. In some embodiments, the device can be used to capture the root-soil interface. In some embodiments, the device is reusable. In some embodiments, the device is compostable. In some embodiments, the device is disposable. In some embodiments, the device can comprise plastic, paper, metal, glass, or any combination thereof. In some embodiments, the device is cylindrical. In some embodiments, the device has a removable cap or lid. In some embodiments, the device can be filled with soil. In some embodiments, the device can contain one or more seeds or attractant disclosed herein. In some embodiment, the device can be planted within soil. In some embodiments, the device can be submerged beneath the surface of a volume of soil. In some embodiments, the device can be submerged at a depth of at least one inch, at least 5 inches, at least 10 inches, at least 15 inches, at least 20 inches, at least 25 inches, at least 30 inches, at least 35 inches, at least 40 inches, at least 45 inches, at least 50 inches, at least 55 inches, or at least 60 inches. In some embodiments, the device can contain a seed, plant or attractant for at least 1 one day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18, at least 19 days, at least 20 days, at least 21 days, at least 30 days, at least 2 months, at least 3 months, at least 4 months, at least 5 months, at least 6 months, at least 7 months, at least 8 months, at least 9 months, at least 10 months, at least 11 months, or at least 12 months.

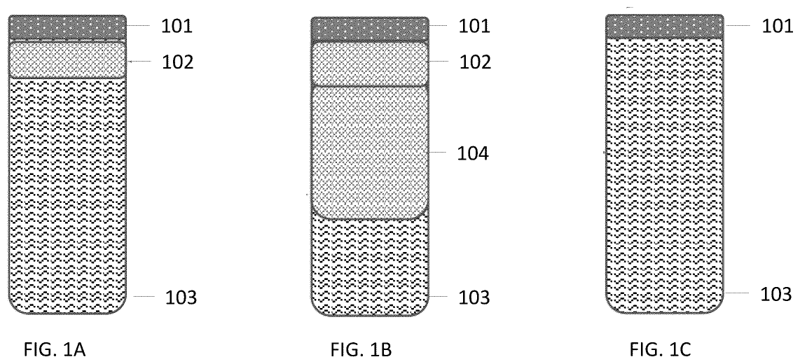

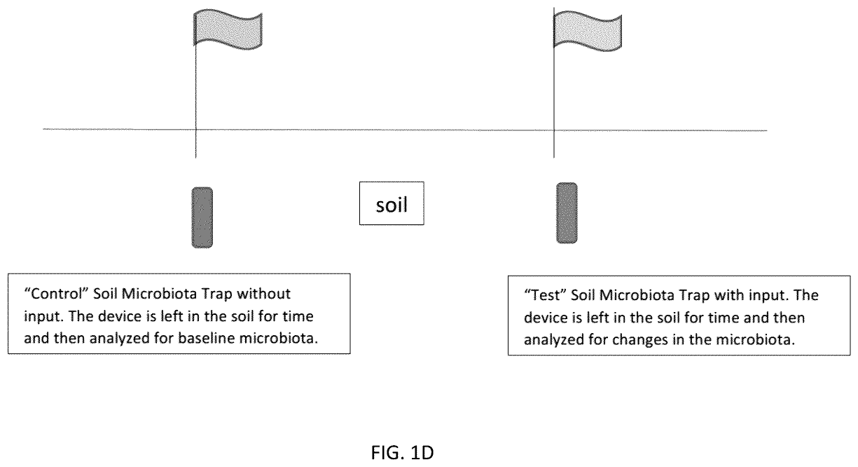

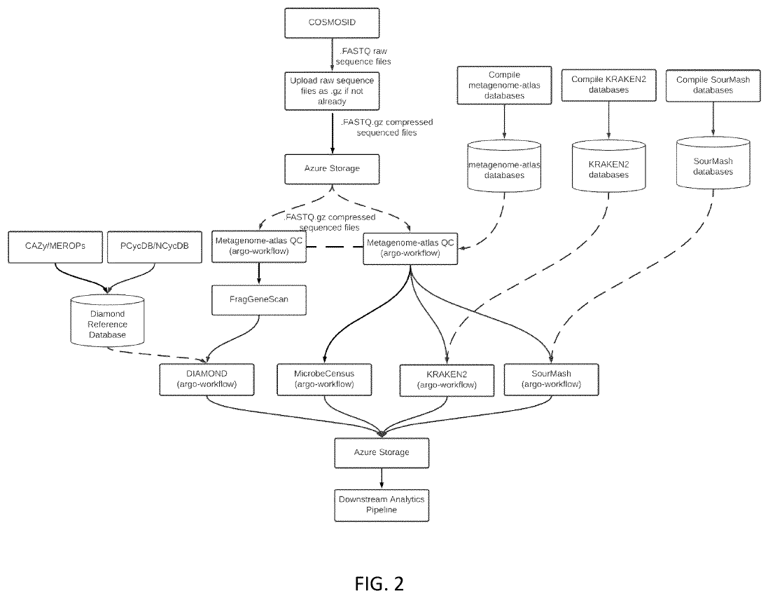

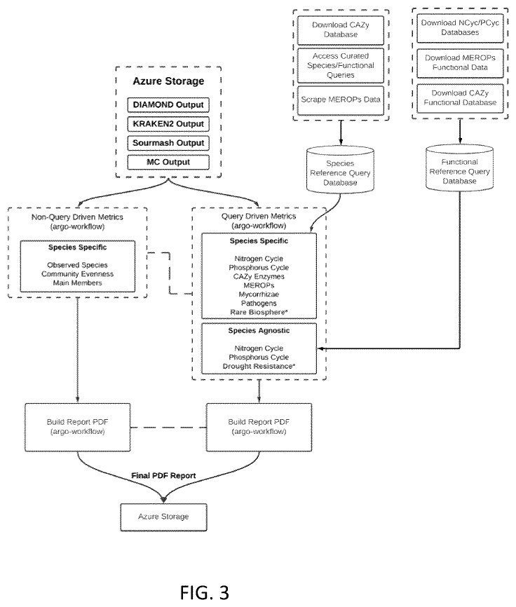

In some embodiments, the device comprises a lid or a top as shown in FIGS. 1A, FIG. 1B and FIG. 1C at 101. In some embodiments, the lid or top 101 is attached to the body of the device. In some embodiments, the lid or top 101 snaps on. In some embodiments, the lid or top 101 is a screw top. In some embodiments, the top or lid 101 is perforated. In some embodiments, the bottom of the device is perforated as shown in FIG. 1A, FIG. 1B and FIG. 1C at 103. In some embodiments, the device comprises an input container as shown in FIG. 1A and FIG. 1B at 102. In some embodiments, the input container 102 has solid sides. In some embodiments, the input container 102 has a perforated bottom. In some embodiments, the device comprises perforated sides after the input container 102 as depicted in FIG. 1A. In some embodiments, the device comprises perforated sides as depicted in FIG. 1C. In some embodiments, the device comprises as additional solid section to the sides following the input container 102 as depicted in FIG. 1B. In some embodiments, a control and a test device can be used as shown in FIG. 1D. In this depicted embodiment, the control device can be without inputs and the test device can have inputs and are placed in the soil. In this depicted embodiment, the devices are marked. In this depicted embodiment, the control device has a different marking than the test device. In some embodiments, the device comprising the input, when the soil is analyze comprises more microorganisms as compared to the control device.

In some embodiments, analysis of the sample may include any number of analytical techniques including metagenomics, metabolomics, meta transcriptomics, immunoassays, chemical analysis. In some embodiments, following analysis, appropriate data analytics can be applied to evaluate changes in the molecular, chemical, taxonomic, etc. composition of the test device(s) versus the control device. In some embodiments, certain analytical techniques, such as metagenomics, can be used to evaluate changes in the soil microbiota to make predictions on input performance. In some embodiments, functional attributes that can be evaluated can include changes in biodiversity, community evenness, changes in mycorrhizae composition, changes in composition of legume-nodulating bacteria, changes in function gene composition (i.e., nitrogen cycling, calcium transport, phosphate cycling, polymer degradation, etc.), changes in pathogen levels, etc.

In some embodiments, analysis of a sample disclosed herein can identify a biomarker of plant health, soil health, nitrogen mineralization potential, carbon mineralization potential, phosphorus mineralization potential or a plant attribute disclosed herein. Biomarkers may be identified in bulk soil that may correlate with biomarkers identified in the early forming rhizosphere. Biomarkers may predict crop performance. Biomarkers may predict effects of one or more biological inputs on crop performance. Biomarkers may be combined with other metadata including but not limited to weather, soil texture, fertility data, crop type, satellite imagery, and any combination thereof in order to predict crop yield or the effect of one or more biological inputs on crop yield. Biological inputs may include but are not limited to biological fertilizers, probiotics, biological stimulants, prebiotics, biological control agents, and any combination thereof. Biological inputs may be applied to seeds directly. Biological inputs may be applied to soil. Biological inputs may be applied before or after germination. Biomarkers may be used to inform seed selection.

Analysis and methods described herein may be applied to the rhizosphere, endosphere, stems, and/or leaves of a plant.

### Systems

An aspect of the present disclosure is a system used to carry out the method of the present disclosure. In some embodiments, one or more processors can execute instructions stored by a non-transitory computer-readable storage medium to control a computer system to perform steps of any of the above methods. In some embodiments, a system can compromise a sampling tube for obtaining a soil or environment sample from a geographical location. In some embodiments, the system can further include one or more processors and a memory, the memory storing computer program instructions that when executed by the one or more processors cause the one or more processors to perform steps of any of the above methods.

The system for performing the method can comprise a controller, at least one processor, and an internet connection. In some embodiments, the system can further comprise a sequencer.

In some embodiments, the files generated by the sequencing machine are used as input for a pipeline (FIG. 2). In some embodiments, the sequencing file are received by a computing system. In some embodiments, the sequencing file are run through a quality control program. In some embodiments, the quality control program removes PCR duplicates form the reads of the sequencing file. In some embodiments, the quality control program removes or trims low quality reads from the sequencing file. In some embodiments, the quality control program removes reads that are from the host. In some embodiments, the host is a plant. In some embodiments, the quality control program removes common contaminates in the reads of the sequencing file.

In some embodiments, the quality-controlled sequencing file is run through a program to identify genes. In some embodiments, the quality-controlled sequencing file is run through a program to identify coding regions. In some embodiments, these genes are short, as in they do not have a lot of nucleic acids. In some embodiments, these genes are error-prone. In some embodiments, the quality-controlled sequencing file is run through a program to extract genes. In some embodiments, the quality-controlled sequencing file is run through a program to extract coding regions.

In some embodiments, the output of the program to identify and/or extract genes and/or coding regions is used with a program that performs sequence alignment. In some embodiments, the output of the program to identify and/or extract genes and/or coding regions is used with a program that performs protein sequence alignment. In some embodiments, the sequence alignment is performed with a reference database. In some embodiments, the sequence alignment is performed to quantify a functional profile. In some embodiments, the protein sequence alignment is performed with a reference database. In some embodiments, the protein sequence alignment is performed to quantify a functional profile.

In some embodiments, the sequencing files are run through a program to apply taxonomic labels. In some embodiments, the taxonomic labels can identify the microorganisms present in a sample. In some embodiments, the taxonomic labels are applied to metagenomic DNA sequences. In some embodiments, the taxonomic labels are applied through a comparison to reference libraries. In some embodiments, the reference library is Silva. In some embodiments, the reference library is GreenGenes.

In some embodiments, the sequencing files are run through a program to determine an approximate average genome size. In some embodiments, the average genome size is approximated as a mean of quantifying disturbance rating. In some embodiments, pathogens are detected in the sequencing file by running them through a program that compares the sequencing file to pathogenic reference libraries.

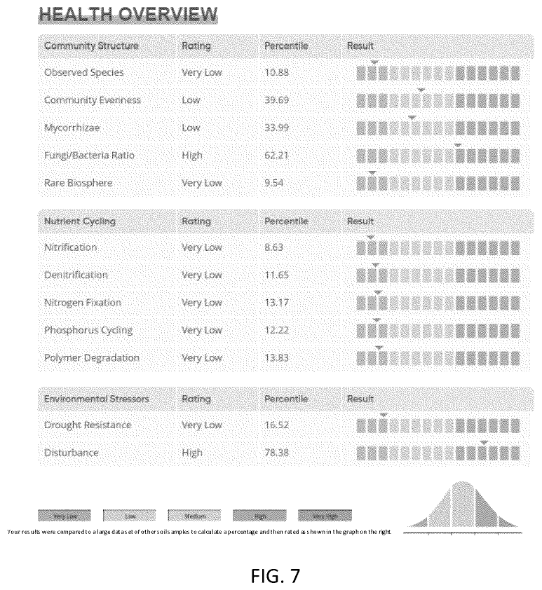

In some embodiments, the results and/or output of one or more of the above referenced programs are used to generate a report (FIG. 7). In some embodiments, the report is a PDF file. In some embodiments, the report can show one or more results for community structure. In some embodiments, the report can show one or more results for nutrient cycling, environmental stressors, soil attribute, plant attribute, environment attribute, or treatment recommendation disclosed herein. In some embodiments, the report can show one or more plant management plan and/or one or more implementation plan. Nutrients may comprise but are not limited to one or more of nitrogen, oxygen, phosphorous, calcium, iron, sodium, potassium, sulfur, magnesium, chlorine, hydrogen, iron, boron, manganese, zinc, copper, nickel, and molybdenum. Cycling of nutrients may comprise but is not limited to one or more of the following processes: sorption, desorption, precipitation, dissolution, plant uptake, tile flow, sedimentation, resuspension, erosion, runoff, leaching, mineralization, immobilization, fixation, assimilation, evaporation, respiration, recycling, lysis, transpiration, combustion, and deposition.

Although the input data, data processing, and output data are described herein in a pipeline, the data can be processed in a variety of ways. In some embodiments, the data is processed manually. In some embodiments, the data has undergone preprocessing before being used in the systems disclosed herein. In some embodiments, only part of the data processing described herein is performed.

The above-described embodiments can be implemented in any of numerous ways. For example, embodiments can be implemented using hardware, software or a combination thereof. In some embodiments, when implemented in software, the software code can be executed on any suitable processor or collection of processors, whether provided in a single computer or distributed among multiple computers.

Further, it should be appreciated that a computer can be embodied in any of a number of forms, such as a rack-mounted computer, a desktop computer, a laptop computer, or a tablet computer. Additionally, a computer can be embedded in a device not generally regarded as a computer but with suitable processing capabilities, including a Personal Digital Assistant (PDA), a smart phone or any other suitable portable or fixed electronic device.

In some embodiments, a computer can have one or more input and output devices. In some embodiments, these devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer can receive input information through speech recognition or in other audible format.

Such computers can be interconnected by one or more networks in any suitable form, including a local area network or a wide area network, such as an enterprise network, and intelligent network (IN) or the Internet. Such networks can be based on any suitable technology and may operate according to any suitable protocol and may include wireless networks, wired networks or fiber optic networks.

The various methods or processes outlined herein can be coded as software that is executable on one or more processors that employ any one of a variety of operating systems or platforms. Additionally, such software can be written using any of a number of suitable programming languages and/or programming or scripting tools, and also can be compiled as executable machine language code or intermediate code that is executed on a framework or virtual machine.

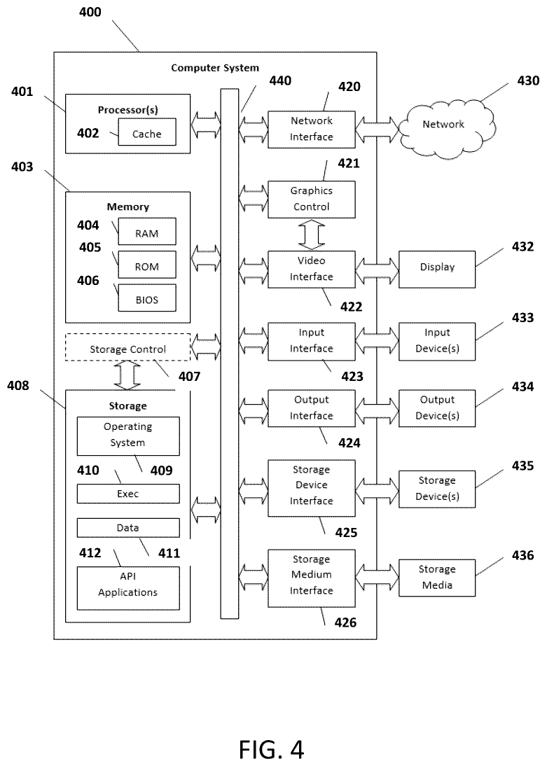

Referring to FIG. 4, a block diagram is shown depicting an exemplary machine that includes a computer system 400 (e.g., a processing or computing system) within which a set of instructions can execute for causing a device to perform or execute any one or more of the aspects and/or methodologies for static code scheduling of the present disclosure. The components in FIG. 4 are examples only and do not limit the scope of use or functionality of any hardware, software, embedded logic component, or a combination of two or more such components implementing particular embodiments.

In some embodiments, computer system 400 may include one or more processors 401, a memory 403, and a storage 408 that communicate with each other, and with other components, via a bus 440. The bus 440 may also link a display 432, one or more input devices 433 (which may, for example, include a keypad, a keyboard, a mouse, a stylus, etc.), one or more output devices 434, one or more storage devices 435, and various tangible storage media 436. All of these elements may interface directly or via one or more interfaces or adaptors to the bus 440. For instance, the various tangible storage media 436 can interface with the bus 440 via storage medium interface 426. Computer system 400 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

In some embodiments, computer system 400 includes one or more processor(s) 401 (e.g., central processing units (CPUs), general purpose graphics processing units (GPGPUs), or quantum processing units (QPUs)) that carry out functions. Processor(s) 401 optionally contains a cache memory unit 402 for temporary local storage of instructions, data, or computer addresses. Processor(s) 401 are configured to assist in execution of computer readable instructions. Computer system 400 may provide functionality for the components depicted in FIG. 4 as a result of the processor(s) 401 executing non-transitory, processor-executable instructions embodied in one or more tangible computer-readable storage media, such as memory 403, storage 408, storage devices 435, and/or storage medium 436. The computer-readable media may store software that implements particular embodiments, and processor(s) 401 may execute the software. Memory 403 may read the software from one or more other computer-readable media (such as mass storage device(s) 435, 436) or from one or more other sources through a suitable interface, such as network interface 420. The software may cause processor(s) 401 to carry out one or more processes or one or more steps of one or more processes described or illustrated herein. Carrying out such processes or steps may include defining data structures stored in memory 403 and modifying the data structures as directed by the software.

In some embodiments the memory 403 may include various components (e.g., machine readable media) including, but not limited to, a random access memory component (e.g., RAM 404) (e.g., static RAM (SRAM), dynamic RAM (DRAM), ferroelectric random access memory (FRAM), phase-change random access memory (PRAM), etc.), a read-only memory component (e.g., ROM 405), and any combinations thereof. ROM 405 may act to communicate data and instructions unidirectionally to processor(s) 401, and RAM 404 may act to communicate data and instructions bidirectionally with processor(s) 401. ROM 405 and RAM 404 may include any suitable tangible computer-readable media described below. In one example, a basic input/output system 406 (BIOS), including basic routines that help to transfer information between elements within computer system 400, such as during start-up, may be stored in the memory 403.

In some embodiments fixed storage 408 is connected bidirectionally to processor(s) 401, optionally through storage control unit 407. Fixed storage 408 provides additional data storage capacity and may also include any suitable tangible computer-readable media described herein. Storage 408 may be used to store operating system 409, executable(s) 410, data 411, applications 412 (application programs), and the like. Storage 408 can also include an optical disk drive, a solid-state memory device (e.g., flash-based systems), or a combination of any of the above. Information in storage 408 may, in appropriate cases, be incorporated as virtual memory in memory 403.

In some embodiments, storage device(s) 435 may be removably interfaced with computer system 400 (e.g., via an external port connector (not shown)) via a storage device interface 425. Particularly, storage device(s) 435 and an associated machine-readable medium may provide non-volatile and/or volatile storage of machine-readable instructions, data structures, program modules, and/or other data for the computer system 400. In one example, software may reside, completely or partially, within a machine-readable medium on storage device(s) 435. In another example, software may reside, completely or partially, within processor(s) 401.

In some embodiments, bus 440 connects a wide variety of subsystems. Herein, reference to a bus may encompass one or more digital signal lines serving a common function, where appropriate. Bus 440 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

In some embodiments, computer system 400 may also include an input device 433. In one example, a user of computer system 400 may enter commands and/or other information into computer system 400 via input device(s) 433. Examples of an input device(s) 433 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 433 may be interfaced to bus 440 via any of a variety of input interfaces 423 (e.g., input interface 423) including, but not limited to, serial, parallel, game port, USB, FIREWIRE, THUNDERBOLT, or any combination of the above.

In particular embodiments, when computer system 400 is connected to network 430, computer system 400 may communicate with other devices, specifically mobile devices and enterprise systems, distributed computing systems, cloud storage systems, cloud computing systems, and the like, connected to network 430. Communications to and from computer system 400 may be sent through network interface 420. For example, network interface 420 may receive incoming communications (such as requests or responses from other devices) in the form of one or more packets (such as Internet Protocol (IP) packets) from network 430, and computer system 400 may store the incoming communications in memory 403 for processing. Computer system 400 may similarly store outgoing communications (such as requests or responses to other devices) in the form of one or more packets in memory 403 and communicated to network 430 from network interface 420. Processor(s) 401 may access these communication packets stored in memory 403 for processing.

Examples of the network interface 420 include, but are not limited to, a network interface card, a modem, and any combination thereof. Examples of a network 430 or network segment 430 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 430, may employ a wired and/or a wireless mode of communication. In general, any network topology may be used.

In some embodiments, information and data can be displayed through a display 432. Examples of a display 432 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 432 can interface to the processor(s) 401, memory 403, and fixed storage 408, as well as other devices, such as input device(s) 433, via the bus 440. The display 432 is linked to the bus 440 via a video interface 422, and transport of data between the display 432 and the bus 440 can be controlled via the graphics control 421. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 432, computer system 400 may include one or more other peripheral output devices 434 including, but not limited to, an audio speaker, a printer, a storage device, and any combinations thereof. Such peripheral output devices may be connected to the bus 440 via an output interface 424. Examples of an output interface 424 include, but are not limited to, a serial port, a parallel connection, a USB port, a FIREWIRE port, a THUNDERBOLT port, and any combinations thereof.

In addition or as an alternative, computer system 400 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung HomeSync®. Those of skill in the art will also recognize that suitable video game console operating systems include, by way of non-limiting examples, Sony® PS3®, Sony® PS4®, Sony® PS5®, Microsoft® Xbox 360®, Microsoft® Xbox One, Microsoft® Xbox Series X, Microsoft® Xbox Series S, Nintendo® Wii®, Nintendo® Wii U®, Nintendo® Switch™, and Ouya®.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight, Java™, and Unity®.

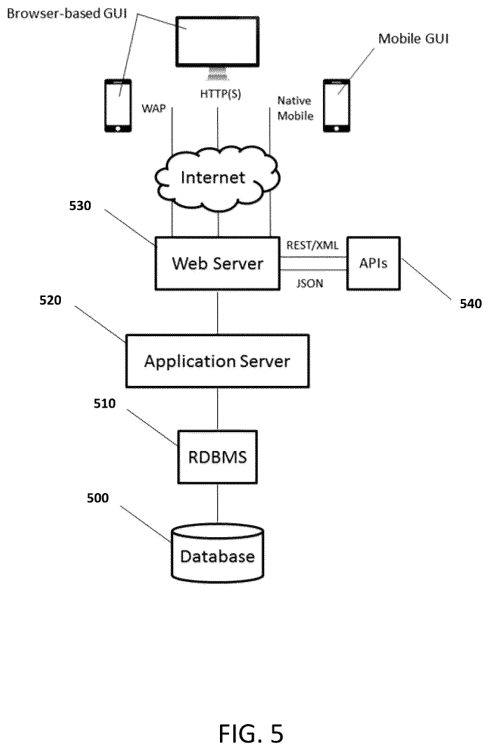

Referring to FIG. 5, in a particular embodiment, an application provision system comprises one or more databases 500 accessed by a relational database management system (RDBMS) 510. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 520 (such as Java servers, .NET servers, PHP servers, and the like) and one or more web servers 530 (such as Apache, IIS, GWS and the like). The web server(s) optionally expose one or more web services via app application programming interfaces (APIs) 540. Via a network, such as the Internet, the system provides browser-based and/or mobile native user interfaces.

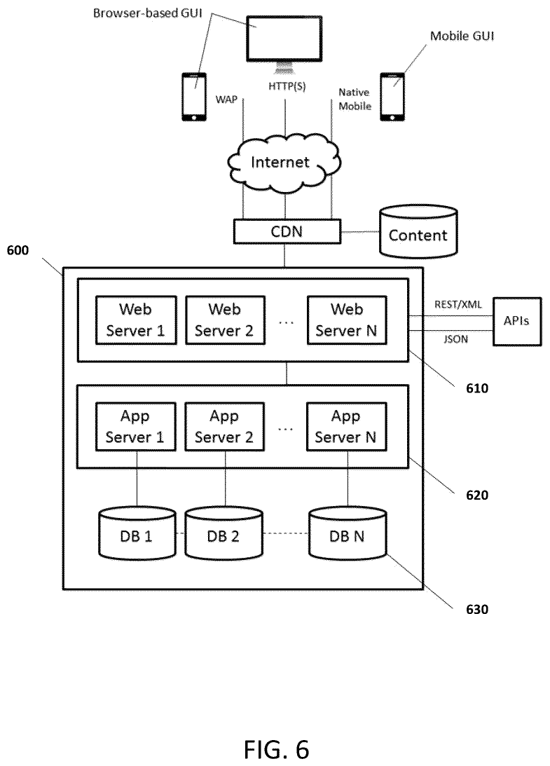

Referring to FIG. 6, in a particular embodiment, an application provision system alternatively has a distributed, cloud-based architecture 600 and comprises elastically load balanced, auto-scaling web server resources 610 and application server resources 620 as well synchronously replicated databases 630.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, Rails, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia devices, Samsung Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla Firefox® for mobile, Microsoft® Internet Explorer® Mobile, Amazon® Kindle® Basic Web, Nokia Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. In some embodiments, the software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Data Transmission

The subject matter described herein, including methods and systems for building a query, querying a network, and returning results in a table and may be configured to be performed in one or more facilities at one or more locations. Facility locations are not limited by country and include any country or territory. In some instances, one or more steps are performed in a different country than another step of the method. In some embodiments, one or more method steps involving a computer system are performed in a different country than another step of the methods provided herein. In some embodiments, data processing and storage are performed in a different country or location than one or more steps of the methods described herein. In some embodiments, one or more products or data are transferred from one or more of the facilities to one or more different facilities for analysis or further analysis. Data includes, but is not limited to, information regarding the stratification of a subject, and any data produced by the methods disclosed herein. In some embodiments of the methods and systems described herein, the subject information is compiled, and a subsequent data transmission step will transmit or store the subject information.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, performed in one facility in a particular location and corresponding data transmitted to another location.

### Business Methods Utilizing A Computer

The methods described herein can utilize one or more computers. In some embodiments, the computer may be used for managing customer and subject information such as queries, objects, properties, types, filters, tables, database management, storing data, billing, marketing, storing subject information, or a combination thereof. In some embodiments, the computer can include a monitor or other user interface for displaying data, results, billing information, marketing information (e.g. demographics), customer information, or sample information. In some embodiments, the computer may also include means for data or information input. In some embodiments, the computer may include a processing unit and fixed or removable media or a combination thereof. In some embodiments, the computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party.

The entity entering or reviewing information into a database for the purpose of one or more of the following: inventory tracking, order tracking, customer management, customer service, billing, and sales. Sample information may include, but is not limited to: customer name, unique customer identification, or any information suitable for storage in a database.

In some embodiments, the database may be accessible by a user. In some embodiments, database access may take the form of electronic communication such as a computer or telephone. The database may be accessed through an intermediary such as a customer service representative, business representative, or consultant. The availability or degree of database access may change upon payment of a fee for products and services rendered or to be rendered.

### EXAMPLES

### Example 1: Use of the Methods and Systems Disclosed Herein

A rhizosphere sample is collected from a field. The sample may comprise soil and a portion of the root of a legume plant. Genetic material is extracted from the sample. The genetic material is sequenced using next generation sequencing (NGS), specifically shotgun sequencing. The results of the shotgun sequencing reveal the absence of nitrogen fixating bacteria. A plant management technique recommends the addition of a biofertilizer comprising nitrogen fixating bacteria. The biofertilizer comprises bacteria of the genus Rhizobacter. The plant management technique is applied to the field once daily for three weeks. The implementation of the plant management technique results in increased legume crop yield as compared to a similar field that did not receive the plant management technique.

### Example 2: Collection of a Sample Using a Device

A device is used to collect a soil sample and determine a management plan. The device is semi-permeable allowing for microorganisms to move in and out. The device is buried at the base of a plant, adjacent to the roots, growing in a field. The location the device is buried at is marked to allow for easy retrieval of the device after twenty days. After twenty days have passed, the device is retrieved. Upon retrieval of the device the soil is analyzed.

Water is added to the soil to generate a water-soil slurry. DNA is isolated from the water-soil slurry. Following isolation of the DNA, the DNA is sequenced using whole genome sequencing methods on an Illumina MiSeq. The sequencing files are uploaded to a pipeline for analysis. The analysis identifies the composition of bacteria present in the soil immediately adjacent to the plant roots. Following the identification of and prevalence of the different microbes it will appear that the rhizobiome for this plant has an uneven microbial community as well as minimal amounts of phosphorus cycling bacteria.

A report is generated highlighting different aspects of the rhizobiome (FIG. 7). Additionally, a recommendation is made to apply phosphorus cycling microorganisms as well as additional microorganisms that will aid in balancing the overall microbial communities present in the rhizobiome.

### Example 3: Confirmation of Success for a Recommended Management Strategy

As described in Example 2, a farmer implements a management plan. The management plan is implemented over the entirety of a field except for a portion. Devices are buried at the base of plants adjacent to the roots at several locations in the field including the untreated section. All of the devices are marked to allow for easy retrieval after 20 days. Following the 20 days, all of the devices are retrieved and the locations of the devices are documented as they correspond to each device to aid in understanding the location of the soil that will be analyzed. The soil from the samples will be analyzed as described in Example 2.

The results for the devices buried in the treated portions of the field will show improved microbial community balance as well as a desirable level of phosphorus cycling bacteria. Furthermore, the results for the devices buried in the untreated portion of the field show the same results as the analysis in Example 2, before the implementation of the management plan.

### Example 4: Collection of a Sample without a Device

A field is assessed for overall health and a management plan is recommended. The base of the plant adjacent to roots is extracted from the soil, and the soil in contact with or in close proximity to the roots is collected. The analysis of the sample and recommendations is completed as described in Example 2.

### Example 5: Collection of a Sample in a Container

A farmer grows potted plants using a variety of soil compositions. A pot from each soil composition being used will be taken for analysis of the rhizobiome. To acquire a soil sample comprising the rhizobiome, the plant is removed from the pot and excess soil is removed to reveal the roots of the plant. The soil from around the roots is collected and the sample is processed as described in Example 2.

### Example 6: Collection of a Sample from Germination Paper

A plant is grown on germination paper with added soil microbial community extracts. After 10 days, the roots and germination paper are rinsed with water and the water is collected. DNA is isolated from the water used to rinse the roots and germination paper. The DNA is processed as described in Example 2.

### Example 7: Collection of a Rhizosphere Sample Using a Device with a Seed

A farmer plants soybean seeds in afield. A device with a seed is buried in the field. Additionally, another device without a seed is also buried in the field. Both devices are left for 20 days. After 20 days the devices are retrieved, and the details are recorded surrounding where they were located in the field, and which contained the seed. The device with the seed has soil removed from the container and processed as described in Example 2.

### Example 8: Sequence Analysis of a Rhizosphere Sample

RNA extracted from a rhizosphere sample is processed through a sequencer. The sequencing reads are analyzed by identifying genes and their relative abundance through transcriptomic analysis. The reads are processed through a quality control program before coding regions are identified. Finally, protein sequences are compared to a database to produce quantified function profiles. The transcript abundance will also be used to identify gene expression profiles. Analysis of the genes and their expression levels are included in a report generated such as the report generated in FIG. 7.

### Example 9: Sample Preservation

In this Example, different shipment conditions were tested for their effects on preservation of nucleic acids in a sample. Soil samples were collected in three agricultural fields and shipped under different conditions. Following shipment, samples underwent shotgun metagenomic analysis. The following conditions were tested: 1) frozen at −80° C. and shipped on dry ice; 2) refrigerated and shipped on ice packs; 3) stored and shipped at ambient room temperature (i.e., traditional soil fertility); 4) stored and shipped in a nucleic acid neutralization buffer at ambient room temperature. In each field, a composite bulk soil sample was collected using a standard-sized soil probe (sanitized between samples). Samples were transported to the laboratory within 2 hours on ice. Composite samples were sieved (2-mm) and each laboratory sample, from each field, was divided into 3 subsamples. For each condition, 1 composite sample from each of 3 fields yielded 9 subsamples total. On day three (3) following sample collection, DNA was extracted and processed for library prep and next generation sequencing (NGS). Following sequencing analysis, ordination plots were generated using Bray Curtis Dissimilarity between samples to evaluate effect of 1) storage conditions and 2) location. Linear discrimination analysis Effect Size (LEfSe) was performed to determine the features (taxa, genes) most likely to explain any differences between 1) storage and 2) location.

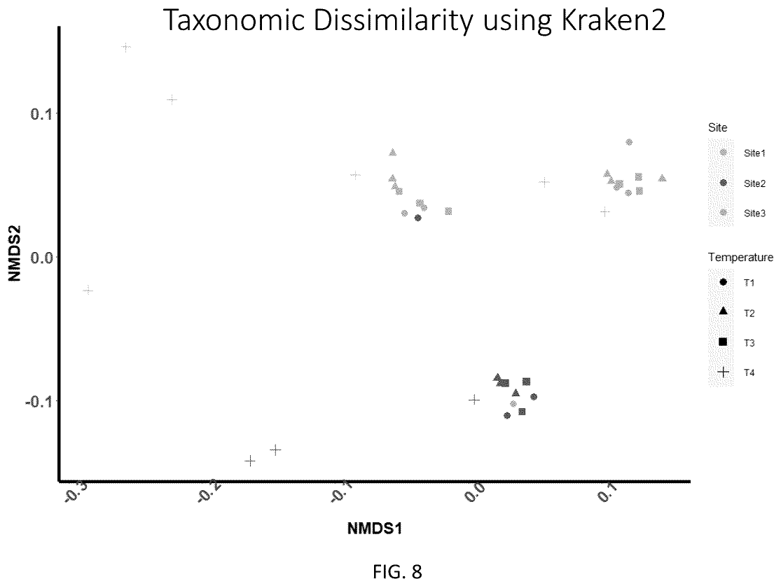

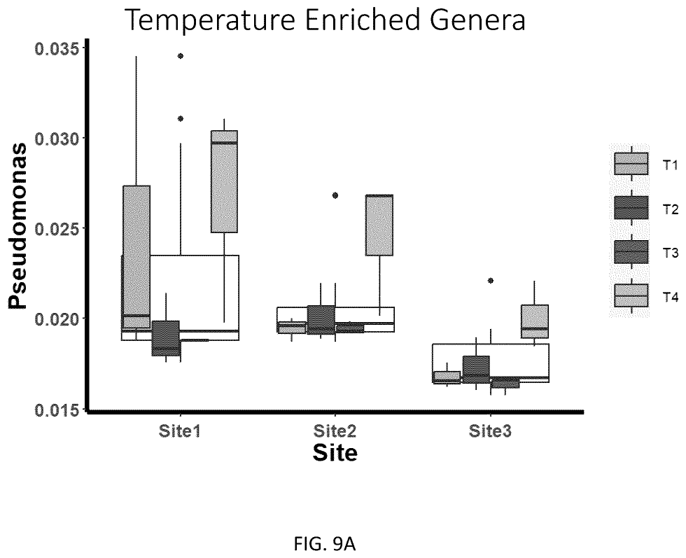

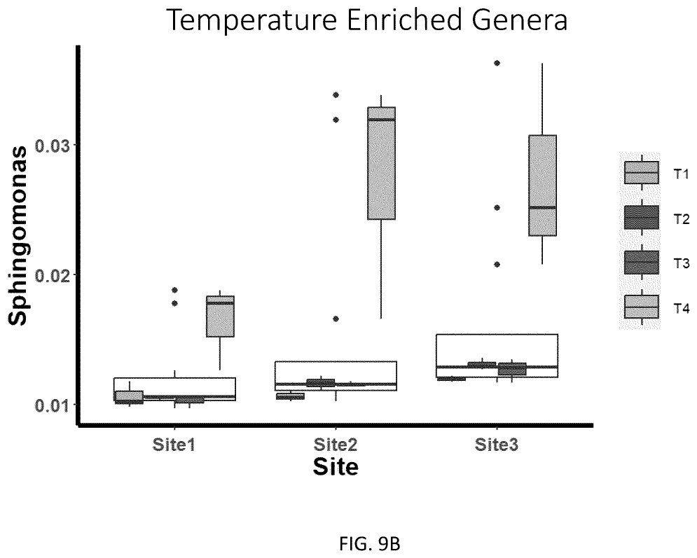

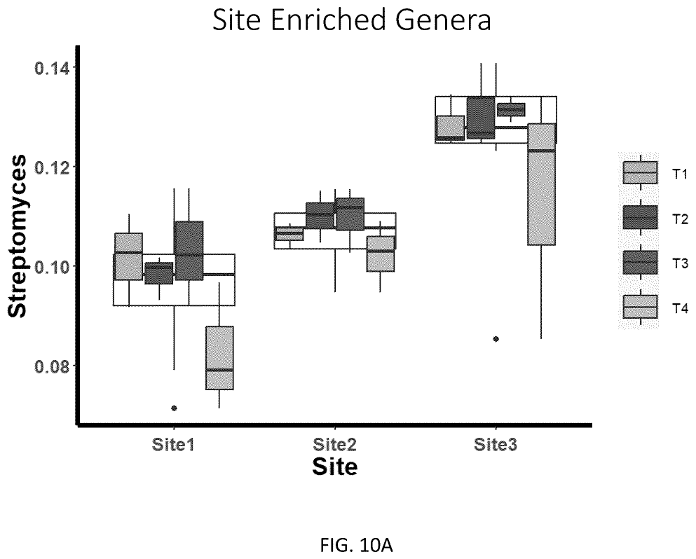

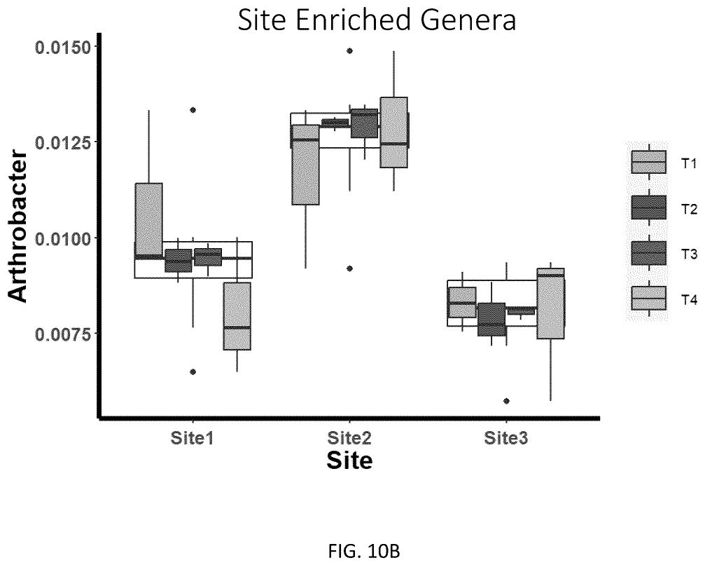

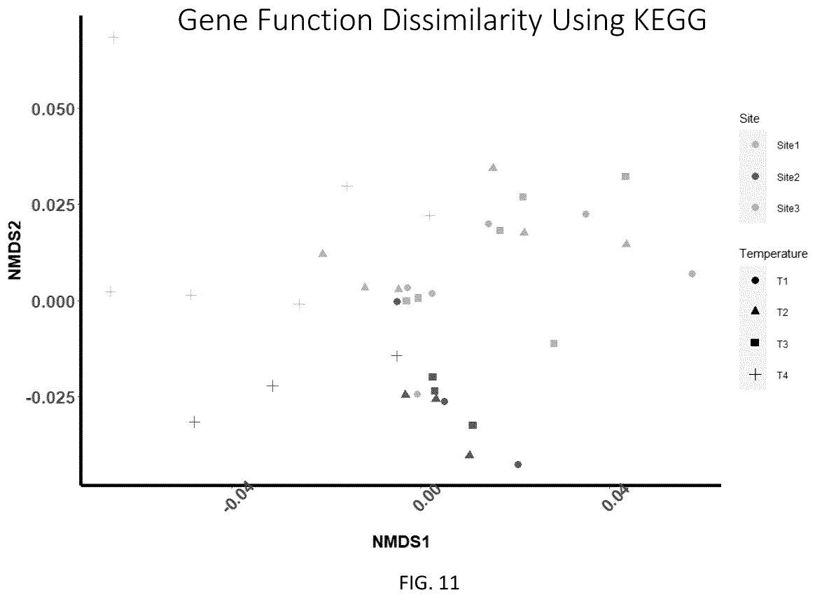

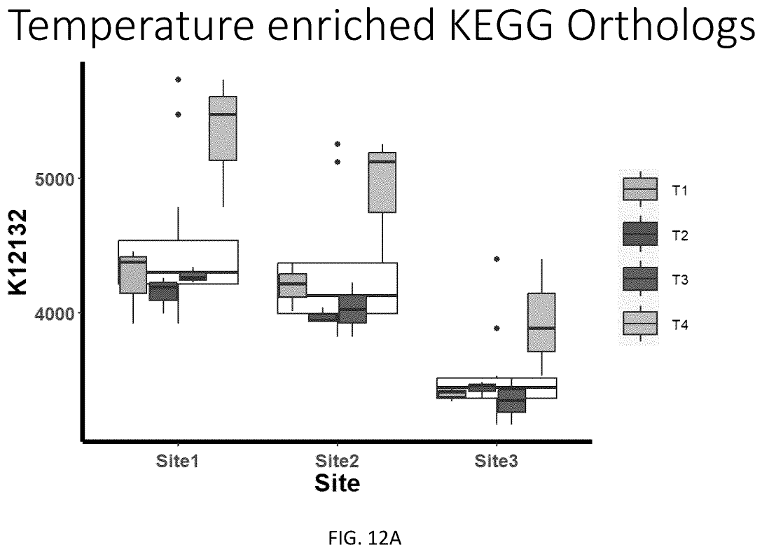

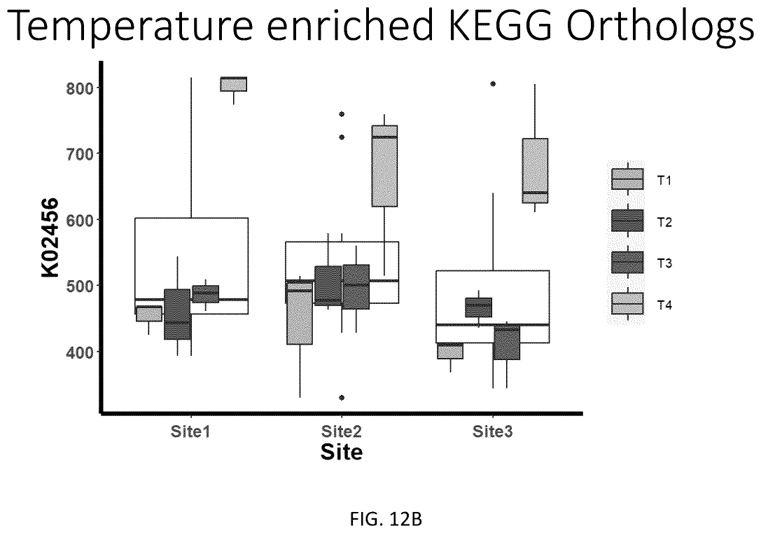

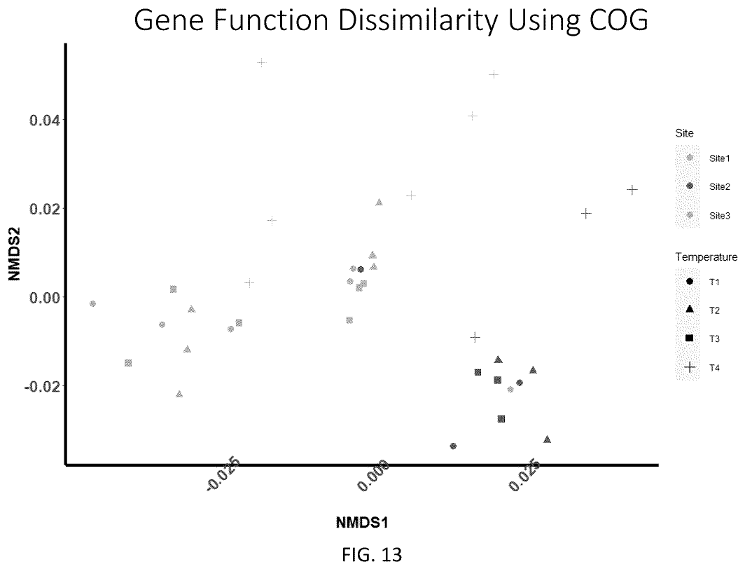

Kraken2/Bracken using PlusPF (conservative) was used to assign taxonomic labels to metagenomic DNA sequences. Kyoto Encyclopedia of Genes and Genomes (KEGG) is a collection of databases used here to evaluate DNA sequencing data to predict gene function (fine grained; 16,000 orthologues). COG (Orthologous Groups of proteins) is a database that allows for the phylogenetic classification of proteins and was used here as a second method to predict gene function (coarse grained; 5,000 orthologs). The taxonomic dissimilarity discovered using Kraken2 is shown in FIG. 8. Temperature enriched genera uncovered using this approach, in particular Pseudomonas and Sphingomonas, are shown in FIG. 9A-FIG. 9B. Site enriched genera uncovered using this approach, in particular Streptomyces and Arthrobacter, are shown in FIG. 10A-FIG. 10B. Gene function dissimilarity using KEGG is shown in FIG. 11. Temperature enriched KEGG orthologs (K12132 and K02456) uncovered using this approach are shown in FIG. 12A-FIG. 12B. K12132 corresponds to serine/threonine protein kinase (prkC and stkP) genes. K02456 corresponds to general secretion protein pathway G (gspG). Data shown in FIG. 12A-B demonstrated that the temperature of sample transport impacts gene abundance. This change may be related to the neutralization buffer used. Gene function dissimilarity using COG uncovered using this approach is shown in FIG. 13.

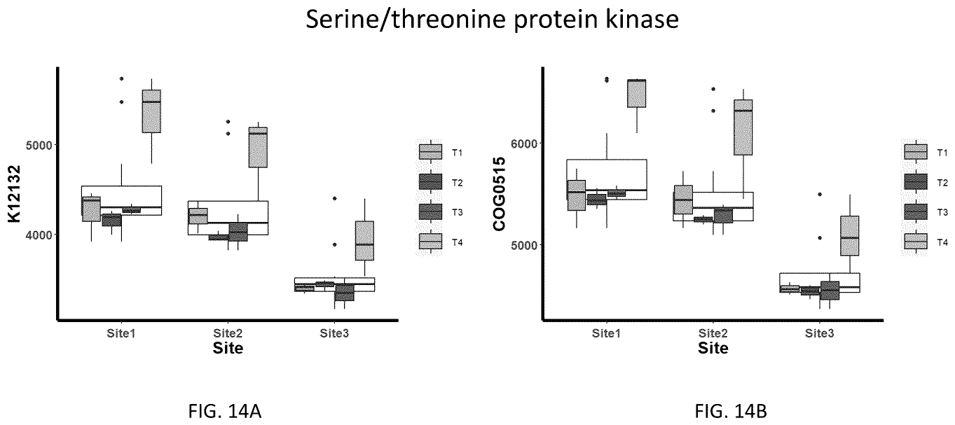

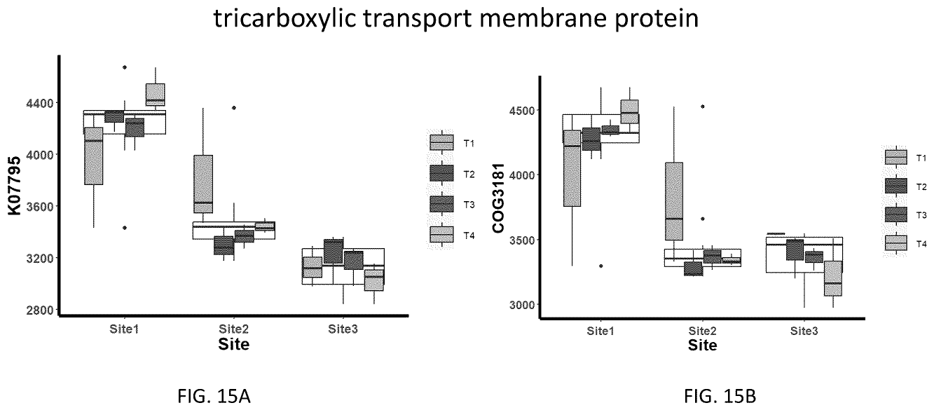

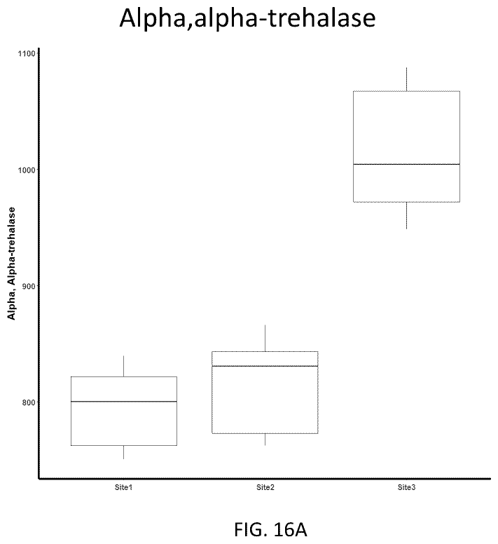

Using this approach, it was found that enriched genes are consistent between methods as shown in FIG. 14A-FIG. 14B (serine/threonine protein kinase) and FIG. 15A-FIG. 15B (tricarboxylic transport membrane protein). COG0515 corresponds to serine/threonine protein kinase genes prkC and stkP and thus corresponds to the same genes as K12132, indicating equivalence between use of the KEGG database and the COGs database. K07795 corresponds to tricarboxylic transport membrane protein (tctC). COG3181 corresponds to tricarboxylic transporter (tctC), equivalent to K07795, again demonstrating equivalence between use of the two databases as applied herein.

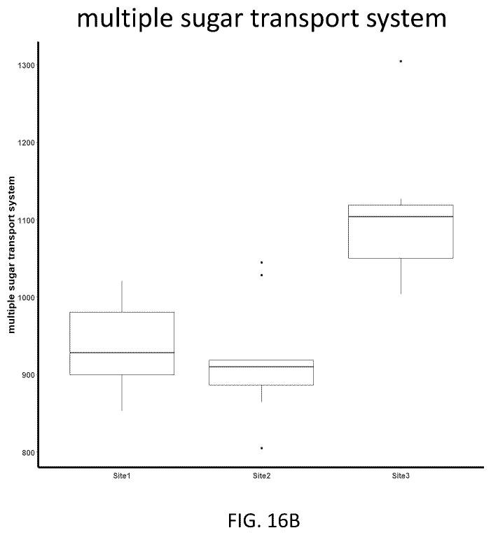

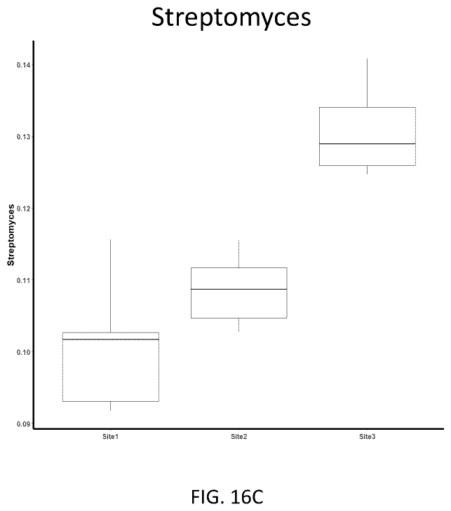

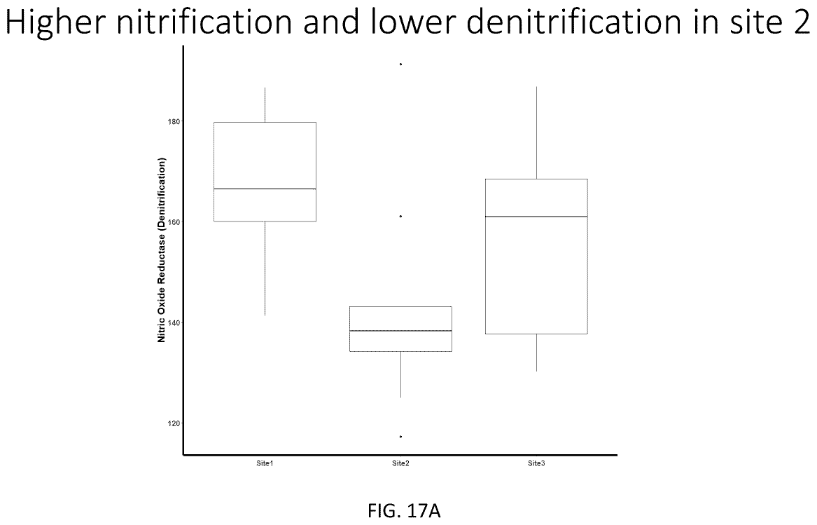

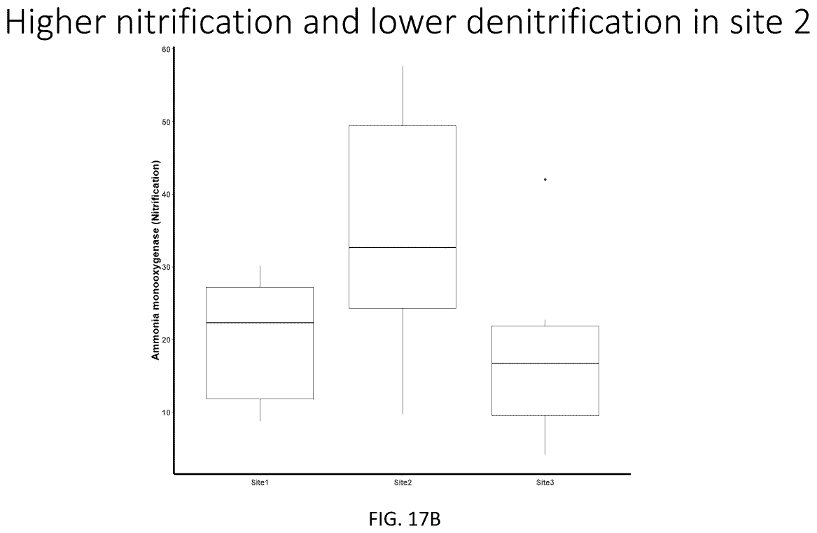

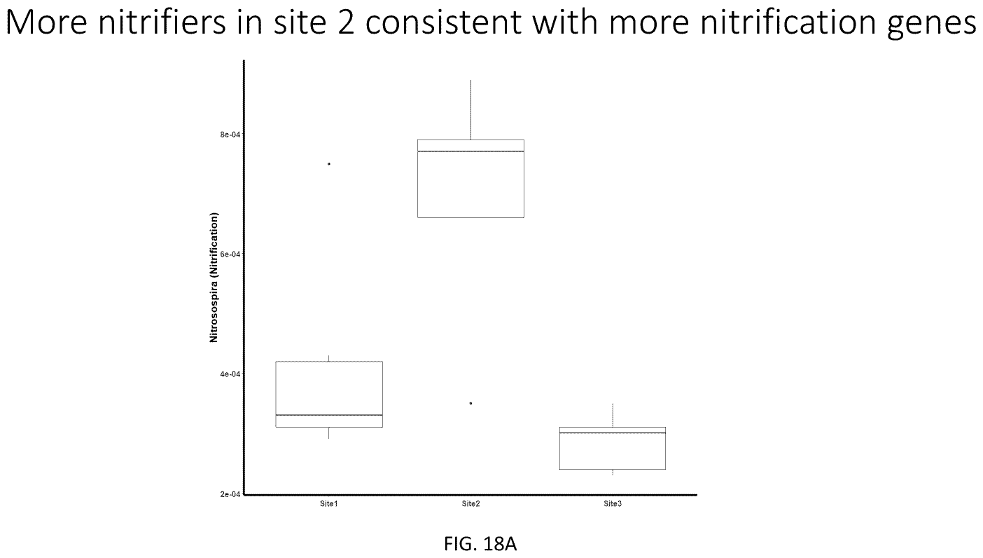

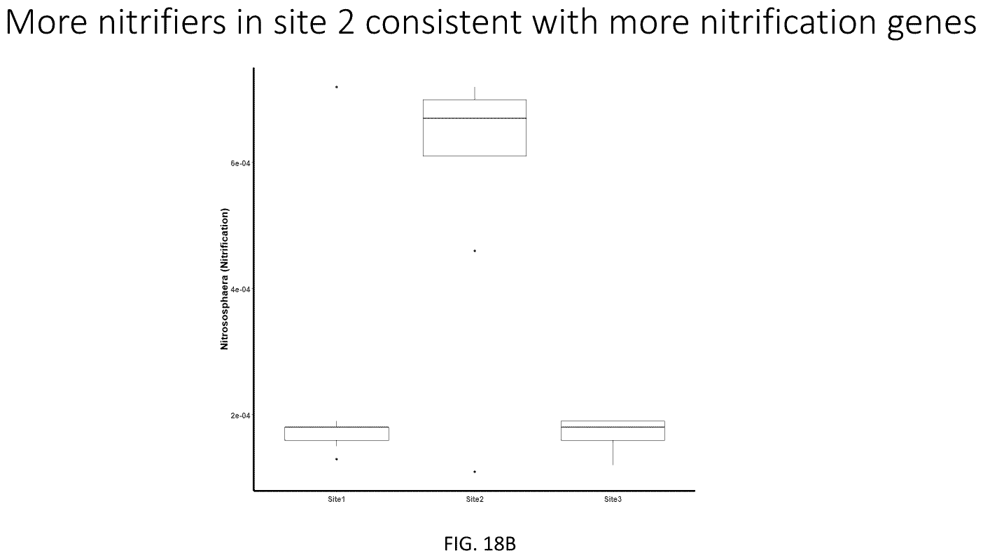

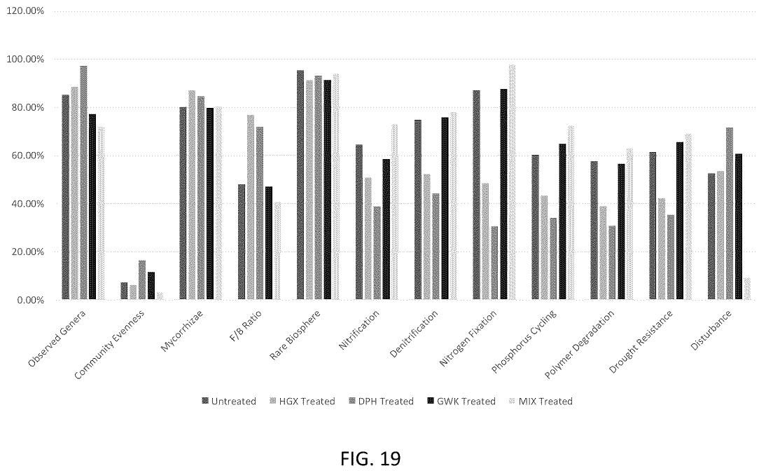

It was found that Site 3 contains higher numbers of PGP bacteria and genes as shown in FIG. 16A-FIG. 16C. FIG. 16A shows data for alpha, alpha-trehalase; FIG. 16B shows data for multiple sugar transport system genes; and FIG. 16C shows data for Streptomyces. It was found that Site 2 has reduced nitric oxide reductase (denitrification) and increased ammonia monooxygenase (nitrification) as shown in FIG. 17A-FIG. 17B. It was also found that more nitrifiers are present in Site 2, with enrichment of both Nitrosospira and Nitrososphaera, consistent with the finding of FIG. 17A-FIG. 17B, as shown in FIG. 18A-FIG. 18B.

In summary, it was found that all four analytical methods have consistent results. Site is the biggest factor identified. Using T4 (nucleic acid neutralization buffer) resulted in significantly different results from using T1, T2, or T3. It was found that enriched genes are consistent between COG and KEGG. These results indicate that the use of cold packs of room temperature storage of moist field soil within 72 hours of collection is adequate for purposes of the analysis disclosed herein.

### Example 10: Rhizosphere Analysis Report

The results in a report as shown in FIG. 19 were generated using shotgun metagenomics. The relevant genes were normalized for read depth and counted. Community members, soil function ratings, and soil risk ratings were calculated by comparing the relative abundance of species or genes in the sample to a large data set of other agricultural soils to generate a percentile, which represents the value in anormal distribution that has a specific percentage of observations below it. Results were binned into quintiles to generate ratings (i.e., 0-20% rated as very low, 20-40% rated as low, 40-60% rated as medium, 60-80% rated as high, and 80-100% rated as very high). The data shown in FIG. 19 are summarized in Table 33 below.

TABLE 33
Rhizosphere analysis report

| | | Bio- | Bio- | Bio- | Bio- |
|---|---|---|---|---|---|
| | | logical | logical | logical | logical |
| | Untreated | #1 | #2 | #3 | #4 |
| Observed Genera | 85.17% | 88.56% | 97.25% | 77.12% | 72.03% |
| Community Evenness | 7.2% | 6.14% | 16.31% | 11.44% | 3.18% |
| Mycorrhizae | 80.03% | 87.02% | 84.53% | 79.7% | 80.39% |
| F/B Ratio | 47.88% | 76.91% | 71.82% | 47.03% | 40.68% |
| Rare Biosphere | 95.34% | 91.31% | 93.22% | 91.31% | 94.07% |
| Nitrification | 64.51% | 50.81% | 38.8% | 58.47% | 73.10% |
| Denitrification | 74.82% | 52.3% | 44.11% | 75.73% | 78.13% |
| Nitrogen Fixation | 87.02% | 48.47% | 30.53% | 87.6% | 97.9% |
| Phosphorus Cycling | 60.29% | 43.28% | 34.03% | 64.77% | 72.52% |
| Polymer Degradation | 57.61% | 38.87% | 30.7% | 56.48% | 63.08% |
| Drought Resistance | 61.46% | 42.2% | 35.26% | 65.51% | 69.17% |
| Disturbance | 52.51% | 53.47% | 71.62% | 60.62% | 9.27% |
| Total Score | 4.3 | 3.5 | 3.2 | 4.3 | 4.5 |

### Example 11: Biological Input Metagenomic Report

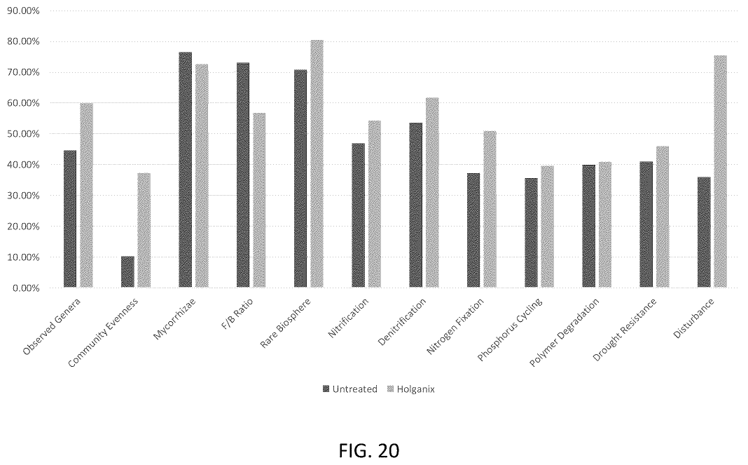

The results in this report as shown in FIG. 20 were generated using shotgun metagenomics. Soil samples were collected from a field. A rhizosphere report as described herein was generated and a beneficial plant management technique was recommended. The beneficial plant management technique recommended comprised a commercial biological soil input. Soil samples a farmer field and sent, along with farmer seed, to the laboratory for testing. The laboratory created treated and untreated rhizospheres, specific for the farmer soil, by planting the farmer seed in the farmer soil that was either treated with the commercial biological fertilizer input or was not treated with the commercial biological fertilizer input. After 7-10 days of growth in a greenhouse that simulates spring-like environmental conditions, the treated and untreated plants were collected and the DNA from the rhizosphere was collected for shotgun metagenomics. The relevant genes were normalized for read depth and counted. Community members, soil function ratings, and soil risk ratings were calculated by comparing the relative abundance of species or genes in the sample to a large data set of other agricultural soils to generate a percentile, which represents the value in a normal distribution that has a specific percentage of observations below it. Results were binned into quintiles to generate ratings (i.e., 0-20% rated as very low, 20-40% rated as low, 40-60% rated as medium, 60-80% rated as high, and 80-100% rated as very high). The data shown in FIG. 20 is summarized in Table 34 below. Implementation of the beneficial plant management technique using the commercially available biological fertilizer input resulted in an increase in community evenness, observed genera, observance of rare species, nitrogen fixing species, phosphorous cycling species, denitrification species, nitrification species, polymer degradation species, drought resistance species, and total score. The farmer used this information to make a management decision that includes the use of a commercial biological fertilizer input.

TABLE 34
Biological input metagenomic report

| | Untreated | Biological Input |
|---|---|---|
| Observed Genera | 44.55% | 59.85% |
| Community Evenness | 10.17% | 37.29% |
| Mycorrhizae | 76.52% | 72.65% |
| F/B Ratio | 73.09% | 56.78% |
| Rare Biosphere | 70.76% | 80.51% |
| Nitrification | 46.85% | 54.28% |
| Denitrification | 53.54% | 61.77% |
| Nitrogen Fixation | 37.21% | 50.95% |
| Phosphorus Cycling | 35.68% | 39.57% |
| Polymer Degradation | 39.93% | 40.92% |
| Drought Resistance | 41.00% | 45.98% |
| Disturbance | 35.93% | 75.48% |
| Total Score | 2.8 | 3.4 |

### Example 12: Exploring the Soil Microbiome Profiles of Agricultural Fields Under Different Management Techniques

The purpose of this study was to compare the soil microbiome profiles in fields with and without conventional chemical fertilizer input, as well as fields with and without tillage. By investigating these factors, valuable insights was gained into the impact of agricultural practices on soil health and sustainability. Soil microbiome analysis was performed in four fields under different management practices to determine the impact on soil microbial communities. The following fields, each under different management, were analyzed: 1. Chemical fertilizer input with tillage 2. Chemical fertilizer input without tillage 3. No chemical fertilizer input with tillage 4. No chemical fertilizer input without tillage This analysis uses whole-genome shotgun metagenomics. Shotgun metagenomics is a technique that can be used to evaluate functional changes to a microbial community. It can allow researchers to comprehensively sample all genes in all organisms present in each complex sample. This approach can afford a unique snapshot of the resident microbial community, along with the ability to compare taxonomic and functional composition across different sampling sites. Profiling the soil microbiome under different management practices serves several crucial purposes: understanding how different management practices affect soil microbiome helps to identify practices that improve soil health and functioning.

Sample collection was carried out at 15 locations on a field block which were selected for bulk soil composite sample collection. Samples were collected at a depth of 6 inches (15 cm) and mixed to create a homogenized representation of the block prior to and after tilling/chemical addition. Once 0.75 gallons of soil was collected from all locations representing all four categories, the soil was bagged and sent to the laboratory. Upon arrival at the lab, one gram of soil from each category was collected in triplicates that represent each condition. The bulk soil samples undergo DNA extraction and isolation using the DNEasy PowerSoil Pro Kit (QIAGEN, Hilden, DE) according to the manufacturer's instructions. DNA concentrations were quantified on a Qubit fluorometer via Qubit's dsDNA HS Assay Kit (Thermo Fisher Scientific, Waltham, MA).

The isolated genomic DNA was fragmented using a proportional amount of fragmentation enzyme from the Nextera XT Library Preparation kit (Illumina, Inc., San Diego, CA). Unique dual indexes (IDT Corp., Newark, NJ) were added to each individual sample followed by 12 cycles of PCR to construct libraries. DNA libraries were purified using AMpure magnetic Beads (Beckman Coulter, Brea, CA) and eluted in QIAGEN EB buffer. The DNA libraries were quantified using a Qubit fluorometer via Qubit's dsDNA HS Assay Kit. The libraries were sequenced on an Illumina NovaSeq platform, generating 2×150 bp reads. The sequence reads were passed through RhizeBio's Discovery Bioinformatics Pipeline via Microsoft Azure virtual machines. Briefly, the microbial community information contained in these reads is analyzed for function and taxonomy through the following programs: Kraken2: Generates taxonomic profiles (Wood et al., 2019); Clusters of Orthologous Genes: Generates distinct functional profiles (Tatusov et al., 2003); and Kyoto Encyclopedia of Genes and Genomes: Generates distinct functional profiles (Kanehisa et al., 2010).

Conclusions: Chemical addition combined with disc tillage generated unique communities. Chemical addition combined with disc tillage increased denitrification genes and nitrogen fixation in a way that chemical addition without tillage does not. Nitrogen fixation genes increased in the same pattern. Organic nitrogen breakdown capacity was greater without tillage.

### Example 13: Differences in Bulk Soil and Rhizosphere Soil

A trial was completed at a farm to evaluate on-farm and greenhouse performance of technology disclosed herein in capturing corn rhizomicrobiomes. Composited soil samples from different locations across a 10-acre field were collected and sent for greenhouse analysis. Corn seeds were planted in the field and collected in-field plants after 7 days. The inventors replicated this trial with the same soil and seed in a greenhouse. The inventors compared the microbiomes between planted (rhizomicrobiome) and unplanted (bulk) soils using shotgun metagenomics and the bioinformatics pipeline disclosed herein.

This work demonstrated the capacity to identify the development of early rhizomicrobiome communities. Table 35 below shows changes in selected taxonomic and genetic functional parameters for both the on-farm and greenhouse rhizosphere soils verses bulk field soils. Burkholderia were significantly enriched in rhizosphere soils while Streptococcus, Citromicrobium, and Plasmodium were significantly reduced. Burkholderia are commonly indicators of rhizosphere formation and known for their capacity to fix nitrogen and colonize root tissues. On the functional level, genes associated with nitrification (amoA and hao) are decreased in the rhizosphere soils relative to bulk soils while genes associated with nitrogen fixation (nifH) and nitrogen mineralization (cynS and ureC) are increased. These results provide evidence that technology disclosed herein can distinguish taxonomic and functional changes in the rhizomicrobiome in greenhouse and on-farm settings.

TABLE 35
Changes to relative abundance of key taxa and genetic
function between samples of bulk soil and samples of
the rhizosphere using the device disclosed herein

| | No Seed | Seed | % Change |
|---|---|---|---|
| Taxonomy* | | | |
| Burkholderia | 0.01007 | 0.218808 | 1768% |
| Streptococcus | 0.00007 | 0.000045 | −36% |
| Plasmodium | 0.00006 | 0.000035 | −42% |
| Citromicrobium | 0.00012 | 0.00006 | −50% |
| Gene Function** | | | |
| cynS (nitrogen mineralization) | 42.171 | 100.08135 | 137% |
| nifH (nitrogen fixation) | 3.0958 | 7.04012 | 127% |
| ureC (nitrogen mineralization) | 114.51 | 164.4285 | 44% |
| Hao (nitrification pathway) | 13.253 | 11.29395 | −15% |
| amoA (nitrification pathway) | 26.3439 | 6.780685 | −74% |

*On farm and greenhouse results are averaged for each condition and displayed. Units represent fraction of total community.
**On farm and greenhouse results are averaged for each condition and displayed. Units represent reads per kilobase million which are values normalized for sequencing depth and gene size in kb

### Example 14: Biological Input Increases Crop Yield

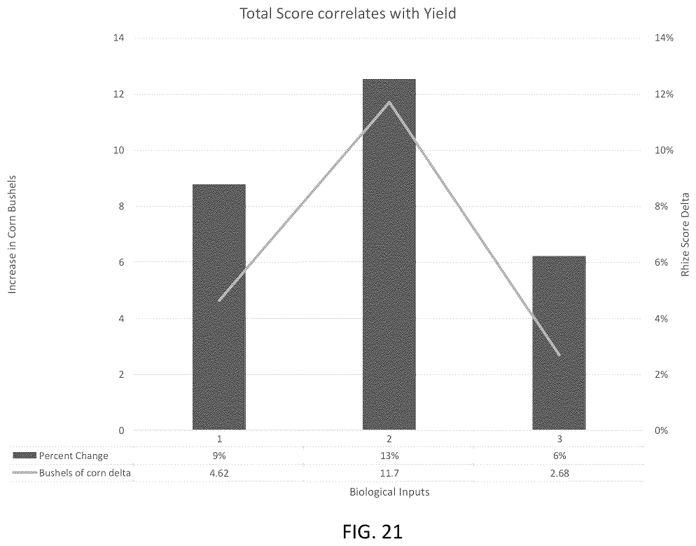

In this Example, 3 different biological inputs were tested as beneficial plant management techniques for a corn farm. Soil was collected in the middle of a growing season. Biological inputs were tested in a greenhouse setting as well as in a field setting concurrently. Results are shown in FIG. 21 and summarized in Table 36 below.

FIG. 21 shows that the Total Score calculated as shown in Table 36 correlates with crop yield. This data shows that analyzing the metagenomics of early forming rhizomicrobiomes is able to successfully predict crop yield changes due to biological input treatment. Early forming rhizosphere analysis, even without treatment, collected from a field or a greenhouse, is a critical sample type in agriculture that may be used to predict crop health, including yield estimates.

TABLE 36
Effect of biological inputs on corn yield

| | Bio- | Bio- | Bio- | |
|---|---|---|---|---|
| | logical 1 | logical 2 | logical 3 | Control |
| Observed Genera | 62.39% | 35.17% | 39.41% | 23.09% |
| Community Evenness | 4.66% | 9.53% | 4.66% | 0.85% |
| Mycorrhizae | 63.91% | 68.53% | 69.96% | 64.04% |
| F/B Ratio | 5.93% | 21.19% | 13.77% | 1.27% |
| Rare Biosphere | 92.37% | 80.72% | 84.96% | 80.72% |
| Nitrification | 31.50% | 37.06% | 34.25% | 29.41% |
| Denitrification | 67.61% | 60.52% | 70.08% | 83.08% |
| Nitrogen Fixation | 62.79% | 71.37% | 68.89% | 88.55% |
| Phosphorus Cycling | 55.44% | 57.30% | 55.01% | 62.03% |
| Polymer Degradation | 54.26% | 55.16% | 56.35% | 58.40% |
| Drought Resistance | 61.98% | 66.90% | 60.37% | 67.84% |
| Disturbance | 77.41% | 79.54% | 77.03% | 71.62% |
| Total Score | 4.3 | 4.4 | 4.2 | 3.9 |
| Delta Total Score | 0.3 | 0.5 | 0.2 | |
| Percent Change | 9% | 13% | 6% | |
| Bushels of corn delta | 4.62 | 11.7 | 2.68 | |
| Net ROI vs untreated | $18.00 | $38.50 | $4.85 | |

### Example 15: Effect of Commercial Biological Fertilizer on Functional Gene Abundance Important for Crop Health

In this Example, a beneficial plant management technique was recommended and applied to a farm as described in Example 11. The beneficial plant management technique comprised application of a commercial biological fertilizer. The effects of the commercial biological fertilizer treatment on the relative abundance of rhizomicrobiome genes involved in different plant health pathways are shown in Table 37 below. This data shows that comparing treated and untreated samples in a greenhouse setting allows for the identification and relative quantification of genes and genera of important microbes that are recruited by biological stimulants into the rhizosphere.

TABLE 37
Gene list

| | | | Biological | Delta |
|---|---|---|---|---|
| Pathway | Broad Pathway | Untreated | Fertilizer | HG |
| EPS biosynthesis | drought resistance | 0 | 3.59014 | N/A |
| EPS biosynthesis | drought resistance | 0 | 6.13447 | N/A |
| dissimilatory nitrate | nitrogen cycling | 0 | 2.27441 | N/A |
| reduction | | | | |
| EPS biosynthesis | drought resistance | 0 | 4.39386 | N/A |
| anammox | nitrogen cycling | 0 | 1.28476 | N/A |
| EPS biosynthesis | drought resistance | 0 | 1.34575 | N/A |
| EPS biosynthesis | drought resistance | 0 | 2.79863 | N/A |
| EPS biosynthesis | drought resistance | 0 | 0.851937 | N/A |
| EPS biosynthesis | drought resistance | 1.0416 | 5.53382 | 431% |
| nitrification | nitrogen cycling | 1.14237 | 4.85535 | 325% |
| EPS biosynthesis | drought resistance | 1.51904 | 4.84221 | 219% |
| EPS biosynthesis | drought resistance | 1.32613 | 4.22727 | 219% |
| EPS biosynthesis | drought resistance | 8.26446 | 26.3443 | 219% |
| EPS biosynthesis | drought resistance | 2.4454 | 5.19675 | 113% |
| EPS biosynthesis | drought resistance | 1.2241 | 2.60135 | 113% |
| EPS biosynthesis | drought resistance | 1.08624 | 2.30838 | 113% |
| EPS biosynthesis | drought resistance | 2.34947 | 4.99287 | 113% |
| organic degradation and | nitrogen cycling | 12.0445 | 24.6114 | 104% |
| synthesis | | | | |
| trehalose biosynthesis | drought resistance | 138.269 | 256.325 | 85% |
| dissimilatory nitrate | nitrogen cycling | 19.4616 | 35.8436 | 84% |
| reduction | | | | |
| Phosphonate and | phosphorous cycling | 9.76727 | 17.2971 | 77% |
| phosphinate metabolism | | | | |
| EPS biosynthesis | drought resistance | 11.1611 | 19.2713 | 73% |
| Purine metabolism | phosphorous cycling | 224.893 | 368.497 | 64% |
| EPS biosynthesis | drought resistance | 13.4971 | 21.5122 | 59% |
| osmoprotectant | drought resistance | 4.76552 | 7.59543 | 59% |
| biosynthesis | | | | |
| Pyruvate metabolism | phosphorous cycling | 306.893 | 489.134 | 59% |
| EPS biosynthesis | drought resistance | 106.148 | 168.097 | 58% |
| EPS biosynthesis | drought resistance | 95.2455 | 142.435 | 50% |
| trehalose biosynthesis | drought resistance | 123.489 | 182.511 | 48% |
| Purine metabolism | phosphorous cycling | 196.121 | 289.711 | 48% |
| trehalose biosynthesis | drought resistance | 327.096 | 480.19 | 47% |
| dissimilatory nitrate | nitrogen cycling | 6.6883 | 9.77168 | 46% |
| reduction | | | | |
| Oxidative | phosphorous cycling | 192.6 | 279.685 | 45% |
| phosphorylation | | | | |
| Pentose phosphate | phosphorous cycling | 349.876 | 507.315 | 45% |
| pathway | | | | |
| EPS biosynthesis | drought resistance | 3.15261 | 4.46643 | 42% |
| EPS biosynthesis | drought resistance | 3.6633 | 5.18994 | 42% |
| EPS biosynthesis | drought resistance | 61.7258 | 86.1632 | 40% |
| Purine metabolism | phosphorous cycling | 277.009 | 380.312 | 37% |
| Two-component system | phosphorous cycling | 302.591 | 413.383 | 37% |
| EPS biosynthesis | drought resistance | 111.014 | 151.661 | 37% |
| denitrification | nitrogen cycling | 28.7838 | 39.3227 | 37% |
| Pyrimidine metabolism | phosphorous cycling | 222.217 | 303.579 | 37% |
| organic degradation and | nitrogen cycling | 178.846 | 243.922 | 36% |
| synthesis | | | | |
| EPS biosynthesis | drought resistance | 93.6793 | 127.072 | 36% |
| EPS biosynthesis | drought resistance | 169.962 | 230.507 | 36% |
| EPS biosynthesis | drought resistance | 25.6551 | 34.6944 | 35% |
| assimilatory nitrate | nitrogen cycling | 85.9599 | 114.757 | 34% |
| reduction | | | | |
| EPS biosynthesis | drought resistance | 4.93125 | 6.54965 | 33% |
| EPS biosynthesis | drought resistance | 259.477 | 343.904 | 33% |
| Two-component system | phosphorous cycling | 393.806 | 521.149 | 32% |
| EPS biosynthesis | drought resistance | 239.279 | 314.41 | 31% |
| Others | phosphorous cycling | 364.925 | 473.48 | 30% |
| Purine metabolism | phosphorous cycling | 412.754 | 534.409 | 29% |
| Transporters | phosphorous cycling | 624.555 | 803.333 | 29% |
| organic degradation and | nitrogen cycling | 451.543 | 577.823 | 28% |
| synthesis | | | | |
| denitrification | nitrogen cycling | 92.1813 | 117.9 | 28% |
| Organic phosphoester | phosphorous cycling | 88.1527 | 112.4 | 28% |
| hydrolysis | | | | |
| Pentose phosphate | phosphorous cycling | 419.285 | 531.915 | 27% |
| pathway | | | | |
| EPS biosynthesis | drought resistance | 26.5924 | 33.5537 | 26% |
| Two-component system | phosphorous cycling | 344.985 | 430.643 | 25% |
| Transporters | phosphorous cycling | 320.761 | 398.679 | 24% |
| denitrification | nitrogen cycling | 29.5611 | 36.1217 | 22% |
| Oxidative | phosphorous cycling | 426.228 | 518.911 | 22% |
| phosphorylation | | | | |
| Purine metabolism | phosphorous cycling | 4.8939 | 5.94287 | 21% |
| Pentose phosphate | phosphorous cycling | 376.358 | 456.884 | 21% |
| pathway | | | | |
| Pentose phosphate | phosphorous cycling | 139.489 | 169.075 | 21% |
| pathway | | | | |
| Organic phosphoester | phosphorous cycling | 185.337 | 224.281 | 21% |
| hydrolysis | | | | |
| Organic phosphoester | phosphorous cycling | 113.775 | 137.119 | 21% |
| hydrolysis | | | | |
| trehalose biosynthesis | drought resistance | 136.084 | 163.584 | 20% |
| Purine metabolism | phosphorous cycling | 377.55 | 448.859 | 19% |
| Transporters | phosphorous cycling | 307.101 | 364.381 | 19% |
| trehalose biosynthesis | drought resistance | 244.935 | 290.484 | 19% |
| Purine metabolism | phosphorous cycling | 253.865 | 300.73 | 18% |
| Phosphonate and | phosphorous cycling | 154.25 | 179.105 | 16% |
| phosphinate metabolism | | | | |
| Transporters | phosphorous cycling | 351.868 | 399.583 | 14% |
| Transporters | phosphorous cycling | 200.781 | 227.649 | 13% |
| EPS biosynthesis | drought resistance | 112.427 | 127.062 | 13% |
| Purine metabolism | phosphorous cycling | 400.952 | 448.855 | 12% |
| Purine metabolism | phosphorous cycling | 414.278 | 458.662 | 11% |
| organic degradation and | nitrogen cycling | 215.797 | 233.182 | 8% |
| synthesis | | | | |
| Pyrimidine metabolism | phosphorous cycling | 344.245 | 370.083 | 8% |
| Purine metabolism | phosphorous cycling | 378.526 | 404.833 | 7% |
| EPS biosynthesis | drought resistance | 1.10604 | 1.17524 | 6% |
| Purine metabolism | phosphorous cycling | 1.94516 | 2.06686 | 6% |
| Pyrimidine metabolism | phosphorous cycling | 2.24804 | 2.38867 | 6% |
| Purine metabolism | phosphorous cycling | 3.47926 | 3.69691 | 6% |
| EPS biosynthesis | drought resistance | 4.46467 | 4.74396 | 6% |
| Purine metabolism | phosphorous cycling | 318.397 | 338.314 | 6% |
| EPS biosynthesis | drought resistance | 3.10916 | 3.30365 | 6% |
| Organic phosphoester | phosphorous cycling | 45.4237 | 48.2651 | 6% |
| hydrolysis | | | | |
| Others | phosphorous cycling | 2.4731 | 2.6278 | 6% |
| nitrification | nitrogen cycling | 10.0678 | 10.6975 | 6% |
| Pyrimidine metabolism | phosphorous cycling | 502.6 | 532.229 | 6% |
| EPS biosynthesis | drought resistance | 239.324 | 253.048 | 6% |
| Purine metabolism | phosphorous cycling | 350.739 | 370.598 | 6% |
| trehalose biosynthesis | drought resistance | 178.866 | 188.581 | 5% |
| Purine metabolism | phosphorous cycling | 588.398 | 618.301 | 5% |
| Pyrimidine metabolism | phosphorous cycling | 488.662 | 512.82 | 5% |
| Purine metabolism | phosphorous cycling | 452.023 | 473.235 | 5% |
| EPS biosynthesis | drought resistance | 261.742 | 273.361 | 4% |
| Purine metabolism | phosphorous cycling | 404.207 | 420.963 | 4% |
| Purine metabolism | phosphorous cycling | 34.9479 | 36.3439 | 4% |
| trehalose biosynthesis | drought resistance | 405.048 | 413.972 | 2% |
| Pyrimidine metabolism | phosphorous cycling | 288.742 | 294.928 | 2% |
| Purine metabolism | phosphorous cycling | 417.449 | 423.189 | 1% |
| Pentose phosphate | phosphorous cycling | 102.539 | 103.278 | 1% |
| pathway | | | | |

The effect of Biological Fertilizer treatment on representation of various genera in soil is shown below in Table 38.

TABLE 38
Genera list

| Genera | Untreated | Holganix | Delta HG |
|---|---|---|---|
| Lactiplantibacillus | 0 | 0.00004 | N/A |
| Lacticaseibacillus | 0.00001 | 0.00128 | 12700% |
| Paenibacillus | 0.00089 | 0.00747 | 739% |
| Bacillus | 0.0004 | 0.00116 | 190% |
| Nocardiopsis | 0.00077 | 0.00208 | 170% |
| Frankia | 0.0013 | 0.00336 | 158% |
| Miltoncostaea | 0.0011 | 0.00279 | 154% |
| Baekduia | 0.00318 | 0.00802 | 152% |
| Conexibacter | 0.00476 | 0.01189 | 150% |
| Rhodoplanes | 0.00122 | 0.00304 | 149% |
| Capillimicrobium | 0.00213 | 0.00528 | 148% |
| Streptomyces | 0.03982 | 0.09868 | 148% |
| Aquisphaera | 0.00111 | 0.0027 | 143% |
| Paraconexibacter | 0.00124 | 0.003 | 142% |
| Streptosporangium | 0.0008 | 0.00193 | 141% |
| Pseudolabrys | 0.00128 | 0.00308 | 141% |
| Tautonia | 0.00077 | 0.00185 | 140% |
| Rhodopseudomonas | 0.00137 | 0.00325 | 137% |
| Stenotrophomonas | 0.00127 | 0.00301 | 137% |
| Corynebacterium | 0.00194 | 0.00457 | 136% |
| Saccharopolyspora | 0.00124 | 0.00291 | 135% |
| Actinoplanes | 0.003 | 0.00701 | 134% |
| Mycobacterium | 0.00696 | 0.01624 | 133% |
| Saccharothrix | 0.00084 | 0.00196 | 133% |
| Ralstonia | 0.01004 | 0.02338 | 133% |
| Paludisphaera | 0.0008 | 0.00186 | 133% |
| Kribbella | 0.00126 | 0.00292 | 132% |
| Amycolatopsis | 0.00552 | 0.01279 | 132% |
| Kitasatospora | 0.0013 | 0.003 | 131% |
| Anaeromyxobacter | 0.00115 | 0.00264 | 130% |
| Planctomyces | 0.00078 | 0.00179 | 129% |
| Kutzneria | 0.0011 | 0.00252 | 129% |
| Actinomadura | 0.00271 | 0.0062 | 129% |
| Gordonia | 0.00175 | 0.004 | 129% |
| Roseomonas | 0.00089 | 0.00202 | 127% |
| Micromonospora | 0.00691 | 0.01568 | 127% |
| Cellulomonas | 0.00319 | 0.00712 | 123% |
| Dactylosporangium | 0.00209 | 0.00466 | 123% |
| Pseudonocardia | 0.00393 | 0.00871 | 122% |
| Nonomuraea | 0.00183 | 0.00405 | 121% |
| Thermomonospora | 0.00088 | 0.00194 | 120% |
| Nocardia | 0.00304 | 0.00662 | 118% |
| Jatrophihabitans | 0.00098 | 0.00211 | 115% |
| Bradyrhizobium | 0.04487 | 0.09526 | 112% |
| Sorangium | 0.00115 | 0.00244 | 112% |
| Microlunatus | 0.00105 | 0.00222 | 111% |
| Aeromicrobium | 0.00128 | 0.00266 | 108% |
| Rathayibacter | 0.0007 | 0.00142 | 103% |
| Nocardioides | 0.01718 | 0.03468 | 102% |
| Bosea | 0.00162 | 0.00325 | 101% |
| Edaphobacter | 0.00055 | 0.00109 | 98% |
| Methylobacterium | 0.0032 | 0.00634 | 98% |
| Bordetella | 0.00094 | 0.00186 | 98% |
| Brevundimonas | 0.00094 | 0.00184 | 96% |
| Agromyces | 0.00135 | 0.0026 | 93% |
| Caulobacter | 0.00123 | 0.00235 | 91% |
| Curtobacterium | 0.00101 | 0.0019 | 88% |
| Mycolicibacterium | 0.00964 | 0.01771 | 84% |
| Xanthomonas | 0.00152 | 0.00261 | 72% |
| Paracoccus | 0.00127 | 0.00218 | 72% |
| Lysobacter | 0.00198 | 0.00334 | 69% |
| Rhodococcus | 0.00526 | 0.00885 | 68% |
| Acidovorax | 0.00198 | 0.00324 | 64% |
| Comamonas | 0.00122 | 0.00198 | 62% |
| Sphingopyxis | 0.00122 | 0.00189 | 55% |
| Mesorhizobium | 0.01021 | 0.0153 | 50% |
| Rhizobium | 0.00902 | 0.01324 | 47% |
| Achromobacter | 0.0037 | 0.00528 | 43% |
| Sphingobium | 0.00246 | 0.00342 | 39% |
| Agrobacterium | 0.00134 | 0.00178 | 33% |
| Paenarthrobacter | 0.00423 | 0.0055 | 30% |
| Pseudarthrobacter | 0.00288 | 0.00353 | 23% |
| Luteibacter | 0.0009 | 0.00108 | 20% |
| Microbacterium | 0.01446 | 0.01728 | 20% |
| Homo | 0.00291 | 0.00335 | 15% |
| Arthrobacter | 0.02118 | 0.02299 | 9% |
| Variovorax | 0.00634 | 0.00684 | 8% |

### Example 16: Manure Metagenomic Report

In this Example, metagenomic analyses of the present disclosure were carried out on samples of manure obtained from a company that treats swine manure lagoons to get the manure ready for fertilizer on farms. Three manure pits (lagoons) were sampled for analysis. The analyses screened for pathogens and provided detailed information on oxygen metabolism characteristics (i.e., aerobe, anaerobe, etc.) of the sample. The report generated described the taxonomy of each sample by estimating the percentage of each genera (top 100 genera) and categorized each genera according to its oxygen metabolism.

Table 39 below shows the results of metagenomic analysis of manure samples.

TABLE 39
Manure metagenomic test report

| | Manure Pit or Lagoon Name | | | | | |
|---|---|---|---|---|---|---|
| | Pit/Lagoon #1 | | Pit/Lagoon #2 | | Pit/Lagoon #3 | |
| | Number of Microbes, Viruses or Pathogens | | | | | |
| | 1,908 | | 1,846 | | 1,764 | |
| Top 10 Percentage | 49% | | 38% | | 40% | |
| Top 50 Percentage | 73% | | 63% | | 69% | |
| Top 100 | 81% | | 73% | | 77% | |
| Percentage | | | | | | |
| Virus Percentage - | 0 0.0000% | | 0 0.0000% | | 0 0.0000% | |
| Top 100 | | | | | | |
| Pathogens | 0 0.0000% | | 0 0.0000% | | 0 0.0000% | |
| Percentage - Top 100 | | | | | | |
| Bacteria to Fungal | 387 to 1 | | 250 to 1 | | 298 to 1 | |
| (B/F) Ratio | | | | | | |
| Fungus | | | | | | |
| Fungal Species | 48 | 0.13% | 44 | 0.19% | 42 | 0.16% |
| Number & | | | | | | |
| Percentage | | | | | | |
| Anaerobic Fungal | | | | | | |
| Species and | | | | | | |
| Percentage | 4 | 0.0800% | 4 | 0.1300% | 4 | 0.1100% |
| Aerobic Fungal | | | | | | |
| Species and | | | | | | |
| Percentage | 35 | 0.09% | 32 | 0.13% | 30 | 0.11% |
| Bacteria | | | | | | |
| Anaerobic Bacteria | 265 | 35% | 260 | 35% | 258 | 37% |
| Species and | | | | | | |
| Percentage | | | | | | |
| Aerobic Bacterial | 946 | 50% | 926 | 60% | 898 | 63% |
| Species and | | | | | | |
| Percentage | | | | | | |
| Facultative | 119 | 12% | 116 | 9% | 113 | 7% |
| Anaerobic Organisms | | | | | | |
| Obligate Anaerobic | 19 | 0.95% | 18 | 0.91% | 18 | 1.26% |
| Organisms | | | | | | |
| Microaerophilic | 56 | 17% | 57 | 9% | 52 | 7% |
| Organisms | | | | | | |
| Ectomycorrhizal to | .82 to 1 | | .92 to 1 | | 1.37 to 1 | |
| Arbuscular Ratio | | | | | | |
| Crop Functions of | | | | | | |
| Interest | | | | | | |
| Anoxic Environment | 38% | Low | 46% | Medium | 47% | Medium |
| High Oxygen | 30% | Low | 31% | Low | 30% | Low |
| Environment | | | | | | |
| Low Oxygen | 2% | Very Low | 14% | Very Low | 15% | Very Low |
| Environment | | | | | | |
| Carbon Fixation | 58% | Medium | 43% | Medium | 66% | High |
| Organic Carbon | 64% | High | 56% | Medium | 55% | Medium |
| Breakdown | | | | | | |
| Methanogenesis | 85% | Very High | 74% | High | 86% | Very High |
| Denitrification | 8% | Very Low | 23% | Low | 7% | Very Low |
| Nitrification | 8% | Very Low | 21% | Low | 0% | Very Low |
| Nitrogen Fixation | 93% | Very High | 84% | Very High | 84% | Very High |
| Organic Nitrogen | 62% | High | 67% | High | 63% | High |
| Breakdown | | | | | | |
| Phosphorus | 25% | Low | 29% | Low | 35% | Low |
| Mobilization | | | | | | |
| Potassium | 60% | High | 60% | High | 60% | High |
| Solubilization | | | | | | |
| Sulfur Oxidation | 7% | Very Low | 18% | Very Low | 15% | Very Low |
| Sulfur Reduction | 66% | High | 47% | Medium | 55% | Medium |
| Calcium Transport | 67% | High | 67% | High | 66% | High |
| Iron Acquisition | 49% | Medium | 51% | Medium | 56% | Medium |
| Plant Stress | 46% | Medium | 41% | Medium | 40% | Medium |
| Adaptation | | | | | | |

Table 40 below shows the breakdown of taxonomy uncovered in lagoon #1. The percentage of each genera for the top 100 genera is estimated, and each genera is categorized according to its oxygen metabolism.

TABLE 40
Genera in lagoon #1

| Genus | Percentage | Classification |
|---|---|---|
| Lactobacillus | 14.39% | microaerophilic |
| Streptococcus | 9.97% | facultative |
| Limosilactobacillus | 4.81% | |
| Clostridium | 4.03% | anaerobic |
| Prevotella | 3.89% | anaerobic |
| Blautia | 2.86% | anaerobic |
| Methanoculleus | 2.78% | anaerobic |
| Faecalibacterium | 2.56% | anaerobic |
| Bacteroides | 2.10% | anaerobic |
| Coprococcus | 1.81% | anaerobic |
| Corynebacterium | 1.64% | aerobic |
| Phascolarctobacterium | 1.50% | anaerobic |
| Turicibacter | 1.19% | anaerobic |
| Denitrificimonas | 1.17% | |
| Pseudomonas | 1.15% | aerobic |
| Sphaerochaeta | 0.92% | anaerobic |
| Alkaliflexus | 0.91% | anaerobic |
| Mediterraneibacter | 0.86% | |
| Enterococcus | 0.80% | microaerophilic |
| Catenibacterium | 0.77% | anaerobic |
| Staphylococcus | 0.77% | aerobic |
| Roseburia | 0.77% | anaerobic |
| Jeotgalibaca | 0.76% | microaerophilic |
| Paenibacillus | 0.75% | aerobic |
| Anaerobutyricum | 0.63% | |
| Dorea | 0.61% | anaerobic |
| Bacillus | 0.59% | aerobic |
| Romboutsia | 0.59% | anaerobic |
| Megasphaera | 0.51% | anaerobic |
| Terrisporobacter | 0.49% | anaerobic |
| Flavobacterium | 0.47% | aerobic |
| Vescimonas | 0.43% | |
| Aerococcus | 0.41% | aerobic |
| Clostridioides | 0.37% | anaerobic |
| Collinsella | 0.37% | anaerobic |
| Subdoligranulum | 0.36% | anaerobic |
| Ligilactobacillus | 0.34% | |
| Paenalcaligenes | 0.34% | facultative |
| Methanogenium | 0.32% | anaerobic |
| Ruminococcus | 0.31% | anaerobic |
| Proteiniphilum | 0.31% | obligate_anaerobic |
| Schnuerera | 0.30% | |
| Phocaeicola | 0.30% | obligate_anaerobic |
| Acidilutibacter | 0.29% | |
| Parabacteroides | 0.29% | anaerobic |
| Escherichia | 0.29% | facultative |
| Streptomyces | 0.28% | aerobic |
| Eubacterium | 0.27% | anaerobic |
| Treponema | 0.27% | anaerobic |
| Dialister | 0.27% | anaerobic |
| Lachnoclostridium | 0.26% | anaerobic |
| Acidaminococcus | 0.26% | anaerobic |
| Vagococcus | 0.26% | microaerophilic |
| Erysipelothrix | 0.25% | microaerophilic |
| Herbinix | 0.25% | anaerobic |
| Anaerocolumna | 0.25% | anaerobic |
| Chryseobacterium | 0.25% | aerobic |
| Dysosmobacter | 0.24% | |
| Butyrivibrio | 0.24% | anaerobic |
| Thiopseudomonas | 0.23% | facultative |
| Vibrio | 0.23% | facultative |
| Desulfovibrio | 0.22% | anaerobic |
| Acinetobacter | 0.21% | aerobic |
| Salmonella | 0.19% | facultative |
| Campylobacter | 0.18% | microaerophilic |
| Weissella | 0.17% | aerobic |
| Selenomonas | 0.17% | anaerobic |
| Alkalitalea | 0.17% | anaerobic |
| Simiaoa | 0.17% | |
| Enterocloster | 0.17% | |
| Acholeplasma | 0.16% | facultative |
| Lysinibacillus | 0.15% | obligate_aerobic |
| Alistipes | 0.15% | anaerobic |
| Petrimonas | 0.15% | anaerobic |
| Lachnospira | 0.14% | anaerobic |
| Methanobrevibacter | 0.14% | anaerobic |
| Flavonifractor | 0.14% | obligate_anaerobic |
| Fusobacterium | 0.14% | anaerobic |
| Burzaovirus | 0.14% | |
| Anaerostipes | 0.14% | anaerobic |
| Acetivibrio | 0.14% | obligate_anaerobic |
| Sphingobacterium | 0.13% | obligate_aerobic |
| Flintibacter | 0.12% | |
| Wujia | 0.12% | |
| Alkaliphilus | 0.12% | anaerobic |
| Bifidobacterium | 0.12% | anaerobic |
| Keratinibaculum | 0.12% | anaerobic |
| Haploplasma | 0.11% | |
| Oscillibacter | 0.11% | anaerobic |
| Shewanella | 0.11% | facultative |
| Methanosphaera | 0.11% | anaerobic |
| Tissierella | 0.11% | anaerobic |
| Paracholeplasma | 0.11% | |
| Lacrimispora | 0.11% | |
| Companilactobacillus | 0.11% | |
| Klebsiella | 0.11% | aerobic |
| Psychrobacter | 0.10% | aerobic |
| Novisyntrophococcus | 0.10% | |
| Lactococcus | 0.10% | facultative |
| Hungatella | 0.10% | anaerobic |

Table 41 below shows the breakdown of taxonomy uncovered in lagoon #2. The percentage of each genera for the top 100 genera is estimated, and each genera is categorized according to its oxygen metabolism.

TABLE 41
Genera in lagoon #2

| Genus | Percentage | Classification |
|---|---|---|
| Clostridium | 9.87% | anaerobic |
| Streptococcus | 5.67% | facultative |
| Lactobacillus | 5.19% | microaerophilic |
| Turicibacter | 4.04% | anaerobic |
| Denitrificimonas | 3.17% | |
| Bacteroides | 2.63% | anaerobic |
| Staphylococcus | 2.21% | aerobic |
| Limosilactobacillus | 1.89% | |
| Methanoculleus | 1.69% | anaerobic |
| Bacillus | 1.47% | aerobic |
| Blautia | 1.47% | anaerobic |
| Paenibacillus | 1.45% | aerobic |
| Corynebacterium | 1.42% | aerobic |
| Pseudomonas | 1.41% | aerobic |
| Enterococcus | 1.41% | microaerophilic |
| Romboutsia | 1.29% | anaerobic |
| Faecalibacterium | 1.08% | anaerobic |
| Flavobacterium | 0.95% | aerobic |
| Prevotella | 0.94% | anaerobic |
| Terrisporobacter | 0.92% | anaerobic |
| Coprococcus | 0.82% | anaerobic |
| Schnuerera | 0.81% | |
| Jeotgalibaca | 0.74% | microaerophilic |
| Clostridioides | 0.66% | anaerobic |
| Acidilutibacter | 0.58% | |
| Brevibacterium | 0.52% | aerobic |
| Brachybacterium | 0.49% | aerobic |
| Aerococcus | 0.48% | aerobic |
| Vagococcus | 0.47% | microaerophilic |
| Ligilactobacillus | 0.44% | |
| Anaerobutyricum | 0.43% | |
| Phascolarctobacterium | 0.41% | anaerobic |
| Streptomyces | 0.41% | aerobic |
| Keratinibaculum | 0.40% | anaerobic |
| Dietzia | 0.40% | obligate_aerobic |
| Treponema | 0.39% | anaerobic |
| Vescimonas | 0.38% | |
| Mediterraneibacter | 0.38% | |
| Roseburia | 0.37% | anaerobic |
| Psychrobacter | 0.36% | aerobic |
| Listeria | 0.35% | aerobic |
| Chryseobacterium | 0.35% | aerobic |
| Acinetobacter | 0.34% | aerobic |
| Proteiniphilum | 0.34% | obligate_anaerobic |
| Nocardiopsis | 0.34% | aerobic |
| Erysipelothrix | 0.33% | microaerophilic |
| Sphaerochaeta | 0.33% | anaerobic |
| Vibrio | 0.33% | facultative |
| Ignatzschineria | 0.33% | aerobic |
| Catenibacterium | 0.30% | anaerobic |
| Alkaliphilus | 0.29% | anaerobic |
| Collinsella | 0.29% | anaerobic |
| Acholeplasma | 0.29% | facultative |
| Methanobrevibacter | 0.29% | anaerobic |
| Dorea | 0.28% | anaerobic |
| Salmonella | 0.27% | facultative |
| Fusobacterium | 0.26% | anaerobic |
| Anaerocolumna | 0.26% | anaerobic |
| Parabacteroides | 0.26% | anaerobic |
| Lysinibacillus | 0.25% | obligate_aerobic |
| Priestia | 0.25% | |
| Virgibacillus | 0.25% | aerobic |
| Microbacterium | 0.24% | aerobic |
| Herbinix | 0.24% | anaerobic |
| Escherichia | 0.24% | facultative |
| Haploplasma | 0.23% | |
| Phocaeicola | 0.23% | obligate_anaerobic |
| Campylobacter | 0.20% | microaerophilic |
| Paenalcaligenes | 0.20% | facultative |
| Thiopseudomonas | 0.20% | facultative |
| Petrimonas | 0.20% | anaerobic |
| Jeotgalicoccus | 0.20% | aerobic |
| Tissierella | 0.20% | anaerobic |
| Sphingobacterium | 0.19% | obligate_aerobic |
| Polaribacter | 0.19% | aerobic |
| Eubacterium | 0.18% | anaerobic |
| Lactococcus | 0.18% | facultative |
| Subdoligranulum | 0.18% | anaerobic |
| Sporosarcina | 0.18% | aerobic |
| Lachnoclostridium | 0.17% | anaerobic |
| Dialister | 0.17% | anaerobic |
| Acetivibrio | 0.17% | obligate_anaerobic |
| Shewanella | 0.17% | facultative |
| Spiroplasma | 0.16% | microaerophilic |
| Ruminococcus | 0.16% | anaerobic |
| Desulfovibrio | 0.16% | anaerobic |
| Dysosmobacter | 0.16% | |
| Crassaminicella | 0.16% | |
| Carnobacterium | 0.15% | aerobic |
| Anaerococcus | 0.15% | anaerobic |
| Halomonas | 0.15% | obligate_aerobic |
| Arthrobacter | 0.14% | obligate_aerobic |
| Methanosphaera | 0.14% | anaerobic |
| Paracoccus | 0.14% | aerobic |
| Tetragenococcus | 0.14% | aerobic |
| Mammaliicoccus | 0.13% | |
| Oligella | 0.13% | aerobic |
| Mycoplasma | 0.13% | facultative |
| Klebsiella | 0.13% | aerobic |
| Pseudoalteromonas | 0.13% | aerobic |

Table 42 below shows the breakdown of taxonomy uncovered in lagoon #3. The percentage of each genera for the top 100 genera is estimated, and each genera is categorized according to its oxygen metabolism.

TABLE 42
Genera in lagoon #3

| | Per- | |
|---|---|---|
| Genus | centage | Classification |
| Clostridium | 7.80% | anaerobic |
| Bacteroides | 7.21% | anaerobic |
| Denitrificimonas | 5.75% | |
| Streptococcus | 4.24% | facultative |
| Corynebacterium | 3.33% | aerobic |
| Lactobacillus | 2.80% | microaerophilic |
| Turicibacter | 2.42% | anaerobic |
| Staphylococcus | 2.29% | aerobic |
| Psychrobacter | 2.08% | aerobic |
| Romboutsia | 2.06% | anaerobic |
| Pseudomonas | 2.04% | aerobic |
| Limosilactobacillus | 1.87% | |
| Methanoculleus | 1.57% | anaerobic |
| Coprococcus | 1.51% | anaerobic |
| Enterococcus | 1.39% | microaerophilic |
| Aerococcus | 1.34% | aerobic |
| Bacillus | 1.24% | aerobic |
| Prevotella | 1.22% | anaerobic |
| Jeotgalibaca | 1.18% | microaerophilic |
| Terrisporobacter | 1.16% | anaerobic |
| Paenibacillus | 1.12% | aerobic |
| Flavobacterium | 1.05% | aerobic |
| Blautia | 0.91% | anaerobic |
| Faecalibacterium | 0.82% | anaerobic |
| Schnuerera | 0.74% | |
| Phascolarctobacterium | 0.74% | anaerobic |
| Vescimonas | 0.67% | |
| Jeotgalicoccus | 0.58% | aerobic |
| Acidilutibacter | 0.50% | |
| Phocaeicola | 0.46% | obligate_anaerobic |
| Methanobrevibacter | 0.45% | anaerobic |
| Acinetobacter | 0.43% | aerobic |
| Vagococcus | 0.42% | microaerophilic |
| Keratinibaculum | 0.41% | anaerobic |
| Parabacteroides | 0.41% | anaerobic |
| Proteiniphilum | 0.40% | obligate_anaerobic |
| Miniphocaeibacter | 0.38% | |
| Chryseobacterium | 0.37% | aerobic |
| Clostridioides | 0.37% | anaerobic |
| Roseburia | 0.37% | anaerobic |
| Vibrio | 0.34% | facultative |
| Dysosmobacter | 0.33% | |
| Latilactobacillus | 0.31% | |
| Carnobacterium | 0.29% | aerobic |
| Desulfovibrio | 0.27% | anaerobic |
| Sphingobacterium | 0.27% | obligate_aerobic |
| Streptomyces | 0.26% | aerobic |
| Erysipelothrix | 0.26% | microaerophilic |
| Candidatus_Methanomethylophilus | 0.26% | |
| Treponema | 0.26% | anaerobic |
| Thiopseudomonas | 0.25% | facultative |
| Polaribacter | 0.24% | aerobic |
| Anaerococcus | 0.24% | anaerobic |
| Ruminococcus | 0.23% | anaerobic |
| Fusobacterium | 0.22% | anaerobic |
| Flavonifractor | 0.21% | obligate_anaerobic |
| Anaerocolumna | 0.21% | anaerobic |
| Escherichia | 0.20% | facultative |
| Lysinibacillus | 0.19% | obligate_aerobic |
| Lachnoclostridium | 0.19% | anaerobic |
| Mediterraneibacter | 0.19% | |
| Tissierella | 0.19% | anaerobic |
| Herbinix | 0.18% | anaerobic |
| Eubacterium | 0.18% | anaerobic |
| Butyrivibrio | 0.18% | anaerobic |
| Campylobacter | 0.18% | microaerophilic |
| Methanosphaera | 0.18% | anaerobic |
| Shewanella | 0.18% | facultative |
| Listeria | 0.18% | aerobic |
| Anaerobutyricum | 0.17% | |
| Myroides | 0.17% | aerobic |
| Blattabacterium | 0.17% | |
| Dorea | 0.16% | anaerobic |
| Tenacibaculum | 0.16% | obligate_aerobic |
| Lactococcus | 0.16% | facultative |
| Priestia | 0.15% | |
| Enterocloster | 0.15% | |
| Trueperella | 0.15% | microaerophilic |
| Flintibacter | 0.15% | |
| Tetragenococcus | 0.15% | aerobic |
| Pseudoalteromonas | 0.14% | aerobic |
| Petrimonas | 0.14% | anaerobic |
| Sphaerochaeta | 0.14% | anaerobic |
| Paeniclostridium | 0.14% | anaerobic |
| Oscillibacter | 0.14% | anaerobic |
| Sporosarcina | 0.14% | aerobic |
| Crassaminicella | 0.13% | |
| Klebsiella | 0.13% | aerobic |
| Subdoligranulum | 0.13% | anaerobic |
| Ligilactobacillus | 0.13% | |
| Porphyromonas | 0.13% | anaerobic |
| Paraclostridium | 0.13% | anaerobic |
| Capnocytophaga | 0.13% | microaerophilic |
| Pedobacter | 0.13% | obligate_aerobic |
| Macrococcus | 0.13% | aerobic |
| Virgibacillus | 0.12% | aerobic |
| Collinsella | 0.12% | anaerobic |
| Alkaliphilus | 0.12% | anaerobic |
| Dietzia | 0.12% | obligate_aerobic |
| Pusillibacter | 0.12% | |

While some embodiments have been shown and described herein, such embodiments are provided by way of example only. Numerous variations, changes, and substitutions will now occur to those skilled in the art without departing from the disclosure provided herein. It should be understood that the order of methods disclosed herein can be rearranged, removed or modified. It should be understood that various alternatives to the embodiments described herein can be employed.

1.-78. (canceled)

79. A method for analyzing a soil sample, said method comprising:
a) germinating a seed in conditions for said seed to release exudates in said soil;
b) recruiting one or more microorganisms to said exudates to generate a rhizosphere; and
c) sequencing genetic material of said one or more microorganisms.

80. The method of claim 79, wherein said conditions comprise one or more of: soil composition, water availability, oxygen concentration, percent humidity, light availability, light composition, air composition, pot size, a greenhouse setting, a laboratory setting, a field setting, and/or temperature.

81. The method of claim 79, wherein said conditions are modified to a water-limited condition.

82. The method of claim 79, wherein said conditions are modified to limit an amount of soil.

83. The method of claim 79, wherein said seed comprises an open pollinated seed, a non-hybrid seed, a heirloom seed, a hybrid seed, a genetically modified organism (GMO) seed, or portions thereof.

84. The method of claim 79, wherein said exudates comprise sugars, fatty acids, amino acids, small peptides, polypeptides, organic acids, growth factors, enzymes, nucleotides, hormones, vitamins, alcohols, phenolics, volatiles, or stimulants.

85. The method of claim 79, wherein said sequencing is performed at least 7, 14, 21, or 30 days after said germinating.

86. The method of claim 85, wherein said sequencing is performed on a sample obtained from said rhizosphere.

87. The method of claim 79, further comprising performing bioinformatic analysis on results of said sequencing, optionally wherein said bioinformatic analysis comprises use of machine learning.

88. A method of characterizing one or more microorganisms of a sample, said method comprising:
a) contacting an attractant to said sample, wherein said attractant comprises a seed, an ovule, a nut, a kernel, a pit, a pip, a bulb, a grain, or portions thereof,
b) obtaining a rhizosphere of said sample; and
c) sequencing genetic material obtained from said rhizosphere, thereby characterizing said one or more microorganisms of said sample.

89. The method of claim 88, wherein said contacting comprises planting, incubating, soaking, submerging, burying, suspending, or encapsulating said attractant within said sample.

90. The method of claim 88, wherein said sequencing further comprises quantifying gene expression.

91. The method of claim 88, wherein said characterizing further comprises use of machine learning.

92. The method of claim 88, wherein said characterizing identifies rhizosphere health, soil health from which said sample is obtained, plant health, relative abundance of genes important to plant health, and/or a farming characteristic in a location wherein from which said sample is obtained.

93. A method for improving a plant characteristic, said method comprising:
a) obtaining a sample from a rhizosphere of said plant;
b) sequencing genetic material obtained from said sample;
c) performing bioinformatic analysis on results of said sequencing, optionally wherein said bioinformatic analysis comprises use of machine learning; and
d) determining a beneficial plant management technique for said plant to improve a plant characteristic based at least on said bioinformatic analysis.

94. The method of claim 93, wherein said beneficial plant management technique comprises identifying a beneficial seed type, growth temperature, water availability, humidity range, soil composition, soil treatment composition, nutrient composition, plant food, biofertilizer, biostimulant, seed type, or cultivar.

95. The method of claim 93, wherein said plant characteristic comprises one or more of: increased growth, increased disease resistance, increased resistance to an environmental stressor, increased pest resistance, increased pesticide resistance, increased nutrition efficiency, and/or improved crop quality.

96. The method of claim 95, wherein said environmental stressor comprises drought, flood, salinity, heat, cold, ozone, UV radiation, heavy metals, pollutants, or nutrient deficiency.

97. The method of claim 95, wherein said disease comprises stress, root rot, damping-off, vascular wilt, nutritional deficiency, salt injury, or infection caused by fungi, oomycetes, bacteria, viruses, viroids, virus-like organisms, phytoplasmas, protozoa, nematodes, or parasitic plants.

98. The method of claim 93, further comprising generating a score based on said bioinformatic analysis wherein said score takes into account one or more factors comprising one or more of: taxonomic profile, metagenomic profile, observed genera, community evenness, mycorrhizae, fungal/bacteria ratio (F/B ratio), rare biosphere, nitrification, denitrification, nitrogen fixation, phosphorous cycling, polymer degradation, drought resistance, disturbance, total score, or any combination thereof.
